(19) United States
(12) Patent Application Publication
(10) Pub. No.: US 2025/0361217 A1
(43) Pub. Date: Nov. 27, 2025

(54) FATTY ACID COMPOUNDS

(71) Applicants: University of Pittsburgh - Of the Commonwealth System of Higher Education, Pittsburgh, PA (US); The Regents of the University of California, Oakland, CA (US); Board of Supervisors of Louisiana State University and Agricultural and Mechanical College, Baton Rouge, LA (US)

(72) Inventors: Pascal Rowart, Pittsburgh, PA (US); Francisco J. Schopfer, Pittsburgh, PA (US); Sonia R. Salvatore, Pittsburgh, PA (US); Fei Chang, Pittsburgh, PA (US); Oren Rom, Bossier City, LA (US)

(21) Appl. No.: 18/873,917

(22) PCT Filed: Jun. 23, 2023
(86) PCT No.: PCT/US2023/068990

§ 371 Date: Dec. 11, 2024

Related U.S. Application Data
(60) Provisional application No. 63/355,290, filed on Jun. 24, 2022.

(51) Int. Cl. C07D 307/54; A61K 31/197; A61K 31/20; A61K 31/202; A61K 31/341; A61K 31/345; A61K 35/60; A61P 1/16; C07B 59/00; C07C 53/128; C07C 233/47; C07C 233/49; C07D 307/72

(52) U.S. Cl. CPC C07D 307/54; A61K 31/197; A61K 31/20; A61K 31/202; A61K 31/341; A61K 31/345; A61K 35/60; A61P 1/16; C07B 59/002; C07C 53/128; C07C 233/47; C07C 233/49; C07D 307/72

(57) ABSTRACT

Disclosed herein are furan fatty acid synthetic compounds and furan fatty acid—amino acid conjugates.

This application claims the benefit of U.S. Provisional App. 63/355,290, filed Jun. 24, 2022, which is incorporated herein by reference.

### ACKNOWLEDGMENT OF GOVERNMENT SUPPORT

This invention was made with government support under Grant Nos. AT009806; DK112854 and GM125944 awarded by the National Institutes of Health. The government has certain rights in the invention.

### BACKGROUND

Obesity is known to be a multifactorial disease that affects more than 35% of the world's population, reaching epidemic proportions worldwide. The prevalence of overweight humans in western countries brings an enormous risk of metabolic diseases as well as large healthcare costs. Obesity-related conditions include heart disease, stroke, non-alcoholic fatty liver diseases (NAFLD), non-alcoholic steatohepatitis, type 2 diabetes (T2D) mellitus, and certain types of cancer.

NAFLD is a chronic and increasingly common and well-defined liver disorder among adults. NAFLD is ultimately accompanied by different and distinct pathophysiological changes such as an increase in de novo lipogenesis (DNL) and production of very-low-density lipoprotein (VLDL), a decrease of hepatic fatty acids (FA) oxidation, and impaired insulin-mediated suppression of hepatic glucose production, leading to liver steatosis, hypertriglyceridemia, and hyperglycemia. All these factors are known to be clusters of metabolic syndrome (MetS).

Metabolic syndrome represents a cluster of metabolic defects related to insulin resistance, obesity, low-grade inflammation and dyslipidemia. Atherosclerotic cardiovascular disease (ASCVD) is the main cause of mortality for patients who have metabolic syndrome. Diabetic patients with dyslipidemia are exposed to a more significant ASCVD risk than diabetic patients without lipoprotein abnormalities. The polypharmacy approach (i.e., using specific treatments to normalize each dysregulated physiological risk factor) is generally adopted to treat this subset of diabetic patients. Specifically, the patients are primarily treated with low-density lipoprotein cholesterol (LDL-C) reducers (e.g., statins, ezetimibe, and/or PCSK9 inhibitors) for their dyslipidemia on top of already prescribed hypoglycemic medications. Clinical trials with diabetic patients (primarily Type2 Diabetes) have shown that the reduction of LDL-C positively correlated to a lower incidence of major adverse cardiac events. However, the benefits of statins are counterbalanced by a higher risk for myalgias and even can result in the occasional onset of diabetes in patients, leading to a lower maximum tolerated dosage or even discontinuation and low adherence to the treatment. More importantly, even if the patients tolerate statins for ASCVD treatment, a significant residual cardiovascular risk persists, especially in patients with diabetes.

Nonalcoholic fatty liver disease (NAFLD) covers a range of liver conditions affecting people who drink little to no alcohol. In subjects with NAFLD, too much fat is stored in liver cells. NAFLD is increasing in prevalence around the world, especially in Western nations, and is closely associated with obesity. In the United States, it is the most common form of chronic liver disease, affecting an estimated 80 to 100 million people. NAFLD is particularly prevalent in the age groups of 40-60 years in subjects that have other comorbidities including obesity and type 2 diabetes and are at high risk for heart disease. Patients with alcoholic liver disease share a similar set of health problems that arise from fat accumulation stemming from alcohol intake that leads to AFLD (alcoholic fatty liver disease).

NAFLD subjects may or may not exhibit physical symptoms of the disease. Symptoms include enlarged liver, fatigue, and pain in the upper right abdomen. Absent physical symptoms, NAFLD can be diagnosed through biochemical tests, such as liver enzymes and ultrasound elastography. NAFLD can be treated with bariatric surgeries including Roux-en-Y gastric bypass, sleeve gastrectomy or gastric banding, as they improve steatosis and steatohepatitis.

Nonalcoholic steatohepatitis (NASH) is a potentially serious form of the disease, which includes liver inflammation that can lead to scarring and irreversible damage. At its most severe, NASH can progress to cirrhosis and liver failure. NASH symptoms can include abdominal swelling, enlarged blood vessels just beneath the skin's surface, enlarged breasts in men, enlarged spleen, red palms and yellowing of the skin and eyes (jaundice). NASH is also strongly associated with obesity, dyslipidemia, type 2 diabetes, and metabolic syndrome. Currently, no NASH-specific therapies are approved by the US Food and Drug Administration.

Accumulating evidence has suggested favorable effects of fish oil (FO) on weight loss. FO-derived diets provide essential omega-3 (Ω-3) FA and have been shown to prevent liver steatosis, hypertriglyceridemia, FA synthesis, and reduce fat accumulation in human and animal models. FO and Ω-3 are effective treatments for hypertriglyceridemia, with eicosapentaenoic and docosahexaenoic acid as well as their respective ethyl esters having shown to reduce metabolic syndrome-related events. Lovaza™, a Ω-3-acid ethyl ester prescription, is currently used to reduce hypertriglyceridemia in humans. It has been reported that Lovaza™ treated patients showed a high level of a specific metabolite in urine and plasma which was identified as 3-carboxy-4-methyl-5-propyl-2-furanpropanoic acid (CMPF) (Prentice, K. J. et al. CMPF, a Metabolite Formed Upon Prescription Omega-3-Acid Ethyl Ester Supplementation, Prevents and Reverses Steatosis. EBioMedicine 27, 200-213 (2018).) In humans, CMPF is produced by the metabolism of Furan FAs (FuFAs) and therefore is considered an urofuranoic acid. FuFAs are lipids synthesized by algae and bacteria and found in plant- and fish-derived products. Nevertheless, it has been proven that fish, rats, and humans cannot synthesize FuFAs de novo.

FuFAs are known to exert anti-inflammatory (Chang, F, Hsu, et al., Synthesis of anti-inflammatory furan fatty acids from biomass-derived 5-(chloromethyl)furfural. Sustain. Chem. Pharm. 1, 14-18 (2015); Lee, E. S. et al. Potent analgesic and anti-inflammatory activities of 1-furan-2-yl-3-pyridin-2-yl-propenone with gastric ulcer sparing effect. Biol Pharm Bull 29, 361-364 (2006); Tenikoff, D. et al, (Lyprinol (stabilised lipid extract of New Zealand green-lipped mussel): a potential preventative treatment modality for inflammatory bowel disease J. Gastroenterol. 361-365 (2005) doi:10.1007/s00535-005-1551-x; Whitehouse, M. W. I. et al. Anti-inflammatory activity of a lipid fraction (Lyprinol) from the NZ green-lipped mussel. Inflammooharmacology 5, 237-246 (1997)); Wakimoto, T. et al. Furan fatty acid as an anti-inflammatory component from the green-lipped mussel Perna canaliculus Toshiyuki. Proc Natl Acad Sci USA 108, 17533-17537 (2011) doi: 10.1073/pnas.1110577108.), and anti-oxidant effects (Xu, L. et al. Furan fatty acids—Beneficial or harmful to health? Prog. Lipid Res. 68, 119-137 (2017); Okada, Y. et al.

Antioxidant Effect of Naturally Occurring Furan Fatty Acids on Oxidation of Linoleic Acid in Aqueous Dispersion Youji. J. Am. Oil Chem. Soc. 11, 858-862 (1990); Batna, A. & Spiteller, G. Effects of soybean lipoxygenase-1 on phosphatidylcholines containing furan fatty acids. Lipids 29, 397-403 (1994)).

Dysregulation of lipid metabolism has long been established as the culprit of NAFLD and metabolic syndrome. More recently, alteration in amino acid metabolism has emerged as a critical component of NASH progression, shared among other metabolic disorders, including cardiovascular disease (CVD). N-acyl amino acids (NAAs), composed of a fatty acid and an amino acid fused by an amide bond (e.g., N-oleoyl leucine or C18:1-Leu), integrate lipid and amino acid signaling and have lower levels in plasma in NASH patients. Supplementation via intraperitoneal administration of mice with C18:1-Leu restored the plasma levels and metabolic homeostasis, improved steatosis, and lowered inflammation and fibrosis in mice with established NASH (see PCT/US2021/46357). A significant upregulation of fatty acid β-oxidation (FAO) pathways through PPARα activation, along with downregulation of pro-inflammatory/fibrotic pathways (ccl2, NF-κB suppression) in the liver contributed to the improvement in NASH. Although the endogenous NAAs exhibited promising pharmacological benefits in metabolism and anti-inflammation, their oral bioavailability was low as enzymes in the gut readily degraded the compounds, preventing an oral administration.

### SUMMARY

A compound, or a pharmaceutically acceptable salt or ester thereof, having a structure of:

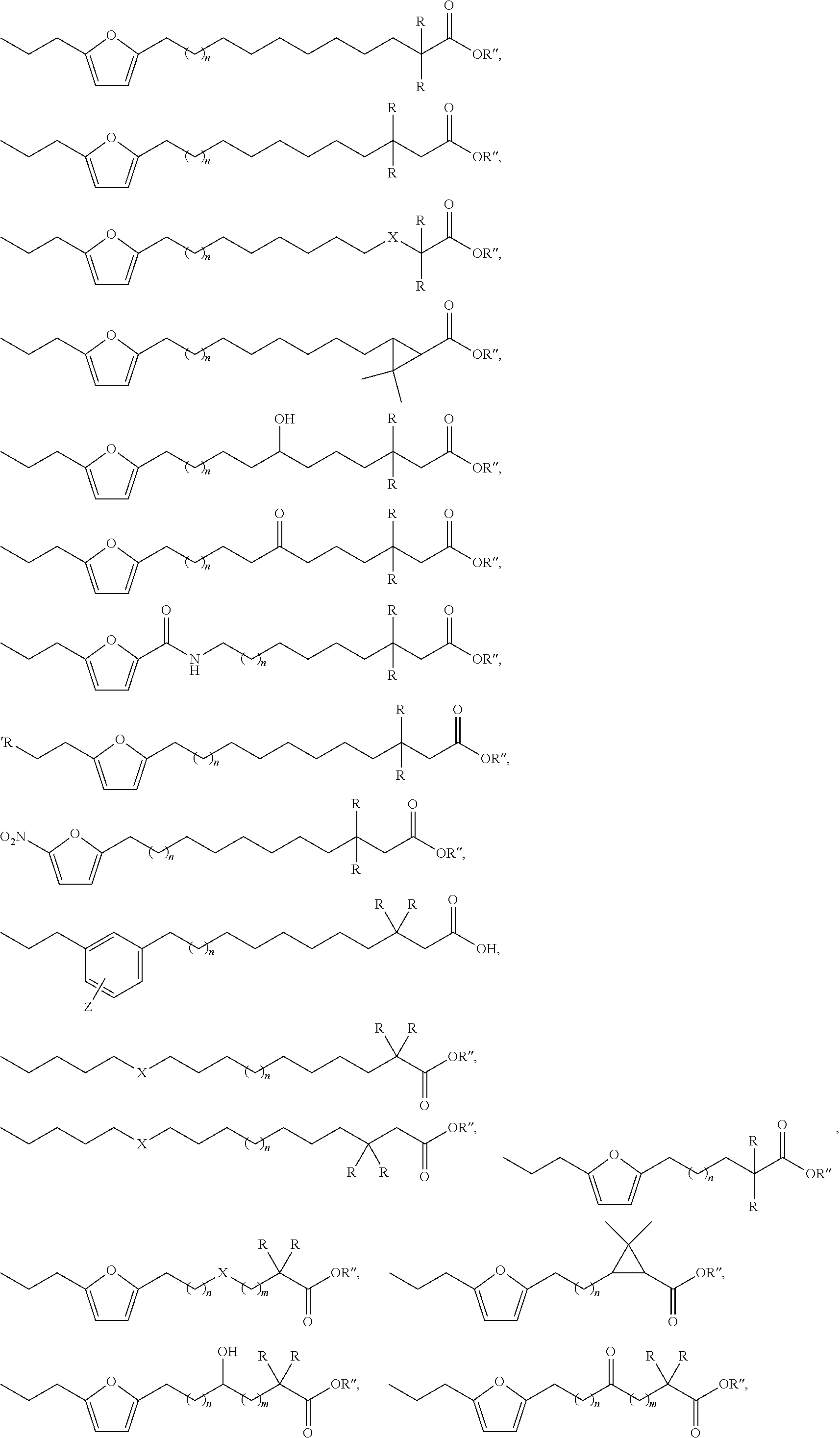

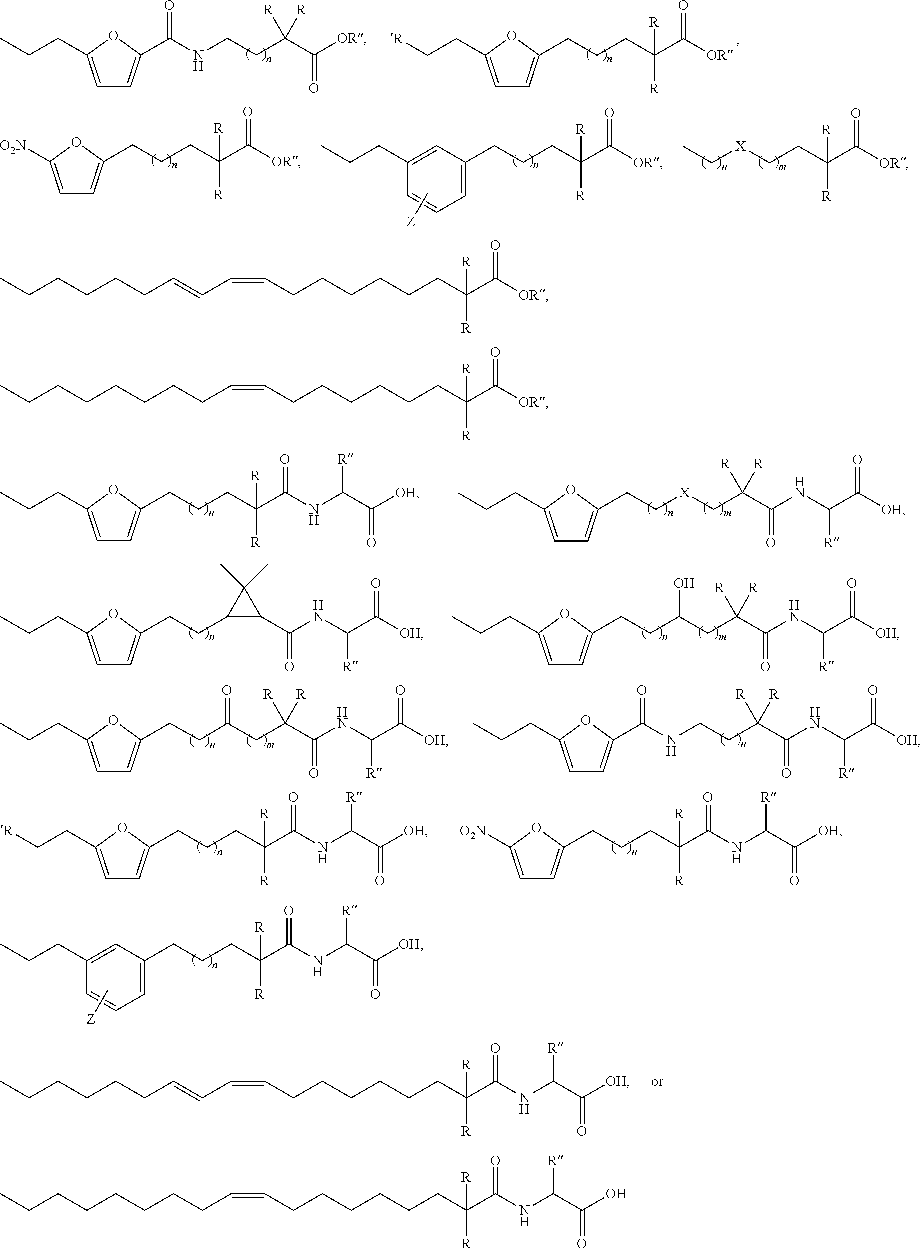

wherein each R is independently hydrogen, a C₁-C₆ alkyl, a C₁-C₆ haloalkyl, or a halogen, provided at least one R is a C₁-C₆ alkyl, a C₁-C₆ haloalkyl, or a halogen; X is O, S, or NH; ′R is a C₁-C₆ alkyl; Z is C₁-C₆ alkyl, C₁-C₆ haloalkyl, or halogen; R″ is hydrogen or alkyl; R′″ is a side chain of a natural L-amino acid; n is 1 to 10; and m is 0-10.

Also disclosed herein is a method comprising administering a therapeutically effective amount of any compound disclosed herein to a subject for treating metabolic syndrome, lipid abnormalities, insulin resistance, inflammation, heart disease, cardiovascular disease (e.g., atherosclerotic cardiovascular disease (ASCVD), stroke, non-alcoholic fatty liver diseases (NAFLD), steatosis (e.g, non-alcoholic steatohepatitis), type 2 diabetes (T2D) mellitus, dyslipidemia, hypertriglyceridemia, immune and inflammatory conditions that require a metabolic switch from oxidative phosphorylation to glycolysis in immune and inflammatory cells, skin-associated lipid diseases, acne, hidradenitis suppurativa, osteoarthritis, Crohn's disease, psoriasis, rheumatoid arthritis, or obesity-related, cancer in the subject. Other diseases include polycystic ovary syndrome (PCOS), a hormonal disorder affecting women characterized by enlarged ovaries containing small cysts that involves metabolic abnormalities such as insulin resistance, impaired glucose tolerance, and dyslipidemia, which contribute to the clinical manifestations of the syndrome. Wilson's disease, a rare genetic disorder with a mutation in the copper transport that affects primarily the liver and brain, leading to metabolic dysfunction and organ damage. Rheumatoid arthritis (RA) a chronic autoimmune disease characterized by joint inflammation where macrophages, fibroblast-like synoviocytes, and T cells undergo a metabolic switch with increased reliance on glycolysis for energy production, contributing to the inflammatory process. Psoriasis, a chronic inflammatory skin condition where immune cells, including T cells and dendritic cells, undergo a metabolic shift towards glycolysis, promoting the pro-inflammatory cytokine production and the pathogenesis of the disease. Inflammatory bowel disease, encompassing conditions such as Crohn's disease and ulcerative colitis, characterized by chronic inflammation of the gastrointestinal tract where immune cells present in the inflamed gut favor glycolysis to promote inflammation and tissue damage. Systemic lupus erythematosus, a systemic autoimmune disease where metabolic reprogramming of the immune system leads to an increased reliance on glycolysis, contributing to immune cell activation and tissue damage. Multiple sclerosis, a chronic inflammatory demyelinating disease of the central nervous system, with metabolic reprogramming towards glycolysis in activated immune cells, particularly T cells and microglia to support neuroinflammation and neurodegeneration. Similar switches towards glycolysis, that are prevented by our disclosed compounds, are mechanistically important and contribute to diseases including sepsis (macrophage and neutrophil), asthma (eosinophils and T cells), osteoarthritis (immune cells and chondrocytes), Chronic obstructive pulmonary disease (COPD) (neutrophils and macrophages), cancers rely on metabolic switches that support tumor growth, survival, and proliferation including breast cancer (epithelial cells), leukemia (blood cells), and glioblastoma (brain cells), non-small cell lung cancer, heart failure and myocardial infarction, with cardiomyocytes exhibiting metabolic alterations from fatty acid oxidation to glycolysis, Hashimoto's disease (lymphocytes and macrophages rewiring towards glycolysis), chronic kidney disease (proximal tubular cells), Parkinson's disease (dopaminergic neurons in the brain) experience metabolic changes, including a shift towards glycolysis. This metabolic switch is thought to contribute to neuronal dysfunction and neurodegeneration. Amyotrophic lateral sclerosis (ALS) and its motor neurons shift towards glycolysis and mitochondrial dysfunction, scleroderma (fibroblasts, immune cells, and endothelial cells metabolic changes), ischemic stroke (neurons and glial cells), osteosarcoma (malignant osteoblasts), glioblastoma (cancerous cell increased glycolysis), autoimmune hepatitis (lymphocytes and macrophages), colorectal cancer, primary biliary cholangitis, immune cells, such as lymphocytes, exhibit metabolic changes, including increased glycolysis.

Further disclosed herein is a method comprising administering a therapeutically effective amount of any compound disclosed herein to a subject for treating nonalcoholic steatohepatitis (NASH) in the subject.

Additionally disclosed herein is a method comprising administering a therapeutically effective amount of any compound disclosed herein to a subject for treating TG-induced pancreatitis and monogenetic dyslipidemia in the subject.

Also disclosed herein is a method comprising co-administering any compound disclosed herein and fish oil to a subject.

Also disclosed herein is a method comprising co-administering any compound disclosed herein and omega-3 to a subject.

Also disclosed herein is a method comprising co-administering any compound disclosed herein, fish oil and omega-3 to a subject.

The foregoing will become more apparent from the following detailed description, which proceeds with reference to the accompanying figures.

### BRIEF DESCRIPTION OF THE DRAWINGS

FIGS. 1A-1C: Detection and characterization of natural Furan fatty acids. (FIG. 1A) Chemical structure and nomenclature of most common natural FuFAs. (FIG. 1B) NEM-derivatization technique for detection and quantification by liquid chromatography-mass spectrometry (LC-MSMS) and various FuFA detected and present in Lovaza™ and fish oils. (FIG. 1C) Percentage of detectable FuFA and metabolites in fish oil, Lovaza™ and human plasma.

FIGS. 2A-2B: Chemical design of 11U3-2M increases its stability, prevents its oxidation, and reduces its incorporation in fatty acids. (FIG. 2A) Structures of natural and novel FuFA (FIG. 2B) Orally and intraperitoneally administered 11D3 an 11U3 are rapidly esterified and circulate systemically esterified to triglycerides. In contrast, 11U3-2M is not esterified and circulates as free acid.

FIGS. 3A-3D: 11U3-2M is protective in a diet-induced murine model of NAFLD, reverses steatosis and improves glucose clearance and insulin. (FIG. 3A) Treatment paradigm (FIG. 3B) 11U3-2M significantly improves glucose handling in a OGTT challenge (FIG. 3C). 11U3-2M significantly reduces insulin, C-peptide 2, and GIP circulating levels, indicative of improved insulin sensitivity (FIG. 3D). 111U3-2M significantly improves liver morphology and steatosis, improving liver enzymes (AST and ALT) and reducing inflammation (TNFα).

FIGS. 4A-4C: 11U3-2M decreases plasma levels of cholesterol-ester and triglycerides and increases metabolic changes with a major effect on sphingolipids and acylcarnitine in livers from animals in a diet-induced murine model of NAFLD. (FIG. 4A) 11U3-2M significantly reduces circulating cholesterol esters and triglycerides. (FIGS. 4B-4C) 11U3-2M normalizes acylcarnitine conjugates and ceramides dysregulated by the high-fat diet.

FIGS. 5A-5E: 11U3-2M activates pathways involved in fatty acids D-oxidation, mitochondria and PPARa in a mouse model of NAFLD. (FIGS. 5A-5B) 11U3-2M induces dramatic liver reprogramming with a significant increase in fatty acid oxidation pathways as assessed by RNAseq. (FIGS. 5C-5E) Dramatic increases in long non-coding RNA GM 15441 were induced by the treatment of 11U3-2M. RNAseq data pathway confirmation using PCR and fluorescence in-situ hybridization.

FIGS. 6A-6D: 11U3-2M induces a metabolic switch in the liver to decrease glycolysis and increase mitochondrial function and oxidative phosphorylation. increasing the number of mitochondria and complex activity. (FIG. 6A) Treatment scheme. (FIG. 6B) Citrate synthase activity (CSA) is increased in mice treated with 11U3-2M reflective of an increase in mitochondrial mass. (FIG. 6C) Increased activity of electron flux in liver mitochondria after treatment with 11U3-2M. (FIG. 6D) Animals show no change in energy expenditure or respiratory exchange ratio after treatment with 11U3-2M.

FIGS. 7A-7D: Formation of 11U3-2M-CoA adduct and its effect on fatty acids β-oxidation by direct inhibition of ACC and ACLY. (FIG. 7A) 11U3-2M results in significant conjugation to Coenzyme A (CoA) and stability of the conjugate in liver (FIG. 7A) and primary hepatocytes (FIG. 7B). (FIG. 7C-7D) Increased 11U3-2M-CoA adducts in the liver from treated animals increase the thermal stability of ACC and ACLY, key metabolic enzymes that regulate fatty acid metabolism and de novo lipogenesis.

FIG. 8: Synthetic route for 11U3-2M

FIGS. 9-17: Spectra of compounds disclosed herein.

FIGS. 18A-18D: Cassette PK study of branched vs. straight-chain N-acyl amino acid (NAA) conjugates, exemplified by 11U3-2M-Leu, 11U3-Leu, and C18:1-Leu (30 mg/kg each). Greatly improved oral bioavailability due to the dimethylation was observed. (FIG. 18A) Plasma levels of FA-AA (NAA) conjugates upon oral administration. (FIG. 18B) Liver levels of FA-AA conjugates upon oral administration. (FIG. 18C) Plasma levels of FA-AA conjugates upon IP injection. (FIG. 18D) The concentrations of 11U3-2M and 11U3 in the liver upon oral and IP administration of their corresponding Leun conjugates, demonstrating a slow release of the secondary bioactive fatty acids.

FIG. 19: Reduced liver damage by 11U3-2M-Leu was confirmed by gross morphology. Gross appearance of peritoneal cavities and livers were shown. The hepatomegaly induced by NASH diet was also attenuated by 11U3-2M-Leu treatment resulting in a decrease in liver weight and liver-to-body weight ratio. Data are mean±SEM. Data were tested for normality using Shapiro-Wilk test followed by one-way ANOVA and Tukey post-hoc test. All individual points and p values are shown (n=8-12). A p-value of less than 0.05 is considered significant. *p<0.05, **p<0.01.

FIG. 20: Animals treated with I1U3-2M-Leu demonstrated significantly reduced circulating AST and ALT along with total cholesterol levels. Data are mean±SEM. Data were tested for normality using Shapiro-Wilk test followed by one-way ANOVA and Tukey post-hoc test. All individual points and p values are shown. All individual points and p-values are shown (n=8-12). A p-value of less than 0.05 is considered significant. *p<0.05, **p<0.01, ***p<0.001, ****p<0.0001.

FIG. 21: Genes related to fatty acid oxidation were upregulated with the 11U3-2M-Leu treatment. Data are mean±SEM. Data were tested for normality using Shapiro-Wilk test followed by one-way ANOVA and Tukey post-hoc test. All individual points and p-values are shown. A p-value of less than 0.05 is considered significant. *p<0.05, **p<0.01, ***p<0.001, ****p<0.0001.

FIG. 22: Concurrent suppression of lipogenesis by the treatment of 11U3-2M-Leu was discovered based on the expression of the genes related to de novo lipogenesis. Data are mean±SEM. Data were tested for normality using Shapiro-Wilk test followed by one-way ANOVA and Tukey post-hoc test. All individual points and p-values are shown. A p-value of less than 0.05 is considered significant. *p<0.05, **p<0.01, ***p<0.001, ****p<0.0001.

FIG. 23: 11U3-2M-Lcu treatment showed both anti-inflammatory and anti-fibrotic effects in the NASH animals. Data are mean±SEM. Data were tested for normality using Shapiro-Wilk test followed by one-way ANOVA and Tukey post-hoc test. All individual points and p-values are shown. A p-value of less than 0.05 is considered significant. *p<0.05, **p<0.01, ***p<0.001, ****p<0.0001.

FIG. 24: Spectra of a compound disclosed herein.

FIGS. 25A-25B: Plasma levels after intraperitoneal administration.

FIGS. 26A-26B: Plasma levels after oral administration.

FIGS. 27A-27B: Liver tissue levels after intraperitoneal administration.

FIGS. 28A-28B: Liver tissue levels after intraperitoneal administration.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, bulanoyloxy, isobutanoyloxy, pentanoyloxy, or hexanoyloxy.

An “analog” is a molecule that differs in chemical structure from a parent compound, for example a homolog (differing by an increment in the chemical structure or mass, such as a difference in the length of an alkyl chain or the inclusion of one of more isotopes), a molecular fragment, a structure that differs by one or more functional groups, or a change in ionization. An analog is not necessarily synthesized from the parent compound. A derivative is a molecule derived from the base structure.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals. Illustrative non-human mammals include animal models (such as mice), non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

The term “co-administration” or “co-administering” refers to administration of a compound disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

Cyclic: Designates a substantially hydrocarbon, closed-ring compound, or a radical thereof. Cyclic compounds or substituents also can include one or more sites of unsaturation, but does not include aromatic compounds. One example of such a cyclic compound is cyclopentadienone.

The term “cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, and the like. The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “ester” refers to a carboxyl group-containing moiety having the hydrogen replaced with, for example, a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OCOMe, —OCOEt and —OCOPr).

“Halo” or “halogen”, as used herein, refers to fluoro, chloro, bromo, and iodo.

The terms “halogenated alkyl” or “haloalkyl group” refer to an alkyl group with one or more hydrogen atoms present on these groups substituted with a halogen (F, Cl, Br, I).

The term “heterocyclic” refers to a closed-ring compound, or radical thereof as a substituent bonded to another group, particularly other organic groups, where at least one atom in the ring structure is other than carbon, and typically is oxygen, sulfur and/or nitrogen.

The term N-acyl amino acid (NAA) refers to a class of compounds in which an acyl group is attached to an amino acid molecule. In these compounds, the acyl group is linked to the amino group (—NH2) of the amino acid, forming an amide bond.

“Inhibiting” refers to inhibiting the full development of a disease or condition. “Inhibiting” also refers to any quantitative or qualitative reduction in biological activity or binding, relative to a control.

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆ alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl, particularly N-methyl.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect on the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease. A “prophylactic” treatment is a treatment administered to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolyzed in the human or animal body to produce the parent acid or alcohol. Representative esters thus include amino acid esters (for example, L-glycil, L-leucyl or L-isoleucyl or any of the other natural aminoacids), carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl);. A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism, conjugation and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized or conjugated into the active inhibitors in vivo. Prodrugs of a compounds described herein may be prepared by modifying functional groups present in the compound in such a way that the modifications are cleaved, either by routine manipulation or in vivo, to the parent compound, these modifications are used to form the active compounds inside tissues and cells by conjugation to cellular organic molecules (e.g., Coenzyme A, carnitine and glycerol to provide an example). The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs may include compounds having a phosphonate, hydroxy, thio and/or amino group functionalized with any group that is cleaved in vivo to yield the corresponding amino, hydroxy, thio and/or phosphonate group, respectively. Examples of prodrugs can include, without limitation, compounds having an acylated amino group and/or a phosphonate ester or phosphonate amide group.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Particular examples of the presently disclosed compounds may include one or more asymmetric centers; thus the compounds described can exist in different stereoisomeric forms. Accordingly, compounds and compositions may be provided as individual pure enantiomers or as stereoisomeric mixtures, including racemic mixtures. In certain embodiments the compounds disclosed herein may be synthesized in or may be purified to be in substantially enantiopure form, such as in a 90% enantiomeric excess, a 95% enantiomeric excess, a 97% enantiomeric excess or even in greater than a 99% enantiomeric excess, such as in enantiopure form.

The presently disclosed compounds can have at least one asymmetric center or geometric center, cis-trans center(C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁵N, ¹³C, etc.

### Compounds

Disclosed herein are non-natural synthetic fatty acid and their N-amino acid conjugates. In certain embodiments, the compounds have improved physical/chemical properties than the natural products. In certain embodiments, the compounds have improved ADME profiles. In certain embodiments, the compounds have improved biological activity and pharmacological actions.

In certain embodiments, certain moieties (e.g., halogen (e.g., fluoro) group(s), cyclic aliphatic group(s), heteroatom(s), hydroxy(s), oxo group(s), amide group(s), alkyl group(s)) are inserted in the fatty acid chain, or along the fatty acid chain to inhibit beta-oxidation of the compounds, enabling a longer half-life. The increased permeability of these compounds and the absorption through the portal vein, as opposed to the lymphatic mechanism of natural fatty acids, also enable a higher liver deposition, rendering a stronger organ-specific efficacy. In certain embodiments, the methyl groups on the 3,4-positions of the furan ring in the natural product are replaced with moieties (e.g., hydrogen(s), fluorine(s), nitro, nitroso, halogenated alkyl(s), alkyl(s)) so that they will not afford carboxylic functionalities after metabolism, and thus prevent the formation of CMPF-like metabolites which display pancreatic toxicities. In certain embodiments, heteroatoms (e.g., O, N, or S) are inserted into the carboxyalkyl chain to interrupt the beta-oxidation of the compound.

In certain embodiments, the compound, or a pharmaceutically acceptable salt or ester, including amino acid ester, thereof, has a structure of:

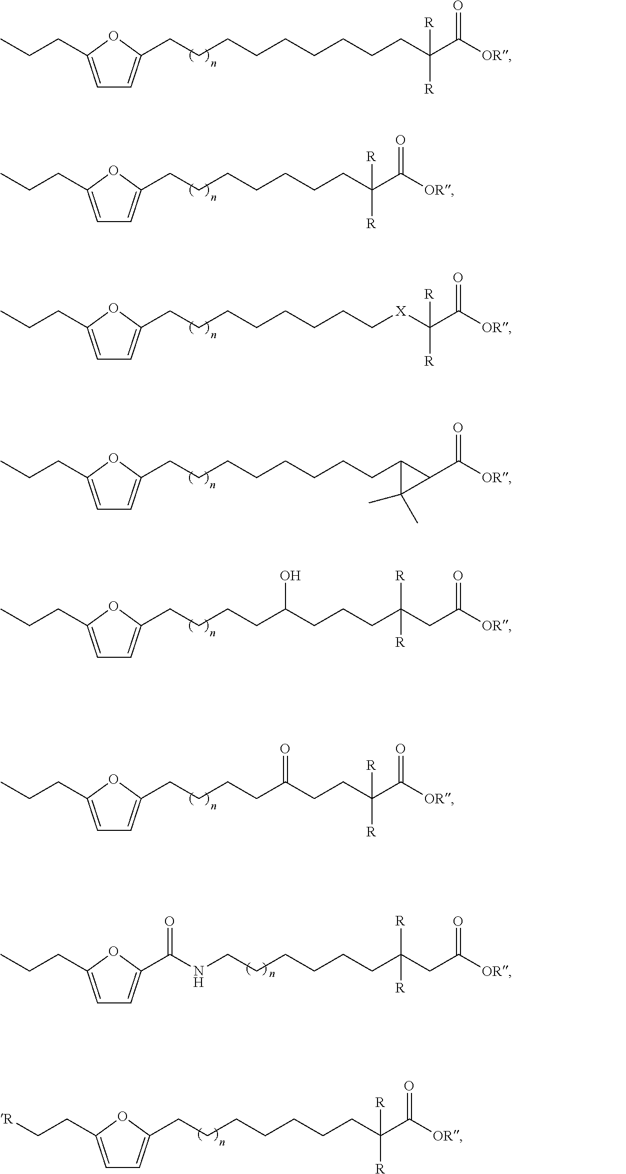

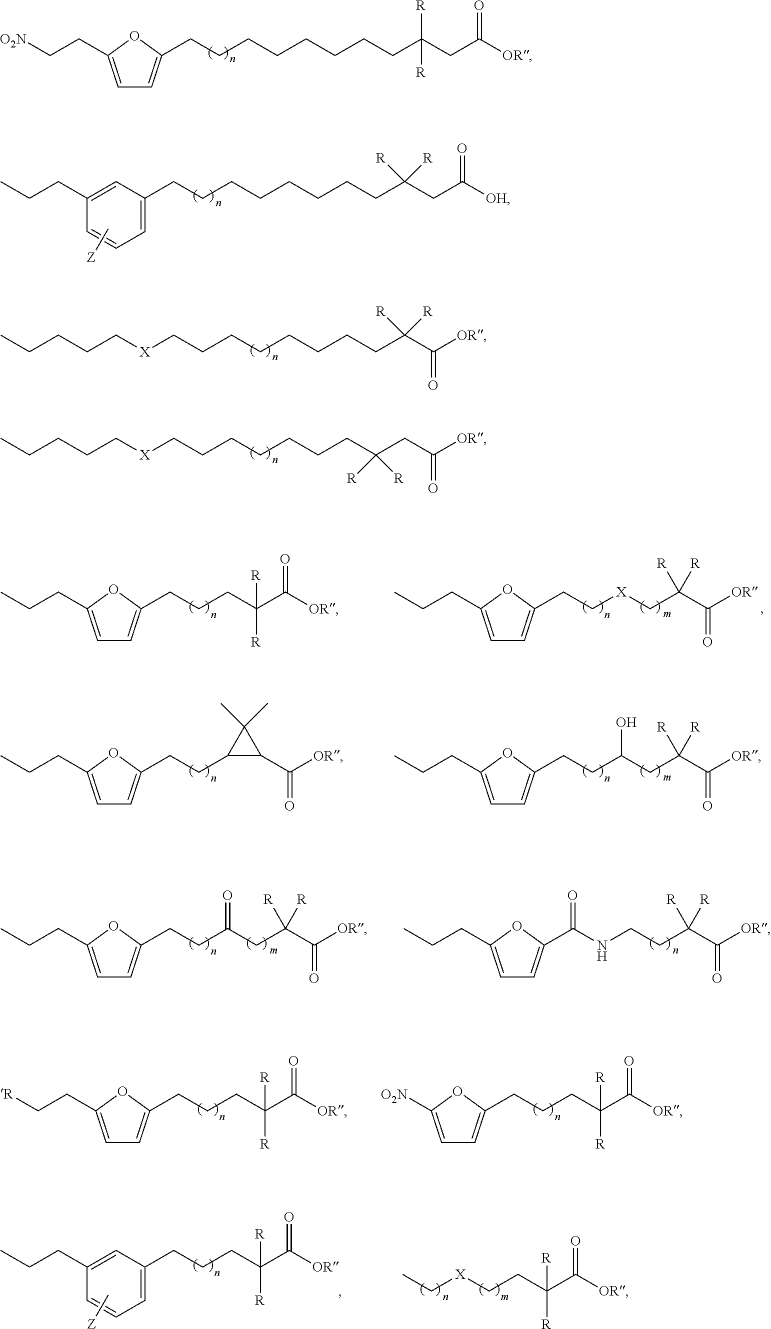

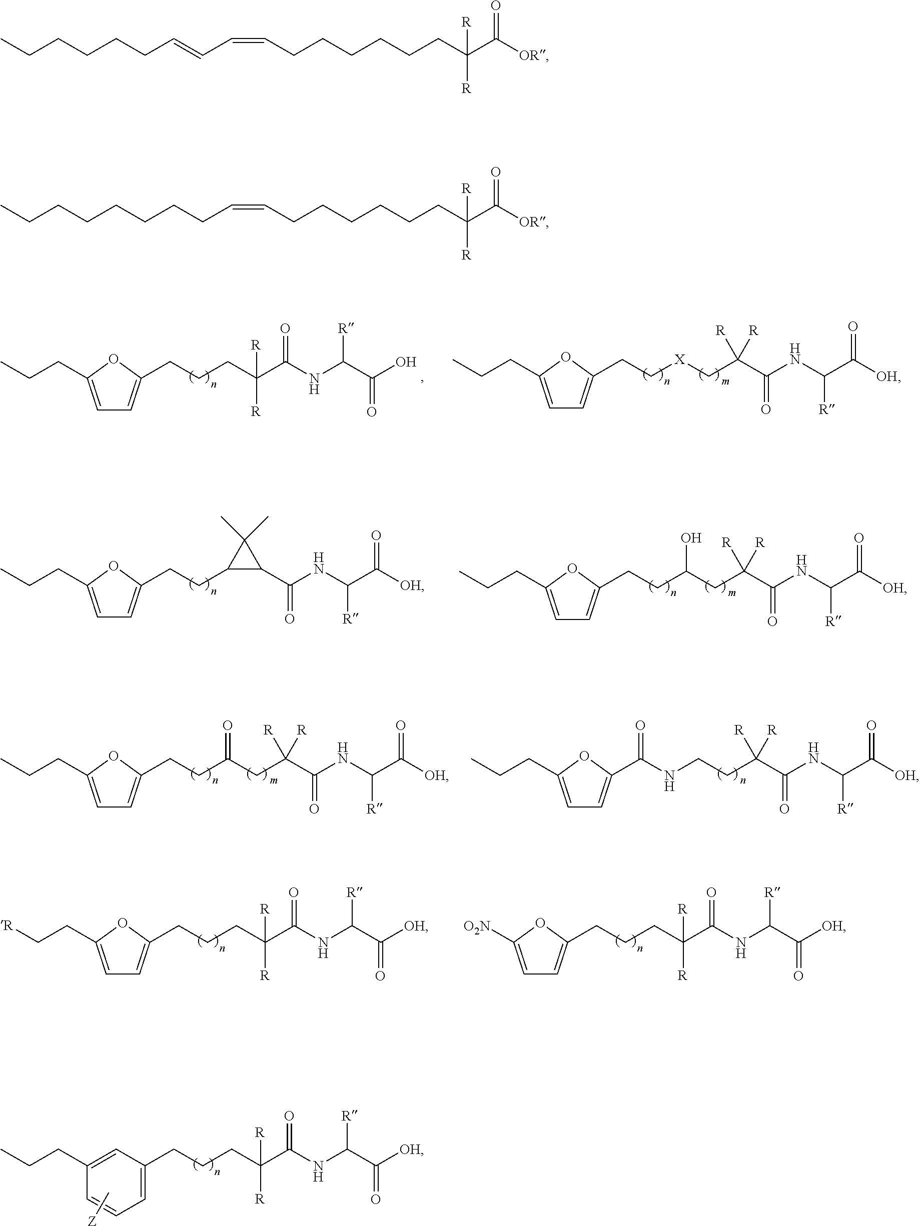

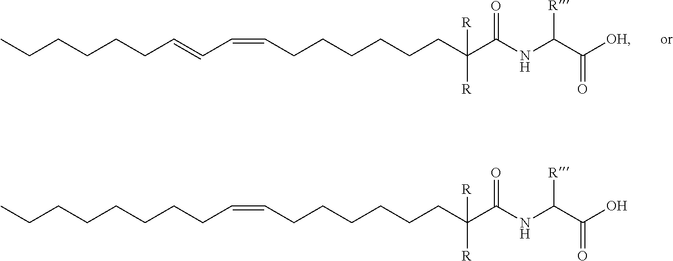

wherein each R is independently hydrogen, a C₁-C₆ alkyl, a C₁-C₆ haloalkyl, or a halogen, provided at least one R is a C₁-C₆ alkyl, a C₁-C₆ haloalkyl, or a halogen; X is O, S, or NH; ′R is a C₁-C₆ alkyl; Z is C₁-C₆ alkyl, C₁-C₆ haloalkyl, or halogen; R″ is hydrogen or alkyl; R′″ is a side chain of a natural amino acid; n is 1 to 10; and m is 0 to 10.

In certain embodiments, both R groups are not hydrogen.

In certain embodiments, both R groups are the same.

In certain embodiments, the R group is methyl, ethyl, propyl, isopropyl, flourine or chlorine.

In certain embodiments, n is 1.

In certain embodiments, R′″ is hydrogen or isobutyl group, representing glycine and leucine respectively.

Other illustrative compounds include fatty acid-amino conjugates such as:

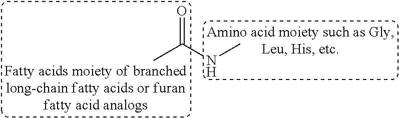

Additional illustrative compounds include:

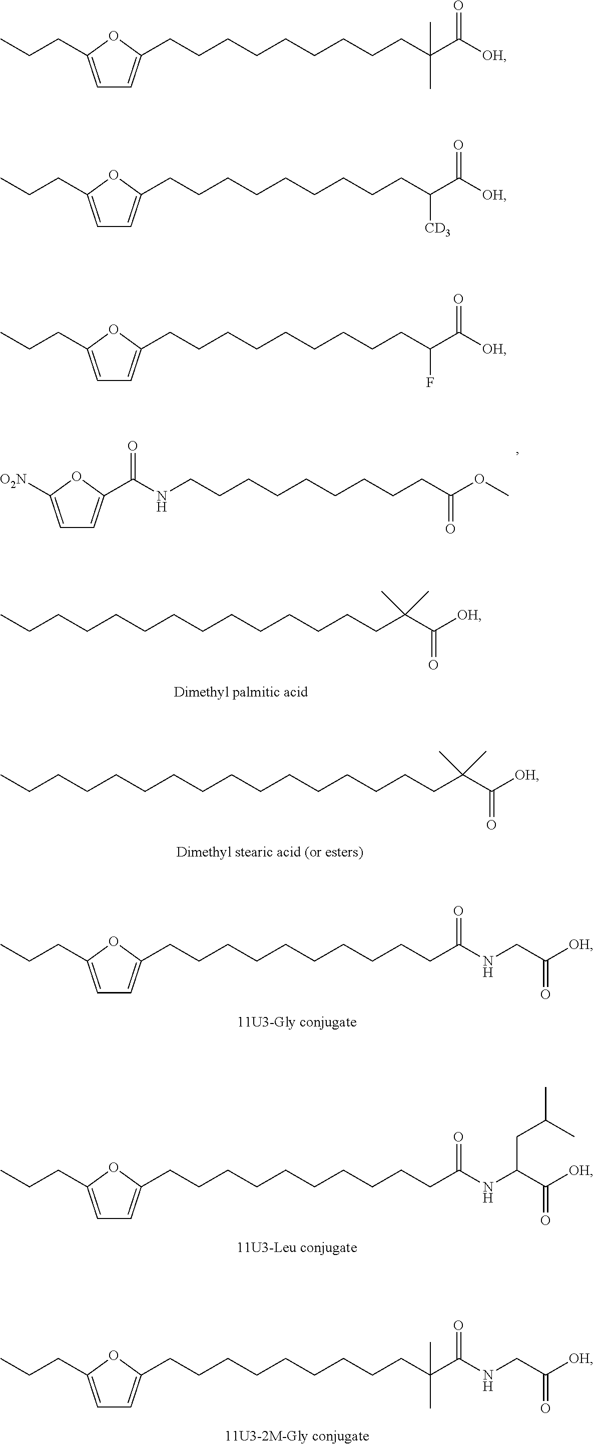

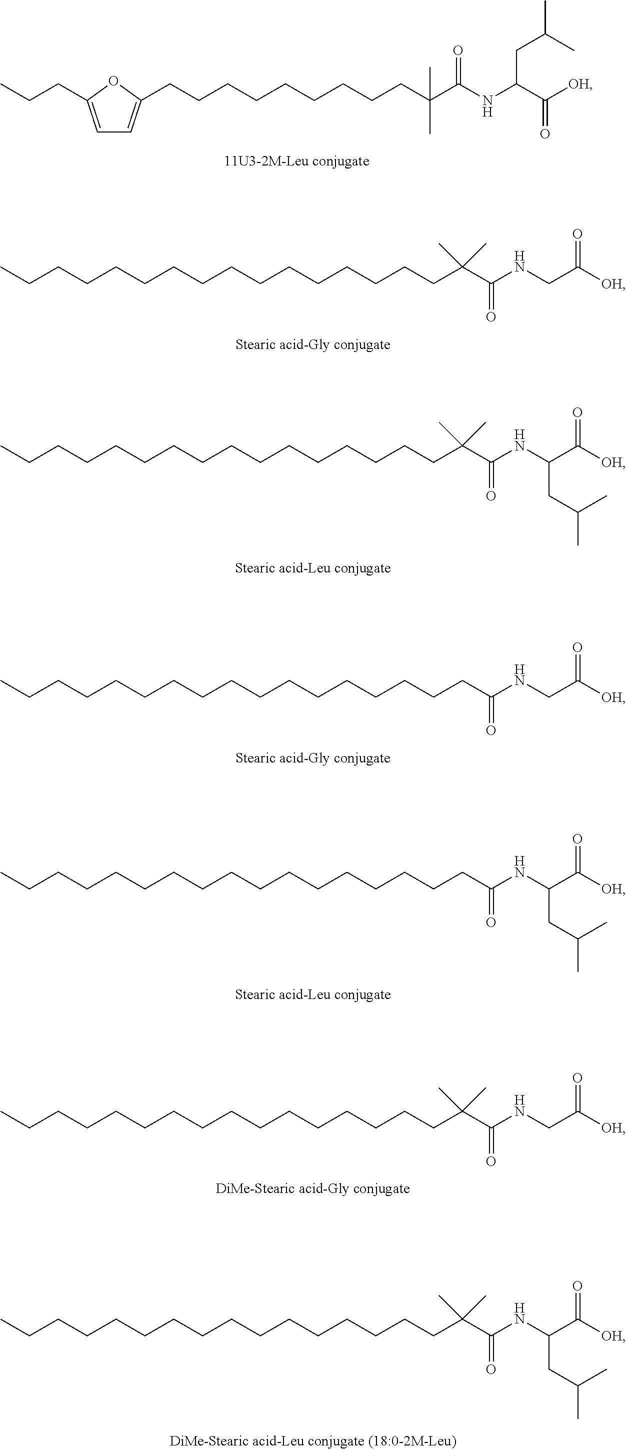

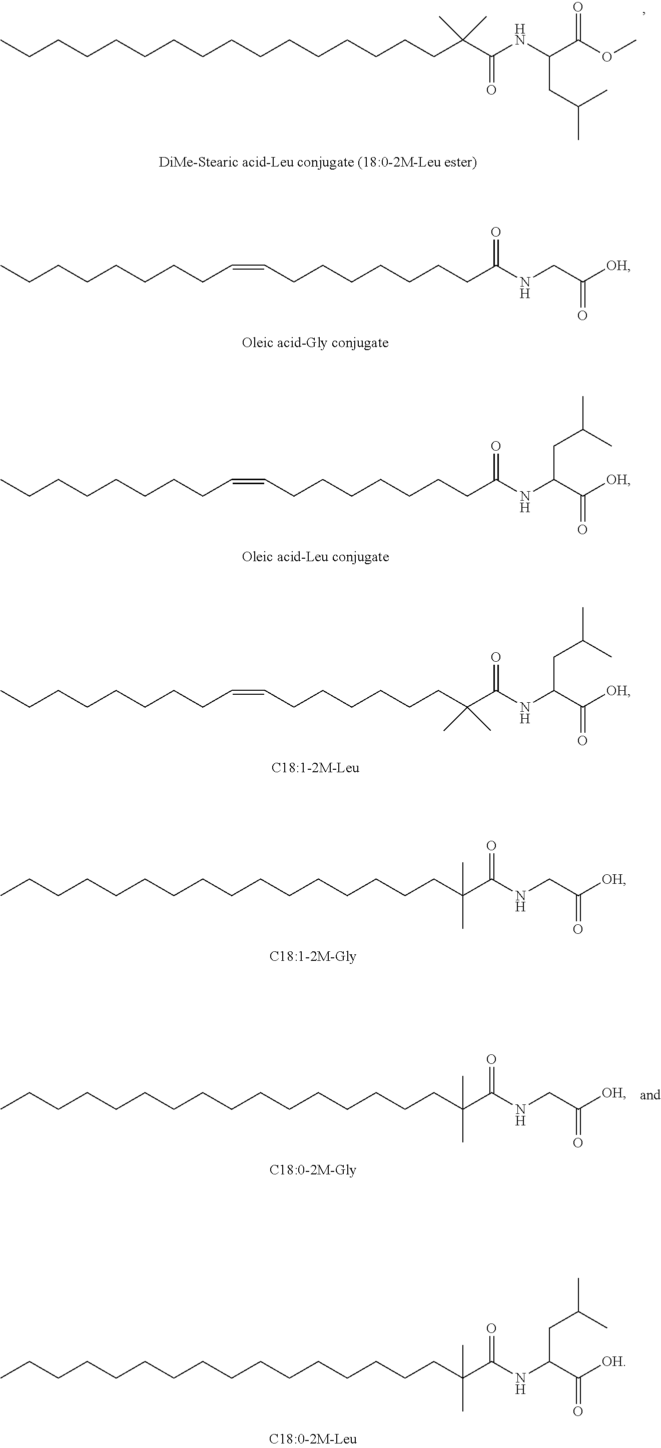

Illustrative synthesis procedures are shown below:

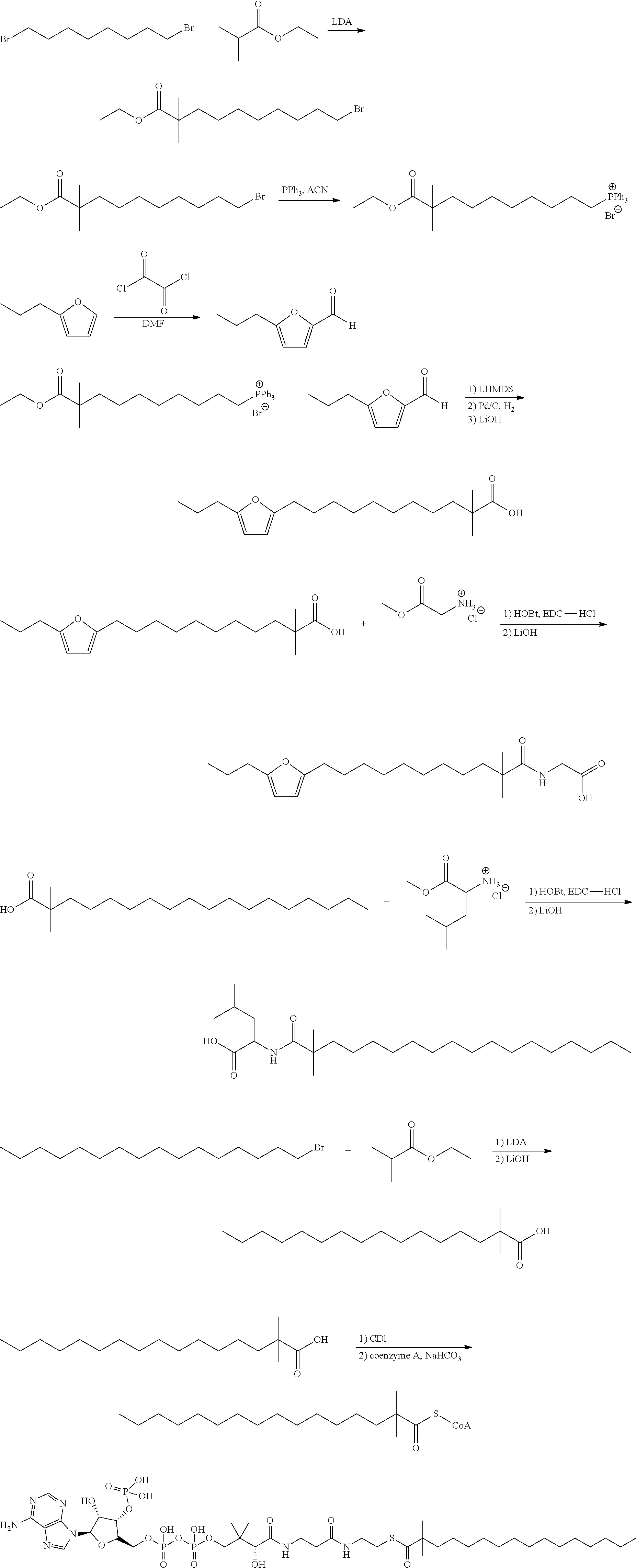

### Pharmaceutical Compositions and Methods of Treatment

In certain embodiments, the compounds disclosed herein can be used for treating metabolic syndrome. The compounds disclosed herein offer a novel treatment option using holistic metabolic reprogramming for subjects. For example, the compounds disclosed herein induce a metabolic reprogramming from glycolysis to fatty acid oxidation.

In certain embodiments, the compounds disclosed herein can be used for treating lipid abnormalities, insulin resistance, inflammation, heart disease, cardiovascular disease (e.g., atherosclerotic cardiovascular disease (ASCVD), stroke, non-alcoholic fatty liver diseases (NAFLD), steatosis (e.g, non-alcoholic steatohepatitis), type 2 diabetes (T2D) mellitus, dyslipidemia, hypertriglyceridemia, and certain types of obesity-related cancer.

In certain embodiments, the compounds disclosed herein can be used for treating a non-alcoholic fatty liver disease (NAFLD) in a subject.

In certain embodiments, the compounds disclosed herein can be used for treating fatty liver disease in a subject.

In certain embodiments, the compounds disclosed herein can be used for treating nonalcoholic steatohepatitis (NASH) in a subject.

In certain embodiments, the compounds disclosed herein can be used for treating cardiovascular disease, particularly cardiovascular diseases with other concurrent underlying metabolic dysfunctions (e.g., diabetes, obesity and metabolic syndrome).

In certain embodiments, the compounds disclosed herein can be used for treating including ischemic stroke heart failure, and myocardial infarction.

In certain embodiments, the compounds disclosed herein can be used for treating TG-induced pancreatitis and monogenetic dyslipidemia such as familial chylomicronemia.

In certain embodiments, the compounds disclosed herein can be used for treating immune or immunometabolic disorders.

In certain embodiments, the compounds disclosed herein are used to treat primary biliary cholangitis.

In certain embodiments, the compounds reduce atherosclerosis by simultaneously managing multiple metabolic risk factors (i.e., elevated TG and cholesterol, insulin resistance, and inflammation) of cardiovascular disease.

In certain embodiments, the compounds disclosed herein activate fatty acid oxidation, reduce circulating TG and cholesterol, normalize fasting glucose level, and/or reduce plasma pro-inflammatory biomarkers (e.g., TNFα).

In certain embodiments, the compounds disclosed herein to treat immune and inflammatory conditions that are characterized by a metabolic switch from oxidative phosphorylation to glycolysis.

In certain embodiments, the compounds disclosed herein are used to treat inflammatory and immune skin diseases, particularly acne, hidradenitis suppurativa, psoriasis, osteoarthritis.

In certain embodiments, the compounds disclosed herein are used to treat inflammatory and autoimmune diseases including osteoarthritis, Crohn's disease, ulcerative colitis rheumatoid arthritis, systemic lupus erythematosus, asthma, chronic obstructive pulmonary disease and chronic kidney disease.

In certain embodiments, the compounds disclosed herein are used to treat inflammatory conditions with underlying metabolic abnormalities including polycystic ovary syndrome (PCOS), Wilson's disease.

In certain embodiments, the compounds disclosed herein are used to treat cancer including leukemia, glioblastoma, breast cancer, pancreatic cancer, non-small cell lung cancer, lymphoma, liver cancer, lung cancer, colorectal cancer, melanoma, kidney cancer, osteosarcoma and glioblastoma.

In certain embodiments, the compounds disclosed herein are used to treat neurological disorders including multiple sclerosis, Parkinson's disease, and amyotrophic lateral sclerosis.

In certain embodiments, the compounds disclosed herein can be an alternative treatment to current omega-3 FDA approved hypolipidemic treatments.

In certain embodiments, the compounds inhibit acetyl CoA carboxylase exerting a metabolic shift responsible for the anti-steatotic effect.

In certain embodiments, the compounds disclosed herein exert anti-inflammatory effects through acetyl-CoA carboxylase inhibition.

In certain embodiments, the compounds disclosed herein exert anti-inflammatory effects through AMPK activation.

In certain compounds, the compounds disclosed herein exert anti-inflammatory effects through ATP Citrate Lyase inhibition.

In certain embodiments, the compounds regulate FGF21 function and levels.

In certain embodiments, the compounds are used to reprogram metabolism in cancer to kill the tumor cells.

In certain embodiments, the compounds are anti-inflammatory and modulate immune cell responses, cell activation, and cytokine production.

In certain embodiments, the subject is in need of, or has been recognized as being in need of, treatment for elevated TG, cholesterol, and/or insulin resistance.

In certain embodiments, the CoA conjugates can inhibit the enzyme lactate dehydrogenase (LDH).

Glycolytic metabolism is critical to support cancer cell growth and LDH plays an important role in this pathway. Inhibition of LDH is an important target for developing drugs to treat cancer therapeutics.

The compounds disclosed herein can be co-administered with fish oil and/or omega-3 to potentiate their effect.

In certain embodiments, the compounds can be co-administered with anti-obesity drugs (dual glucose-dependent insulinotropic polypeptide (GIP) and GLP-1 receptor agonist [e.g., tirzepatide and), anti-diabetic drugs (GLP-1 agonists [e.g., semaglutide], SGLT-2 inhibitors, metformins, thiazolidinediones, DPP-4 inhibitors, etc.); cholesterol-lowering drugs (e.g. statins, PCSK-9 inhibitors, ezetimibe); fibrates; niacin; or anti-hypertensive drugs (e.g. ace inhibitors, angiotensin 2 receptor inhibitors, etc.).

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compound can be formulated as cyclodextrin inclusion complexes.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single-dosage administration. The concentration of active compound in the drug composition will depend on absorption, inactivation, and excretion rates of the active compound, the dosage schedule, and amount administered as well as other factors known to those of skill in the art.

In some examples, about 1 mg to 4000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 5 mg to about 4000 mg (for example, about 1000 mg to about 4000 mg, about 100 mg to 1000 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 2000 mg, about 3000 mg, about 4000 mg or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 100 mg/kg body weight in single or divided doses. One illustrative dosage range is 0.1 to 100 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono-, di, or tri-glycerides, and fatty acids, including oleic acid, and phospholipids. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### Examples

### Compound Synthesis

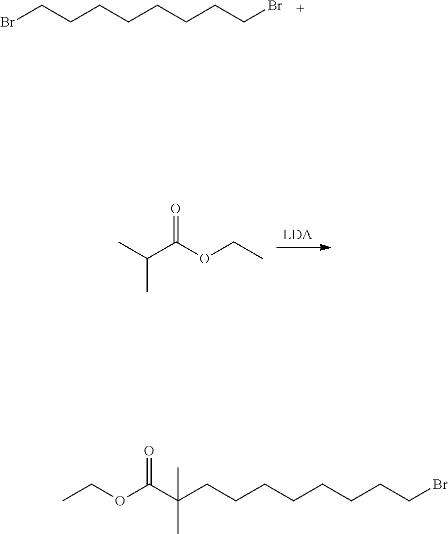

Ethyl10-bromo-2,2-dimethyldecanoate

To a solution of 1,8-dibromooctane (2.80 g, 10.2 mmol) and ethyl isobutyrate (1.00 g, 8.62 mmol) in THF (20 mL) was added dropwise lithium diisopropylamide (2M in THF, 5.1 mL) at −78° C. under nitrogen. The mixture was gradually warmed up to rt and stirred overnight before quenched with an ice-cooled NH₄Cl aq. solution. The mixture was extracted with ethyl acetate three times. The combined organic layers were dried over NaSO₄ and evaporated under vacuum. The crude product was purified by SiO₂ column (0-20% ethyl acetate in hexanes) to give the desired ethyl 10-bromo-2,2-dimethyldecanoate (1.541 g, 58%).

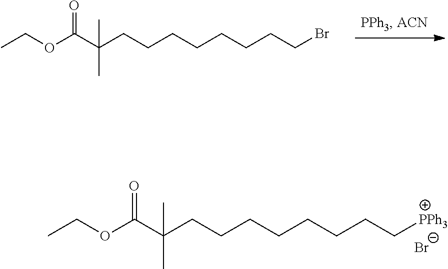

(10-Ethoxy-9,9-dimethyl-10-oxodecyl)triphenylphosphonium bromide

To a solution of ethyl 10-bromo-2,2-dimethyldecanoate (575 mg, 1.87 mmol) in acetonitrile was added triphenylphosphine (542 mg, 2.07 mmol). The mixture was heated up to 70° C. and stirred for 3 days. The reaction mixture was then concentrated under vacuum and purified by SiO₂ column (0-15% MeOH in dichloromethane) to give the desired (10-ethoxy-9,9-dimethyl-10-oxodecyl)triphenylphosphonium bromide as light yellow oil (836 mg, 78%).

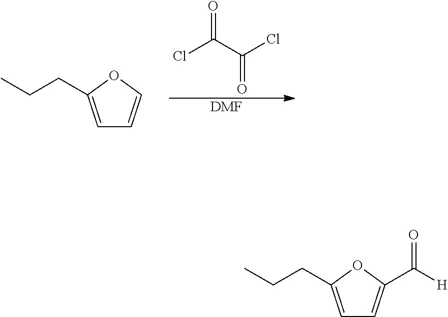

5-Propylfuran-2-carbaldehyde

Oxalyl chloride (0.43 mL, 5.0 mmol) was added dropwise to N,N-dimethylformamide (0.385 mL, 5.00 mmol) at 10° C. The solution was stirred at rt for 15 min. To this solution was added dichloromethane (10 mL) followed by 2-propylfuran (500 mg, 4.55 mmol) in dichloromethane (5 mL) over 5 min. The resulting mixture was stirred at rt for 30 min before sodium acetate (2 g) in water (20 mL) was added. The mixture was stirred for an additional 30 min. The mixture was extracted with dichloromethane three times. The combined organic layers were dried over NaSO₄ and evaporated under vacuum. The crude product was purified by SiO₂ column (0-25% ethyl acetate in hexanes) to give the desired 5-propylfuran-2-carbaldehyde (505 mg, 81%).

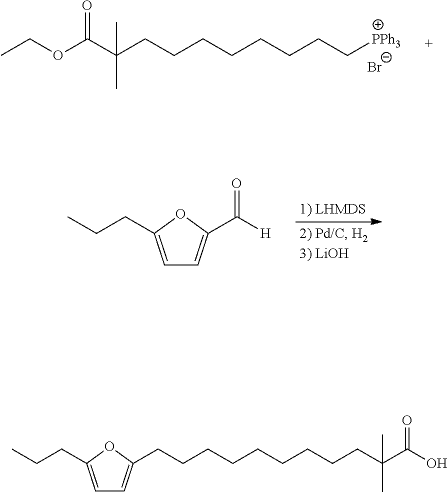

2,2-Dimethyl-11-(5-propylfuran-2-yl)undecanoic acid

To a solution of (10-ethoxy-9,9-dimethyl-10-oxodecyl)triphenylphosphonium bromide (3.547 g, 6.234 mmol) in THF (10 mL) and DMSO (10 mL) was added lithium bis(trimethylsilyl)amide (1M in THF, 6.5 mL) at 0° C. The mixture was stirred at the same temperature for 30 min before 5-propylfuran-2-carbaldehyde (860 mg, 6.23 mmol) in THF (10 mL) was added. The mixture was slowly warmed up to rt and stirred until completion. The mixture was quenched with an ice-cooled iN HCl solution and extracted with ethyl acetate three times. The combined organic layers were dried and evaporated. The crude product was purified by SiO₂ column (0-15% ethyl acetate in hexanes) to give the desired olefin (1.356 g, 62%) for the following hydrogenation. To a solution of the aforementioned olefin (1.208 g, 3.461 mmol) in THF (30 mL) was added 10 wt % Pd/C (60 mg). The mixture was stirred under a H₂ balloon at rt for 1 h. The mixture was then filtered through celite and the solvent was evaporated under vacuum. The hydrogenation product, ethyl 2,2-dimethyl-11-(5-propylfuran-2-yl)undecanoate, was used in the following hydrolysis without further purification. To a solution of crude ethyl 2,2-dimethyl-11-(5-propylfuran-2-yl)undecanoate (1.131 g,) in THF (24 mL), H₂O (6 mL), and MeOH (6 mL) was added lithium hydroxide (775 mg,). The mixture was stirred at 55° C. for 24 h. The mixture was quenched with an ice-cooled iN HCl solution and extracted with ethyl acetate three times. The combined organic layers were dried, evaporated, and purified by SiO₂ column (0-20% ethyl acetate in hexanes) to give 2,2-dimethyl-11-(5-propylfuran-2-yl)undecanoic acid (665 mg, 64% over two steps). 1H NMR (CDCl₃): δ 0.95 (t, J=7 Hz, 3H), 1.19 (s, 6H), 1.27 (br, 12H), 1.63 (m, 6H), 2.55 (m, 4H), 5.84 (s, 2H); 13C NMR (CDCL₃): δ 13.77, 21.48, 24.85, 24.93, 28.08, 28.14, 29.20, 29.36, 29.46, 29.53, 30.11, 40.55, 42.14, 104.80, 104.95, 154.41, 154.65, 184.94.

### General Experimental Procedures for the Synthesis of Fatty Acid-Amino Acid Conjugates

To a solution of a free fatty acid (1.0 eq) in DMF was added hydroxybenzotriazole hydrate (HOBt, 1.5 eq) and 1-ethyl-3-(3′-dimethylaminopropyl)carbodiimide HCl (EDC-HCl, 1.5 eq) at rt. The mixture was stirred at rt for 1 h before an alkyl ester of an amino acid HCl salt (1.5 eq) and potassium carbonate (3.0 eq) were added. The mixture was stirred at rt for 16 h before quenched with water. The mixture was then extracted with ethyl acetate. The organic layer was washed with water twice. The organic layer was then dried, evaporated, and purified by SiO₂ column (0-30% ethyl acetate in hexanes) to give the purified alkyl ester of fatty acid-amino acid conjugate. To a solution of the aforementioned ester in THP/MeOH/H₂O (4:1:1) was added lithium hydroxide (5.0 eq). The mixture was stirred at rt for 20 h before quenched with 1N HCl solution. The mixture was extracted with ethyl acetate. The organic layer was then washed with water twice. The organics were dried and evaporated to give the corresponding fatty acid-amino acid conjugate. This product can be further purified by SiO₂ column (0-10% MeOH in DCM) if necessary.

Specific examples of the synthesis of fatty acid-amino acid conjugates

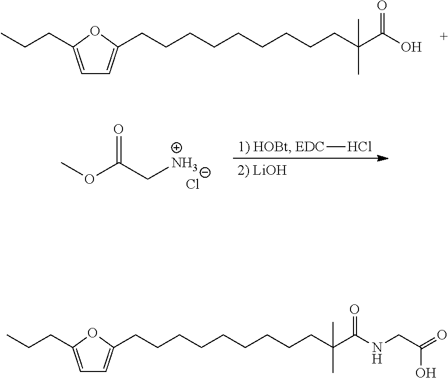

(2,2-Dimethyl-11-(5-propylfuran-2-yl)undecanoyl)glycine

To a solution of 2,2-dimethyl-11-(5-propylfuran-2-yl)undecanoic acid (90 mg, 0.28 mmol) in DMF (5 mL) was added HOBt (57 mg, 0.42 mmol) and EDC-HCl (80 mg, 0.42 mmol). The mixture was stirred at rt for 1 h before methyl glycine HCl (53 mg, 0.42 mmol) and K₂CO₃ (116 mg, 0.84 mmol) were added. The reaction was stirred at rt for 20 h before quenched with water. The mixture was extracted with ethyl acetate and the organic layer was washed with water twice. The organic layer was dried over Na₂SO₄ and evaporated. The crude product was hydrolyzed without further purification. To a solution of the aforementioned crude methyl (2,2-dimethyl-11-(5-propylfuran-2-yl)undecanoyl)glycinate in THF (4 mL), MeOH (1 mL), and H₂O (1 mL) was added lithium hydroxide (34 mg, 1.4 mmol). The mixture was stirred at rt for 20 h before quenched with 1N HCl. The mixture was extracted with ethyl acetate. The organic layer was then washed with water twice. The organics were dried, evaporated, and purified by SiO₂ column (0-10% MeOH in DCM) to give the purified (2,2-Dimethyl-11-(5-propylfuran-2-yl)undecanoyl)glycine (54 mg, 51% over two steps). 1H NMR (CDCl₃): δ 0.94 (t, J=7 Hz, 3H), 1.18 (s, 6H), 1.29 (br, 12H), 1.49 (m, 2H), 1.62 (m, 4H), 2.54 (m, 4H), 4.04 (d, J=5 Hz, 2H), 5.82 (m, 2H), 6.46 (t, J=5 Hz, 1H); 13C NMR (CDCl₃): δ 13.71, 21.45, 24.67, 25.16, 28.05, 28.12, 29.18, 29.32, 29.45, 29.50, 30.08, 41.14, 41.65, 42.15, 104.79, 104.96, 154.37, 154.61, 172.92, 179.37.

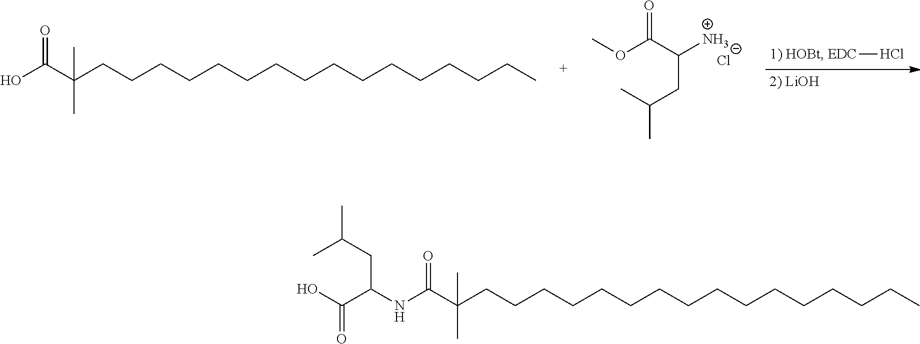

### (2,2-Dimethyloctadecanoyl)leucine

To a solution of 2,2-dimethyloctadecanoic acid (148 mg, 0.474 mmol) in DMF (5 mL) was added HOBt (96 mg, 0.71 mmol) and EDC-HCl (136 mg, 0.71 mmol). The mixture was stirred at rt for 1 h before methyl L-leucine HCl (129 mg, 0.71 mmol) and K₂CO₃ (196 mg, 1.42 mmol) were added. The reaction was stirred at rt for 16 h before quenched with water. The mixture was extracted with ethyl acetate and the organic layer was washed with water twice. The organic layer was dried over Na₂SO₄ and evaporated. The crude product was purified by SiO₂ column (0-20% ethyl acetate in hexanes) to give methyl (2,2-dimethyloctadecanoyl)leucinate (134 mg, 64%) as a white solid. To a solution of methyl (2,2-dimethyloctadecanoyl)leucinate (120 mg, 0.27 mmol) in THF (4 mL), MeOH (1 mL), and H₂O (1 mL) was added lithium hydroxide (33 ng, 1.4 nmnol). The mixture was stirred at rt for 20 h before quenched with 1N HCl. The mixture was extracted with ethyl acetate. The organic layer was then washed with water twice. The organics were dried and evaporated to give (2,2-dimethyloctadecanoyl)leucine (117 mg, 99%). 1H NMR (CDCl₃): δ 0.88 (t, J=7 Hz, 3H), 0.96 (m, 6H), 1.18 (d, J=4 Hz, 6H), 1.25 (br, 28H), 1.49 (m, 2H), 1.6 (m, 1H), 1.72 (m. 2H), 4.58 (m, 1H), 5.97 (d, J=8 Hz, 1H); 13C NMR (CDCl₃): δ 14.08, 21.87, 22.67, 22.83, 24.77, 24.98, 25.25, 25.28, 29.34, 29.54, 29.62, 29.65, 29.66, 29.69, 30.14, 31.91, 40.94, 41.30, 42.17, 50.96, 176.66, 178.73.

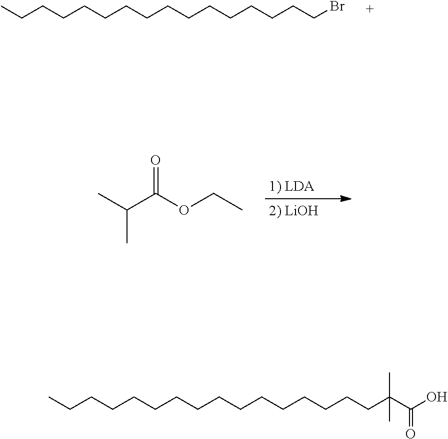

2,2-Dimethyloctadecanoic acid

To a solution of 1-bromohexadecane (1.00 g, 3.28 mmol) and ethyl isobutyrate (0.38 g, 3.28 mmol) in THE (20 mL) was added dropwise lithium diisopropylamide (2M in THF, 2.6 mL, 5.2 mmol) at −78° C. under nitrogen. The mixture was gradually warmed up to rt and stirred overnight before quenched with an ice-cooled 1N HCl aq. solution. The mixture was extracted with ethyl acetate three times. The combined organic layers were dried over NaSO₄ and evaporated under vacuum. The crude product was purified by SiO₂ column (0-10% ethyl acetate in hexanes) to give the desired ethyl 2,2-dimethyloctadecanoate (805 mg, 72%). To a solution of ethyl 2,2-dimethyloctadecanoate (751 mg, 2.21 mmol) in THF (12 mL), H₂O (3 mL), and MeOH (3 mL) was added lithium hydroxide (559 mg, 23.3 mmol). The mixture was stirred at 55° C. for 48 h before being quenched with an ice-cooled iN HCl solution. The mixture was extracted with DCM three times. The combined organic layers were dried, evaporated, and purified by SiO₂ column (0-15% ethyl acetate in hexanes) to give 2,2-dimethyloctadecanoic acid (423 mg, 62%) as a white solid. 1H NMR (CDCl₃): δ 0.88 (t, J=7 Hz, 3H), 1.19 (s, 6H), 1.26 (br, 28H), 1.52 (m, 2H); 13C NMR (CDCl₃): δ 14.12, 22.70, 24.86, 24.94, 29.37, 29.51, 29.64, 29.67, 29.70, 30.12, 31.93, 40.57, 42.12, 184.47.

### General Procedures for the Synthesis of Fatty Acid-Coenzyme a Conjugates

To a solution of a fatty acid (1.0 eq) in DCM was added 1,1′-carbonyldiimidazole (CDI, 2.0 eq) at rt. The reaction was stirred at rt until the free acid was completely consumed (˜2 h). The mixture was then washed with water four times. The organic layer was dried and evaporated. To the resulting oil in THF was added coenzyme A or coenzyme A trilithium salt (0.15 eq) in 0.1M sodium bicarbonate solution (˜5 mL). The mixture was allowed to stir at rt overnight and then acidified to pH˜1 with 1N HCl solution. The crude fatty acid-CoA conjugates were precipitated by the addition of ethyl acetate. The mixture was then filtered, and the solid was washed with acetone and ethyl acetate to give the corresponding purified fatty acid-coA conjugates as white solids.

### Specific Examples of Fatty Acid-CoA Conjugate Synthesis

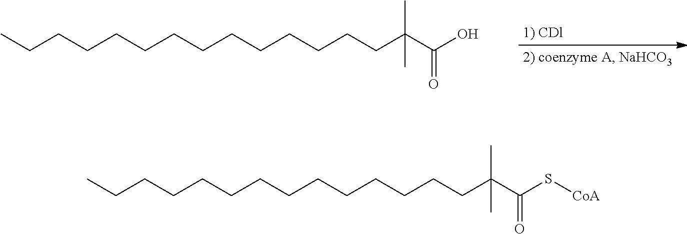

2,2-Dimethyloctadecanoic acid-CoA conjugate

To a solution of 2,2-dimethylhexadecanoic acid (100 mg, 0.352 mmol) in DCM (3 mL) was added 1,1′-carbonyldiimidazole (114 mg, 0.704 mmol) at rt. The reaction was stirred at rt for 2 h. The mixture was then washed with water four times. The organic layer was dried and evaporated. To the resulting oil in THF (5 mL) was added coenzyme A (40 mg, 0.052 mmol) in 0.1M sodium bicarbonate solution (5 mL). The mixture was allowed to stir at rt overnight and then acidified to pH˜1 with 1 N HCl solution. The crude fatty acid-CoA conjugate was precipitated by the addition of ethyl acetate (10 mL). The mixture was then filtered, and the solid was washed with acetone twice and ethyl acetate twice to give the purified 2,2-dimethyloctadecanoic acid-CoA conjugate (12 mg, 22%) as a white solid. 1H NMR (d6-DMSO): δ 0.74 (s, 3H), 0.85 (t, J=7 Hz, 3H), 0.95 (s, 3H), 1.13 (s, 6H), 1.23 (br, 24H), 1.48 (m, 2H), 2.25 (t, J=7 Hz, 2H), 2.85 (t, J=7 Hz, 2H), 3.13 (m, 4H), 3.90 (m, 1H), 4.16 (br, 2H), 4.38 (br, 1H), 4.70 (m, 1H), 4.80 (m, 1H), 5.98 (d, J=6 Hz, 1H), 7.76 (t, J=6 Hz, 1H), 8.12 (t, J=6 Hz, 1H), 8.31 (s, 1H), 8.56 (s, 1H); HRMS: m/z calculated for C39H71N7017P3S⁺ [M+H]⁺: 1034.3840, found 1034.3803.

Synthesis of C18:1-2M-Leu (2,2-dimethyloctadec-9-enoyl)leucine)

To a solution of 7-bromoheptanoic acid (2.08 g, 9.95 mmol) in THF (20 mL) was added borane-THF solution (1M, 21 mL, 21 mmol) at 0° C. The mixture was stirred for 2h while slowly warming up to rt. The reaction mixture was quenched with saturated NaHCO₃ aq. solution and extracted with ethyl acetate. The organic layer was washed with sat. NaHCO₃ aq solution, sat. NaCl aq. solution, and H₂O sequentially. The organic layer was then dried and evaporated to give 7-bromoheptan-1-ol as a colorless oil. To this crude product in dichloromethane (20 mL) was added 1-methylimidazole (2.37 mL, 29.8 mmol), tert-butyldimethylsilyl chloride (1.64 g, 10.9 mmol), and iodine (7.58 g, 29.8 nmmol). Upon the completion of the reaction, it was quenched with sat. Na₂S₂O₃ aq. solution and extracted with dichloromethane three times. The crude product was purified by a silica gel column (0-5% ethyl acetate in hexanes) to give the purified ((7-bromoheptyl)oxy)(tert-butyl)dimethylsilane (1.78 g, 58%).

To a solution of ethyl isobutyrate (2.78 mL, 20.7 mmol) in THF (10 mL) was added Lithium diisopropylamide THE solution(2M, 17 mL, 34 mmol) at −78° C. The mixture was stirred at −78° C. for 15 min and 0° C. for 15 min. The resulting enolate was added slowly into a solution of ((7-bromoheptyl)oxy)(tert-butyl)dimethylsilane (2.13 g, 6.89 mmol) in THE (8 mL) at −78° C. The mixture was warmed up to rt while stirring over 3 h. The reaction was then quenched with sat. NH₄Cl aq. solution and extracted with ethyl acetate 3 times. The combined organic layers were dried and evaporated under vacuum. The crude product was purified by a silica gel column (0-5% ethyl acetate in hexanes) to give ethyl 9-((tert-butyldimethylsilyl)oxy)-2,2-dimethylnonanoate (1.84 g, 77%).

To a solution of 9-((tert-butyldimethylsilyl)oxy)-2,2-dimethylnonanoate (1.84 g, 5.34 mmol) in THE (20 mL) was added tetra-n-butylammonium fluoride THE solution (1M, 8 ml, 8 mmol) at 0° C. The desilylation reaction was stirred at rt until completion. The mixture was quenched with sat. NH₄Cl aq. solution and extracted with ethyl acetate 3 times. The crude product was purified by a silica gel column (0-20% ethyl acetate in hexanes) to give ethyl 9-hydroxy-2,2-dimethylnonanoate (1.07 g, 87%).

To a solution of ethyl 9-hydroxy-2,2-dimethylnonanoate (1.07 g, 4.65 mmol) in dichloromethane (20 mL) was added Dess-Martin periodinane (2.96 g, 6.98 mmol) at rt. Upon the completion of the oxidation, sat. Na₂S₂O₃ aq. solution was added, and the resulting mixture was extracted with diethyl ether 3 times. The combined organic layers were dried and evaporated. The crude product was purified by a silica gel column (0-5% ethyl acetate in hexanes) to give ethyl 2,2-dimethyl-9-oxononanoate (583 mg, 55%).

Nonyltriphenylphosphonium bromide (1.43 g, 3.05 mmol) was dissolved in DMSO (4 mL) and THF (4 mL). To this solution was slowly added KHMDS THF solution (1M, 3 mL, 3 mmol) at 0° C. and stirred for 30 min. The resulting orange ylide solution was added slowly into a solution of ethyl 2,2-dimethyl-9-oxononanoate (583 mg, 2.58 mmol) in THF (4 mL) at −78° C. The mixture was gradually warmed up to 0° C. while stirring. Once the aldehyde was completely consumed based on TLC, the reaction mixture was quenched with an ice cooled NH₄Cl aq. solution and extracted with ethyl acetate 3 times. The combined organic layers were dried and evaporated. The crude product was purified by a silica gel column (0-5% ethyl acetate in hexanes) to give the desired ethyl 2,2-dimethyloctadec-9-enoate (475 mg, 55%).

To a solution of ethyl 2,2-dimethyloctadec-9-enoate (600 mg, 1.78 mmol) in THF (8 mL), MeOH (2 mL), and H₂O (2 mL) was added lithium hydroxide (426 mg, 17.8 mmoL). The mixture was stirred at 55° C. for 48 h. The reaction was then acidified to pH=1 with 1M HCl aq. solution and extracted with ethyl acetate three times. The combined organic layers were dried and evaporated. The crude product was purified by a silica gel column (0-20% ethyl acetate in hexanes) to give the desired 2,2-dimethyloctadec-9-enoic acid or C18:1-2M (538 mg, 98%) as a colorless oil.

To a solution of C18:1-2M (82 mg, 0.26 mmol) in DMF (2 mL) was added HOBt (54 mg, 0.40 mmol) and EDC hydrochloride (76 mg, 0.40 mmol) at rt. The mixture was stirred for 1 h until C18:1-2M was consumed based on TLC. To this solution was then added L-Leucine methyl ester hydrochloride (72 mg, 0.40 mmol) and potassium carbonate (110 mg, 0.797 mmol) at rt. The reaction was stirred for 24 h before quenched with HCl aq. solution (pH=1). The resulting mixture was extracted with ethyl acetate 3 times. The combined organic layers were dried and evaporated. The crude product was purified by a silica gel column (0-20% ethyl acetate in hexanes) to give ethyl (2,2-dimethyloctadec-9-enoyl)leucinate (85 mg, 75%).

To a solution of ethyl (2,2-dimethyloctadec-9-enoyl)leucinate (230 mg, 0.525 mmol) in THF (4 mL), H₂O (1 mL), and MeOH (1 mL) was added LiOH (63 mg, 2.6 mmol) at rt. The reaction was stirred at rt for 2h, and then quenched with HCl aq. solution (pH=1). The mixture was extracted with ethyl acetate three times. The combined organic layers were dried and evaporated. The crude product was purified by a silica gel column (0-10% MeOH in DCM) to give (2,2-dimethyloctadec-9-enoyl)leucine (210 mg, 94%).

### Experimental Procedures

### FuFA Detection by NEM Derivatization

The detection, characterization and quantification of FuFA in biological samples was performed upon derivatization of the furan rings with 100 mM N-Ethylmaleimide (NEM)(Sigma-Aldrich) at 37C for 30 minutes to form their respective Diels-Alder adducts. These adducts were specifically detected monitoring the loss of NEM during the ionization process (m/z:125) upon CID on the triple quadrupole mass spectrometers.

### 11U3-2M Chemical Synthesis

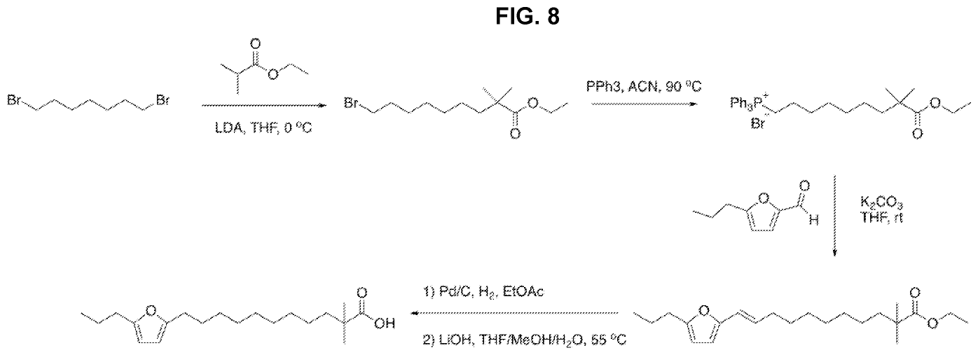

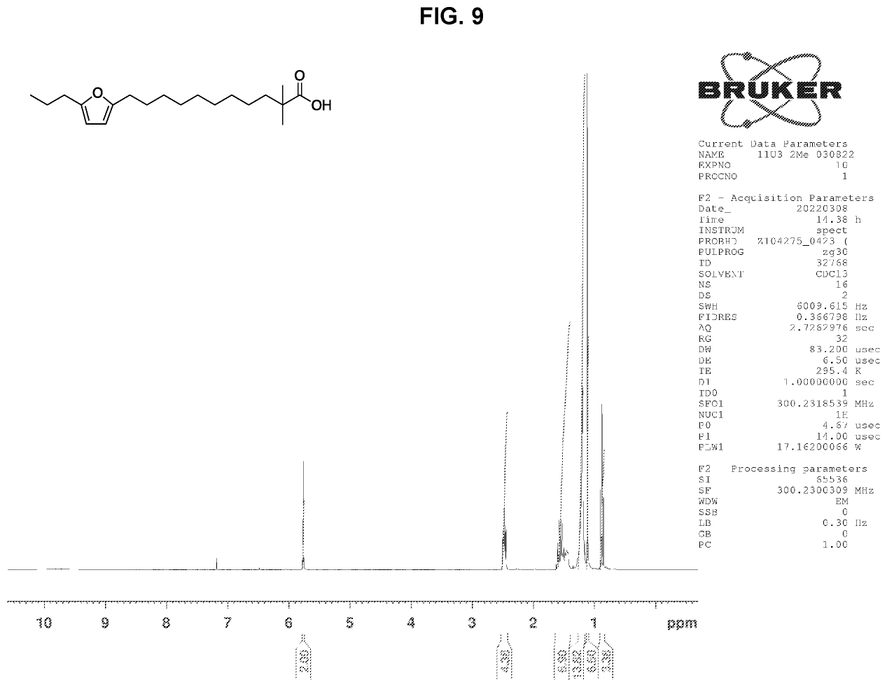

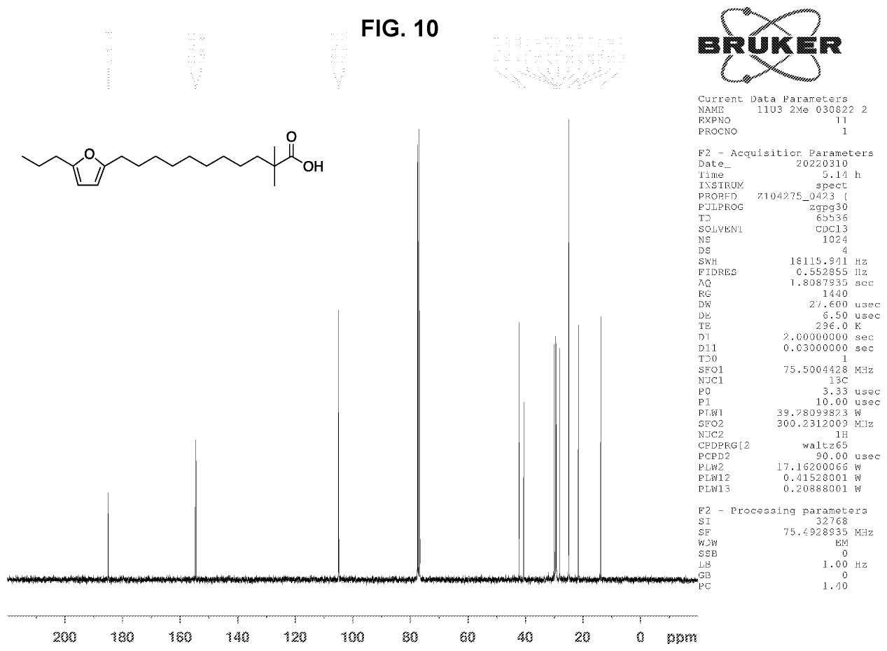

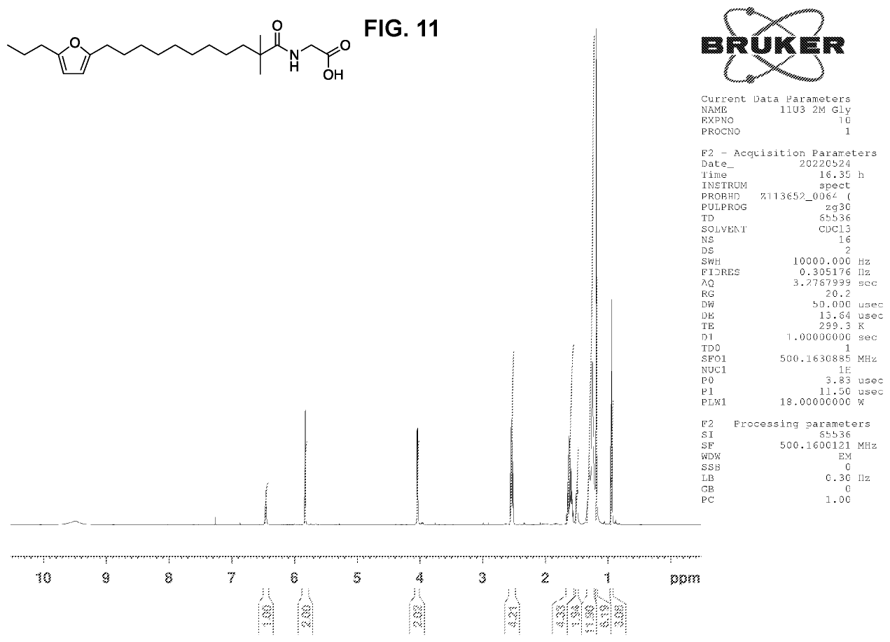

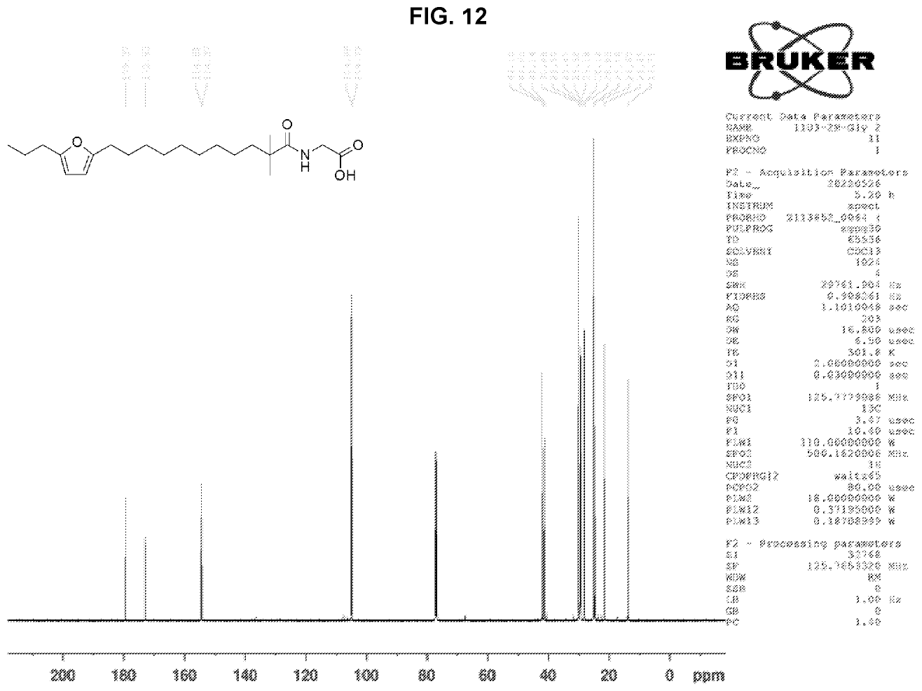

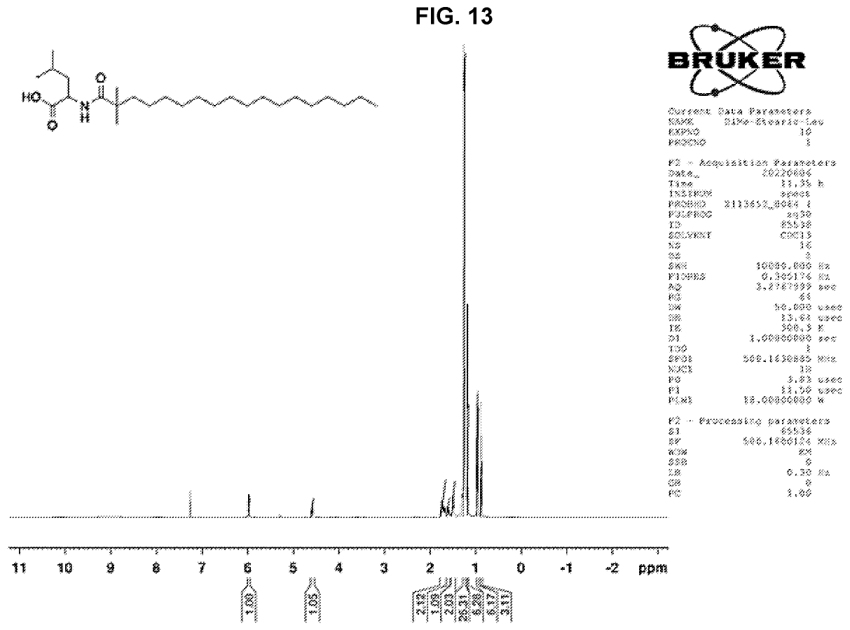

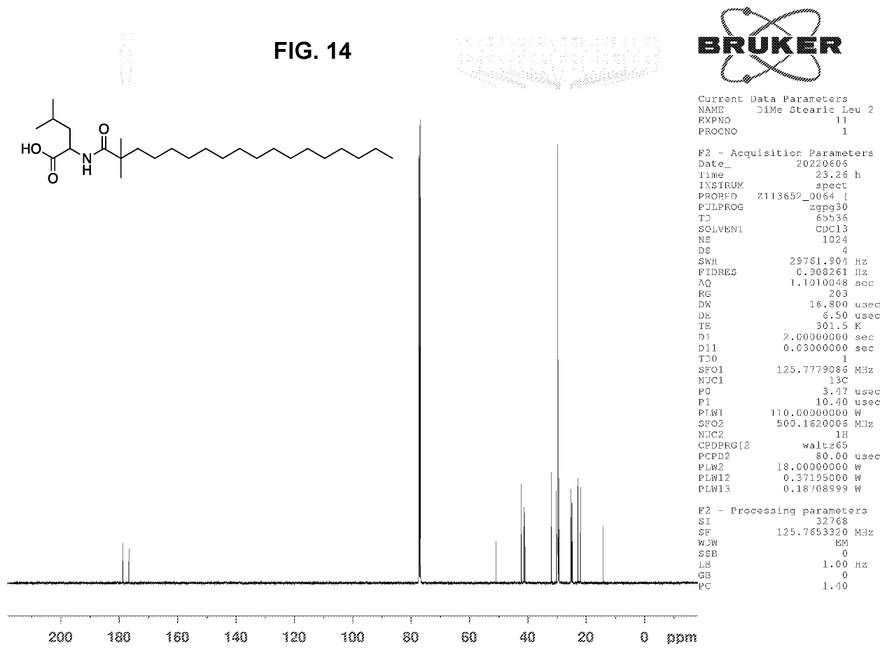

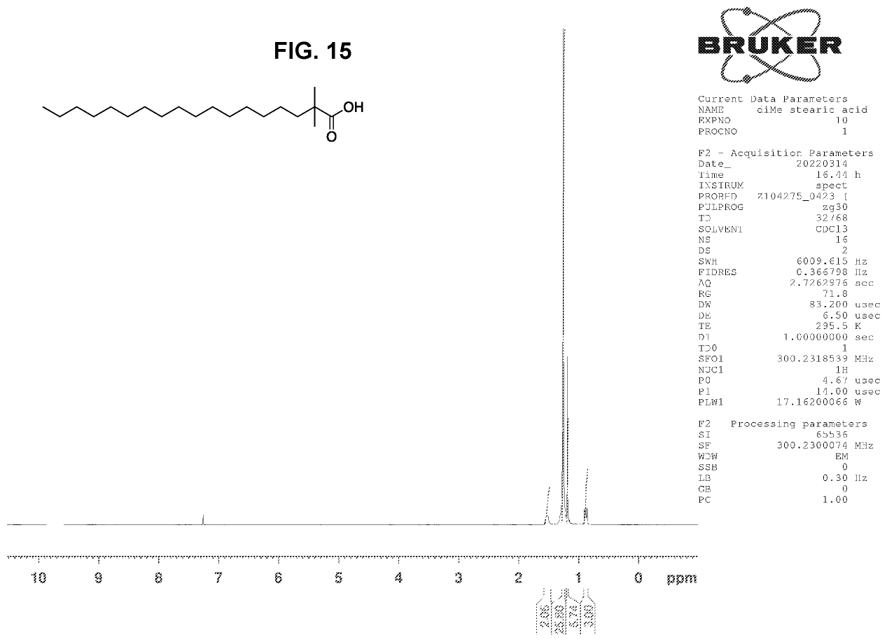

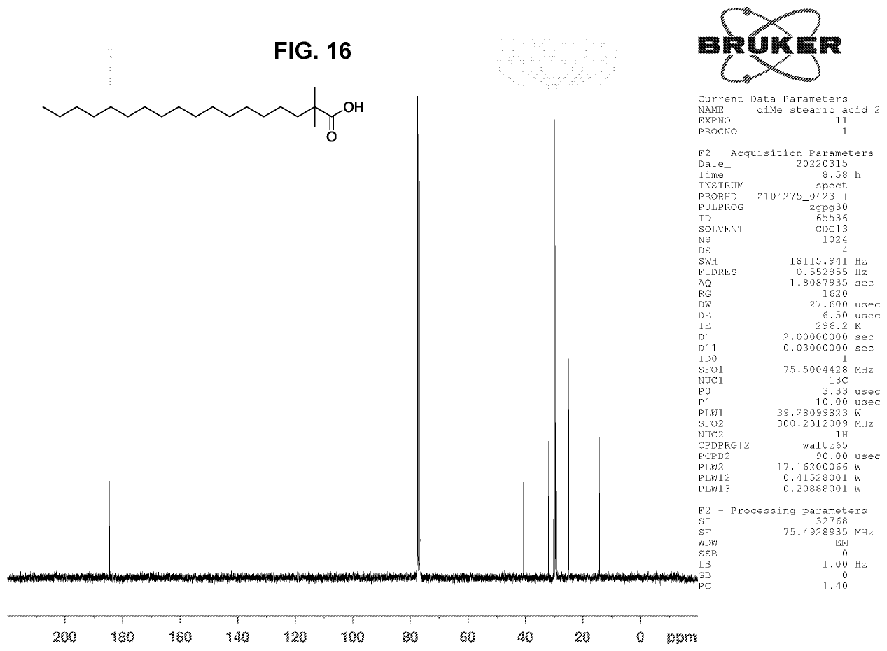

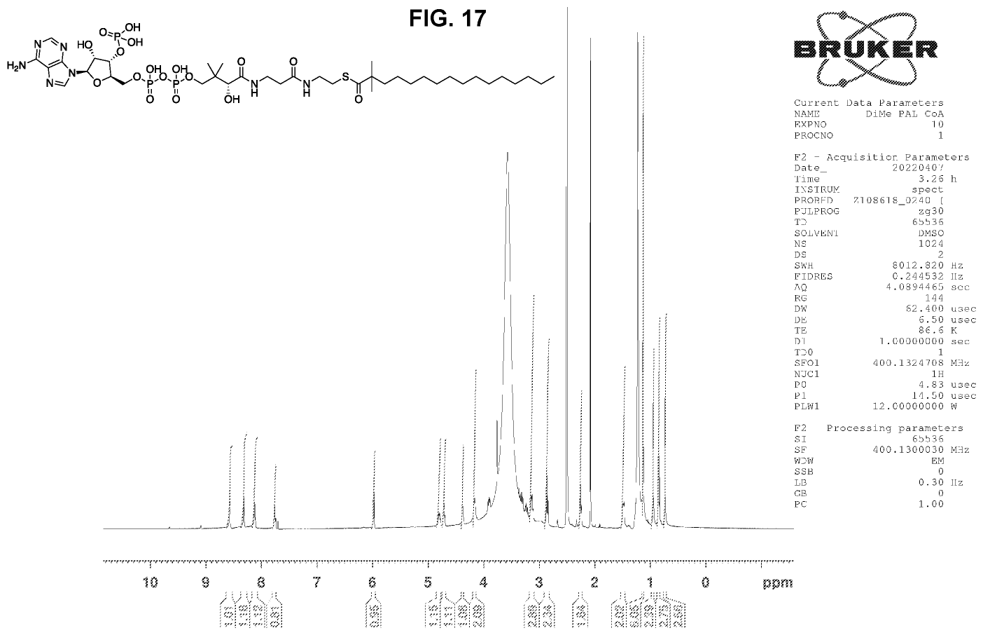

An example of a synthetic scheme for making 11U3-2M is shown in FIG. 8.

### Animals and Experimental Design

Male C57BL/6J mice were purchased from Jackson Laboratory (Bar Harbor, ME, USA). Mice were acclimated for a week before being randomized to receive either a low-fat diet (LFD) or a high-fat diet (HFD) purchased from Research Diets (New Brunswick, NJ, USA). Obesity was induced by the HFD (D12492, with 60% of the adjusted calories derived from fat) for 16 weeks beginning at age of 8 weeks. Age-matched controls were maintained on a LFD (D12450J, matching the sucrose level of D12492). While maintaining the diet, mice under HFD were divided into 4 groups (n=8 per group) and were treated every 2 days by oral gavage for 7 weeks with 100 μl of 11D3 (50 mg/kg), 11U3 (50 mg/kg), or 11U3-2M (50 mg/kg) formulated in a solution of 2:1 of polyethylene glycol (Fisher Scientific, PEG400 #p167-1) and saline, containing 1.5% of octanoic acid (TCI, #00027). Control groups received 100 μl of PEG400/saline/octanoic acid as the vehicle (Veh). LFD mice were divided into 2 groups (n=8 per group) and treated with veh or 11D3 (50 mg/kg) to detect any effect of natural FuFA in LFD. Mice were grouped to start at the same weight mean before starting the treatment. During and before gavage treatment, mice were fed ad libitum and given free access to water. Food intake, water consumption, and mouse weight were monitored every week. At 20 weeks (4 weeks of treatment), a glucose tolerance test (GTT) was performed on all cohorts. After 2 weeks of recovery (22 weeks), a whole-body composition was performed using EchoMRI™-100H. At the end of the 23 weeks study, mice were euthanized with isoflurane followed by laparotomy. The blood was collected, and mice were perfused using 0.9% NaCl. Organs were harvested and snap-frozen in liquid nitrogen gas and stored at −80° C. for further analysis. The Cellerusher (Cell Crusher, Schull, Ireland) tissue pulverizer was used to reduce frozen tissues to a fine, easily recoverable powder using dry ice. For immunohistochemistry, the same liver lobe for every mouse was fixed in Formalin 10% (Fisher Chemical, #SF98-4) for 48h, followed by 75% ethanol baths before paraffin embedding.

A second cohort of mice was set up to confirm the effect of 11U3-2M on mitochondria and fatty acid (FA) β-oxidation using Oroboros analysis in the HFD but also in LFD models. Male C57BL/6J diet-induced obesity (C57BL/6J DIO, #380050) and male C57BL/6J diet-induced obesity control mice (C57BL/6J DIO Control, #380056) were purchased from Jackson Laboratory (Bar Harbor, ME, USA) at 15 weeks of age. Mice were continuously feeding either HFD or LFD during transport and upon reception. Mice were fed for 4 more weeks before starting the treatment. While continuing the HFD (n=8 per group) or LFD (n=8 per group), mice were treated with the same formulation as the first cohort using only 11U3-2M (50 mg/kg) or veh for 11 weeks. Mice were fasted for 5 h and received a single intraperitoneal injection of 50 μl of D31-palmitic acid (Cambridge Isotope, 3 μM, #dlm-215-1) in PEG400/Saline (2:1) and 1.5% of octanoic acid solution 2h before sacrifice in order to study FA metabolism. Blood and tissues were harvested and stored as described above.

### In Vivo Studies

GTT was performed on mice fasted for 5h. Mice were injected intraperitoneally with filter-sterilized 1.5 g/kg glucose in 0.9% NaCl. Blood glucose levels were measured using a hand-held glucometer (Accu-Chek Aviva, Roche Diagnostics, Indianapolis, IN, USA) at 0, 20, 40, 60, 90, and 120 min.

### Plasma Collection and Measurements

After collection, red blood cells and plasma were separated by centrifugation for 5 min at 7500 g at 4° C., aliquoted, and stored at −80° C. Insulin levels were measured by ELISA (Invitrogen, #EMINS) using 50 μl of plasma as per the manufacturer's instructions. Aspartate aminotransferase (AST) and alanine aminotransferase (ALT) levels were both measured using 5 μl of plasma with ELISA (Abcam, #ab263882 and #ab282882, respectively). Metabolic hormones were determined using Luminex xMAP technology (Milliplex mouse metabolic hormone expanded panel #MMHE-44K, Millipore, Burlington, MA, USA). The multiplex metabolic hormones plate was measured as per the manufacturer's instructions.

### Pharmacokinetics Study

Male C57BL/6J (12 weeks old) mice were used to measure the absorption and metabolism of FuFA. In this procedure, I1D3 (50 mg/kg), I 1U3 (50 mg/kg), I1U3-2M (50 mg/kg) and D31-palmitic acid (10 mg/kg) were administered simultaneously (cassette dosing) to single animal. Consequently, the relative pharmacokinetics of multiple compounds can be assessed rapidly with a small number of experimental animals. The compounds were injected either intraperitoneally (n=5) or by gavage (n=5). At 1, 2 and 4h after injection, a small incision on the tail vein was performed and microhematocrit capillary tubes (FisherBrand, #22-362566) were used to collect serial blood samples (50g1 per collection). At 6h, mice were anesthetized, and blood was collected from the vena cava in a terminal blood draw. Blood samples were processed as described above to collect plasma. The pharmacokinetics (PK) of free FuFAs and D31-palmitic acid and their esters found in complex lipids were evaluated using 10 μl of plasma. Briefly, 20 μl of water were added to the 101 of plasma and divided into 2 glass tubes. The sample in one tube was measured as is using GC-MS upon derivatization and the other sample was hydrolyzed before derivatization and quantification. Each sample was spiked with 500 nM of heptadecanoic acid (Sigma, #H3500) and 11U3-CD₃ standards. To obtain the free FA fraction, 200 μl of water was added and quickly vortexed, followed by the addition of 500 μl of ethyl acetate (Fisher, #E195-4). The mixture was vortexed and spun at 1500 g for 5 min at 4° C. The organic layer containing free FA was collected and dried. For the total FA quantification, basic hydrolysis was performed using 500 μl of 1M KOH in MeOH and incubated for 1 h at 60° C. The reaction was quenched by adding 300 μl of 1 M HCl. The mixture was extracted with ethyl acetate (1 mL) and spun at 1500 g for 5 min at 4° C. The organic layer containing total FA was then collected and dried. For MS detection, pentafluorobenzyl bromide (PFB) derivatization was performed using 100 μl of 1% diisopropylethylamine and 100 μl of 2% PFB in acetonitrile (ACN, Fisher #A955) followed by incubation for 30 min at room temperature (RT). Tubes were vortexed, centrifuged at 1500 g for 5 min at 4° C., and dried under nitrogen gas. Samples were resuspended in 100 μl of ACN for analysis by GC.

### Immunohistochemistry

The fixed Liver tissues embedded in paraffin were cut into 4 μm sections. Liver sections were deparaffinized, rehydrated, and stained with hematoxylin-eosin (H&E) for histological evaluation of liver morphology. Quantifications and scores of liver steatosis, ballooning, and inflammation were performed blindly by a pathologist based on 3 parameters—steatosis, inflammation, hepatocyte degeneration.

### RNA Extraction and Quantitative Real-Time PCR

Total RNA was extracted using 50 mg of frozen liver powder. The powder was homogenized in 1 mL of TRIzol solution (Invitrogen, #15-596-026) and 200 μl of chloroform, shaken vigorously for 15 seconds, and incubate at RT for 2-3 min. Lysates were centrifuged at 12000 g for 15 min at 4° C. The upper aqueous phase was transferred in a new ice-cold tube and diluted with 500 μl of ice-cold isopropyl alcohol and incubate at RT for 10 min. Tubes were centrifuged at 12000 g for 10 min at 4° C. The RNA pellet was washed twice using ice-cold 75% ethanol and centrifugation for 5 min at 7500 g at 4° C. The RNA pellet was dissolved in RNase-free H₂O and the concentration was measured using Nanodrop 2000 (Thermo Fisher Scientific). All RNA samples had an absorbance of 260 nm/280 nm ratio between 1.95-2. The RNA sample (1 μg) was subjected to reverse transcription with the iScript cDNA synthesis kit. The TaqMan gene expression assays-on-demand system (Applied Biosystems, Foster City, CA, USA) was used for gene expression assessment by quantitative PCR (qPCR) using GAPDH as housekeeping gene using the comparative Ct method.

Bioinformatics and Biostatistics Analyses on RNA Sequencing Data Transcriptome sequencing was performed on mice samples from each group with four replicates per condition. Quality control was first performed on the raw sequencing data by tool FastQC. Then low-quality reads and adapter sequences were trimmed out by tool Trimmomatic. After pre-processing, the surviving reads were aligned into mouse reference genome mm10 by STAR aligner. Gene counts per gene per sample were quantified by function—quantMode GeneCounts supported by the STAR aligner. Gene expression normalization was performed by R package DEseq2.

Given the gene expression profile, differential expression analyses were performed based on gene counts by DEseq2 package. Five pairwise comparisons were analyzed: LFD+Veh vs LFD+11D3, HFD+Veh vs HFD+11D3, HFD+Veh vs HFD+11U3, HFD+Veh vs HFD+11U3-2M and LFD+Veh vs HFD+Veh. Differentially expressed genes (DEGs) were defined by FDR=5% and absolute fold-change equal to or greater than 1.5. For the downstream analysis, Ingenuity Pathways Analysis (IPA) was employed to perform pathway enrichment analysis on the DEGs. Significant pathways were defined by FDR=5%. Regulation directions were measured by Z-scores, where a positive Z-score indicates activation of the pathway and a negative Z-score means inhibition, while some pathways show out unknown or mix of regulation directions.

### Fluorescence RNA In-Situ Hybridization

Fluorescence RNA in-situ hybridization was performed on tissue sections using LNA ISH Optimization Kits (Qiagen, #339459) according to manufacturer's instructions. Briefly, the sectioning slides were deparaffinized and digested by proteinase K approach for 15 min at 37° C. U6, scrambled RNA, and GM15441 (LCD0173184) probes were hybridized at final concentration of 1 nM, 40 nM and 40 nM, respectively, followed by stringent washes of serial diluted SSC (Sigma, #S6639-1 L). After 30 min blocking, the slides were incubated with anti-DIG-POD (Roche, #11207733910) at 1:400 dilution for 60 min at RT according to manufacturer's instructions. TSA-plus Cyanine 3 (Akoya Bioscience, #NEL744001KT) substrate was added to the sections and incubated twice (5 min per incubation) to amplify the fluorescence signal. NucBlue Live ReadyProbes Reagent was applied to the slides at last to facilitate cell nuclei staining. Images were acquired on a fluorescent microscope (Leica, DMi8) using the Ocular Advanced Scientific Camera Control software (Digital Optics Limited).

### Western Blot

The liver samples (50 mg) were homogenized and vortexed in ice-cold RIPA buffer containing phosphatase (Fisher Scientific, #PIA32957) and protease (Fisher Scientific, #PIA32953) inhibitor cocktails. Protein concentration was determined using a BSA assay (Pierce). Protein lysates were mixed with LDS Sample Buffer (1:4) and heated for 2 min at 95° C. The lysates were then loaded and electrophoresed on a 10% Tris-Glycine eXtended (BioRad, Criterion #5671034). The proteins were electrophoretically transferred to a PVDF membrane (BioRad, #1620177), and the transfer was confirmed by staining the membrane with 0.1% Ponceau S solution (Sigma-Aldrich, #6226-79-5). The membrane was then blocked for 90 min at RT with 5% nonfat dry milk (LabScientific) or 5% BSA (MilliporeSigma) in TBS-T (tris buffered saline+0.1% Tween 20 [Thermo Fisher Scientific]). After blocking, the membrane was incubated overnight at 4° C. with primary antibodies: anti-Acetyl-CoA Carboxylase (anti-ACC, Cell Signaling, #3662, 1/1000), anti-ATP citrate lyase (anti-ACLY, Cell Signaling, #4332, 1/1000), anti-β-ACTIN (Cell Signaling, #4970, 1/1000). The Blots were rinsed three times for 15 min with TBS-T for and incubated with appropriate HRP-conjugated anti-rabbit (Cell Signaling, #7074, 1/10000) for 90 min at RT. The Membranes were washed three times for 15 min in TBS-T and visualized with Western Lightning Plus-ECL, Enhanced Chemiluminescence Substrate kit (Perkin Elmer LLC, #50-904-9323) or SuperSignal™ West Femto Maximum Sensitivity Substrate (Bio-Rad, #34094) and ChemiDoc imager (Bio-Rad). Quantitative densitometric analysis was performed using Image Lab software (Bio-Rad).

Thermal Shift Assay Proteins were extracted from the liver powder (50 mg) with 1 mL of phosphate-buffered saline (PBS—Gibco, Paisley, UK) containing phosphatase (Fisher Scientific, #PIA32957) and protease (Fisher Scientific, #PIA32953) inhibitors. The suspension was vortexed and centrifuged for 20 min at 16000 g at 4° C. and the supernatant was collected. Aliquots of 40 μl were transferred to MicroAmp 8-tubes strip (Applied Biosystems, #43-582-93) and heated at different temperatures (37; 45; 47; 49; 50.5; 52; 53.5; 55° C.) using the StepOne PCR system (Applied Biosystems). The tubes were centrifuged for 20 min at 16000 g at 4° C. to remove the denatured proteins. The Supernatants were transferred to new tubes and analyzed by western blot as described above.

Mouse Primary Hepatocytes Isolation Primary mouse hepatocytes were isolated from 8-week-old male C57BL/6J mice from Jackson Laboratories using a two-step collagenase perfusion. Mice were anesthetized using Isoflurane (Piramal Critical Care, Andhra Pradesh, India). The abdominal area fur was cleaned with 70% alcohol and a U-shape incision was performed to expose internal organs. The portal vein was cannulated with an i.v. catheter (24-gauge×3/4″; Terumo Medical Corporation, Elkton, MD). The catheter was secured and maintained with a surgical knot using Ethilon nylon monofilament 7-0 (Ethic, Johnson and Johnson, USA). The infusion tubing connected to a pump was inserted into the catheter, and the liver was perfused at a rate of 5 ml/min with 50 ml of pre-warm (40° C.) liver perfusion media (Gibco, #17701038) supplemented with 2% penicillin-streptomycin (Sigma Chemical Company). The vena cava was cut right after the beginning of the perfusion to allow the outflow. The liver was then perfused at the same rate with 50 ml of pre-warm (40° C.) liver digest media (Gibco, #17703034). During the whole perfusion process, occlusion of the vena cava was performed every 2 min for a few seconds to create backpressure and liver swelling to allow good liver perfusion. At the end of the perfusion, the liver was removed and placed in a 100 mm dish filled with cold 20 ml of plating media composed of William's E Medium (WEM) supplemented with primary hepatocytes plating supplements (Gibco, #CM3000) according to the manufacturer's instruction. The digest liver was torn with forceps to obtain hepatocytes suspension. The hepatocytes were filtered into 50 ml centrifuge tubes through a 100 um nylon cell strainer (Falcon, Durham, NC) to remove the undigested debris. The filtrate was centrifuged for 3 min at 50×g at 4° C. to pellet the hepatocytes. The hepatocytes were washed 2 times with plating media and centrifuge to obtain a pellet. Hepatocytes were re-suspended into 20 ml of plating media, mixed with 20 ml of 40% cold percoll (GE Healthcare, Uppsala, Sweden), and centrifuge for 7 min at 150×g at 4° C. The supernatant and dead cells were discarded, and the bottom phase was washed 2 times and centrifuge for 3 min at 50×g at 4° C. Hepatocytes were resuspended in 10 ml of plating media and counted using a hemacytometer (Fisher Scientific). Cell viability (>90%) was assessed using Trypan blue stain (Gibco, Grand Island, NY). Hepatocytes were seeded in collagen-coated plates (Gibco, Grand Island, NY) at 2.2×10⁵ cells/6-well. After a 3h attachment period in a 37° C. incubator with 5% CO₂, the medium with unattached cells was removed and cells were washed with PBS (Gibco, Paisley, UK). Fresh maintenance medium composed of WEM supplemented with primary hepatocytes maintenance supplements (Gibco, #CM4000) according to manufacturer's instruction was added. Hepatocytes are easily distinguished from other non-parenchymal cell types because of their larger size. Hepatocytes were cultured for approximately 16h in a 37° C. incubator with 5% CO₂ before experimental procedures.

### HPLC-MS Analysis of Cholesterol, Cholesterol-Ester and Triglycerides.

Cholesterol, total cholesterol-ester (TCE) and triglycerides (TG) were extracted from plasma sample. Briefly, 10 μl of plasma was used for the extraction using 2001 of ethyl acetate in the presence of internal standard for cholesterol (Cholesterol-d7; Avanti Polar Lipids, Inc. #700041), TCE (16:0 cholesteryl-d7 ester; Avanti Polar Lipids, Inc. #700149) at 500 nM, and TG (triheptadecanoin, Nu-Chek Prep., Inc.) at 500 pM, vortexed and spined 5 min at 1.500 g at 4° C. For cholesterol and TCE, 30 μl of the supernatant was mixed with 701 of ACN and ran by LC-MS, following the specific MRM transitions (369.3/147.3 and 376.3/147.3 for Cholesterol or Cholesteryl esters and d7-Cholesterol or d7-cholesteryl ester, respectively), after in source neutral losses of FA from cholesterol or TCE.

Plasma TG were analyzed by HPLC-HR-MS/MS using a C8 Luna column (2×150 mm, 5 μm, Phenomenex) with a flow rate of 0.4 mil/min and mobile phases of acetonitrile/water (9:1, v/v) 0.1% ammonium acetate (solvent A) and isopropanol/acetonitrile 0.1% ammonium acetate (7:3, v/v) (solvent B). The gradient program was the following: 35%-100% solvent B (0.1-10 minutes), 100% solvent B (10-13 minutes), followed by 4 minutes of re-equilibration to initial conditions. A Q-Exactive hybrid quadrupole-orbitrap mass spectrometer (ThermoFisher) was used in positive mode with the following parameters: auxiliary gas heater temperature 250° C., capillary temperature 300° C., sheath gas flow rate 20, auxiliary gas flow rate 20, sweep gas flow rate 0, spray voltage 4 kV, S-lens RF level 60 (%). Full mass scan analysis ranged from 700 to 1500 m/z at 17500 resolutions.

### FuFA-CoA Detection and Analysis by Mass Spectrometry

### Liver

Liver powder (50 mg) was used and dissolved in plastic Eppendorf with 1001 of milli-Q purified water and vortexed well. Then, 200 μl of ACN containing 400 nM n-Heptadecanoyl coenzyme A lithium salt as internal standard (C17-CoA standard, NuCheck) was added, vortexed thoroughly, and centrifuged for 4 min at 10000 g at 4° C. For protein precipitation, the supernatant was transferred to a new tube and 20 μl was diluted with 180 μl of ACN/water (4:1 ratio) in clean mass spectrometry vials for analysis of CoA's.

### Primary Hepatocytes

Cells were isolated and seeded in 6-well plates as described above. Cells were treated for 1 h with 11D3 (50 μM), 11U3 (50 μM), or 11U3-2M (50RM) complexed with FA free bovine serum albumin (Sigma, #A7511). After an hour, cells were washed 3 times with PBS, and WEM media was added to the wells. After 0, 15, 45, and 90 min of new media, cells were washed, scraped, harvested with PBS, and centrifuged for 4 min at 10000 g at 4° C. PBS was discarded and the pellet was resuspended in 150 μl of ice-cold methanol (MeOH, LC-MS grade, Alfa Aesar #UN1230) containing C17-CoA standard (400 nM), vortexed, and centrifuged for 4 min at 10000 g at 4° C. MeOH was transferred to clean mass spectrometry vials for analysis.

FuFA-CoA in liver and primary hepatocytes were detected with an API 5000™ mass spectrometer after chromatographic separation using a 150×2 mm Luna® 5 μm C8 100 C8 Å LC column (OOF-4040-BO) with 5% solvent B (ACN, 0.1% ammonium) at a flow rate of 0.35 ml/min using a system solvent started in 5% solvent B (ACN, 0.1% ammonium hydroxide) and 95% of solvent A(water+0.1% ammonium hydroxide) for 30 seconds, followed by an increase from 5-65% gradient of solvent B over 6 min, followed by re equilibrate condition. Quantitation of analyte and internal standard peak areas was determined using the Quantitation Wizard in Sciex's Analyst® Software.

Untargeted/targeted metabolomic mass spectrometry

### Untargeted

Liver powder (50 mg) was resuspended in 500 μL of H2O and extracted using ethyl acetate (1 mL). Phases were separated by centrifugation for 5 min at 1500 g at 4° C. and the organic phase was collected and dried under nitrogen gas. For acetonitrile precipitation, 200 μL of ice-cold ACN was added to liver tissue homogenized in water and vortexed thoroughly. Samples were centrifuged for 5 min at 1500 g at 4° C. and all the phases were removed and dried under nitrogen gas. Dried samples were reconstituted with McOH and transferred to clean mass spectrometry vials for analysis.

Mass spectrometry data was collected using a Q Exactive Orbitrap (ThermoFisher Scientific) to obtain high-resolution accurate mass data in positive and negative ion modes. Two different Chromatography system were used: The organic phase from Bligh and Dyer extraction sample was performed using a 100×2 mm Luna® 5 μm C18(2) column (phenomenex,00D-4252-BO) with a 0-100% gradient of solvent B (90:10 isopropyl alcohol, ACS reagent for HPLC (Honeywell #AH323):ACN, 0.1% formic acid, reagent grade (Sigma #F0507) and solvent A (70:30 Water, Optima™ LC/MS Grade (Fisher #W6500):acetonitrile, 0.1% formic acid) over 20 min, followed by 100% solvent B for 6 min before a return to 0% solvent B for 4 min at a flow rate of 0.650 mL/min. Chromatography for aqueous Bligh and Dyer phase and acetonitrile precipitation extract was performed using the same column with a gradient of 10-100% solvent D (ACN, 0.1% formic acid) and solvent C (water, 0.1% formic acid) over 18 min, followed by 100% solvent D for 6 min before returning to 10% solvent D for 6 min at a flow rate of 0.650 mL/minute. Identification of compounds was running by Compound Discoverer 3.2 LCMS analysis software.

### Targeted Ceramide and Carnitine Conjugate Evaluation

Ceramide and carnitine conjugates were extracted in the presence of ceramide standard (18:1/17:0) (N-heptadecanoyl-D-erythro-sphingosine, Avanti Polar Lipids #860517) using ethyl acetate and quantified using mass spectrometry.

Mass spectrometry data was collected using an API 5000 LC/MS/MS in positive ion mode. Chromatography for organic phase Bligh and Dyer sample extract was performed using the same LC column with 30% solvent B for 30 seconds, followed by a 30-100% gradient of solvent B in solvent A over 9 min, followed by 100% solvent B for 3 min before a return to 0% solvent B for 3 min at a flow of 0.600 mL/minute. Quantitation of analyte and internal standard peak areas was determined using the Quantitation Wizard in Sciex's Analyst® Software.

### Statistical Analysis

Results are expressed as mean±SEM. Statistical analysis was performed with GraphPad Prism, and the data were analyzed by one-way or two-way analysis of variance (ANOVA) with Holm-Šídák multiple comparison post hoc comparisons and Student's t-test. Data normality was measured using 4 normality tests (D'Agostino-Pearson, Anderson-Darling, Shapiro-Wilk, and Kolmogorov-Smirnov). All results were considered significant at p<0.05. Statistical significance is stated as follows: *p<0.05, **p<0.01, ***p<0.001, ****p<0.0001.

### Results

### Detection and Characterization of Natural Furan Fatty Acids.

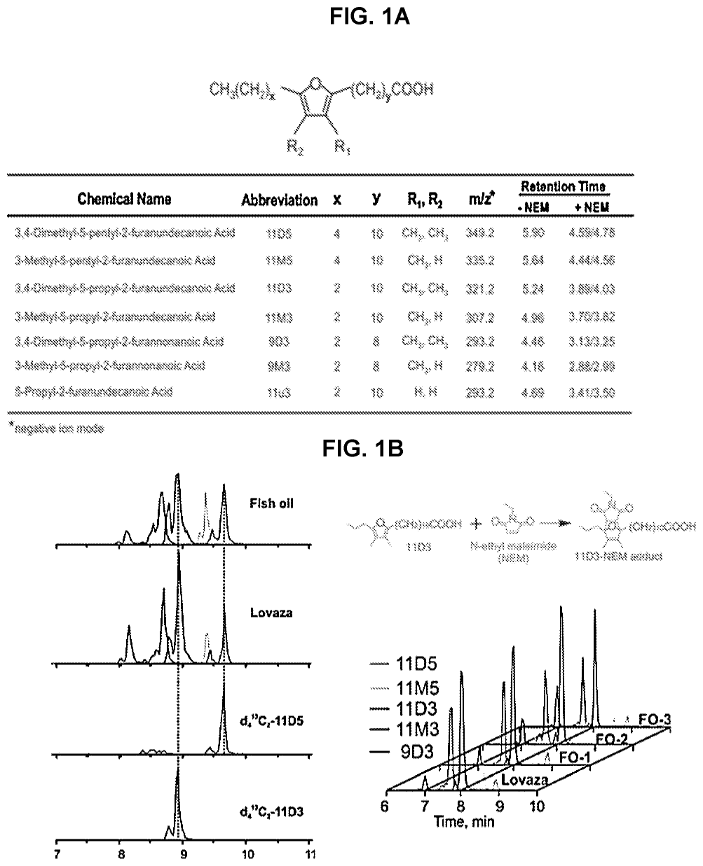

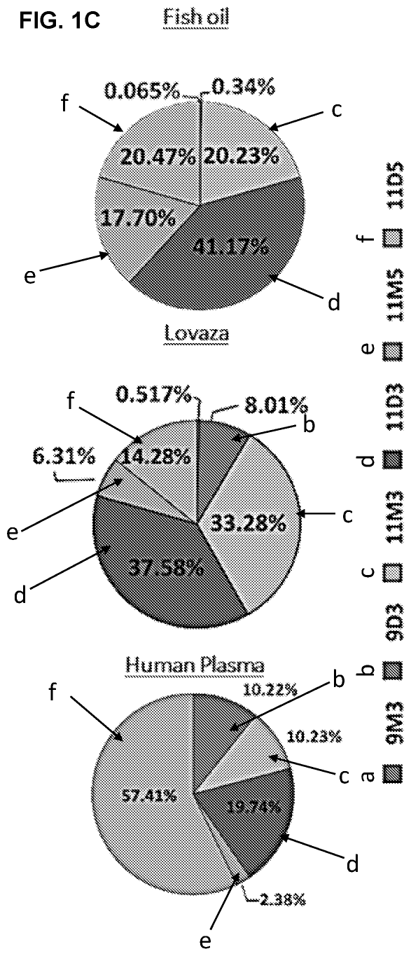

FuFA can undergo oxidation, reduction, and metabolism, and therefore multiple structures can be found in plants and in fish, leading to various nomenclatures. For example, one of the natural FuFA (11D3) is named as follows: “11” stands for the number of carbons on the carboxyalkyl chain, “D” represents the two methyl groups attached at β-positions (One methyl will be represented by a “M”, no methyl will be represented with a “U”) on the furan ring and “3” represents the number of carbons on the alkyl chains (FIG. 1A).

By using a NEM-derivatization, we have been able to detect different FuFA in FO, Lovaza™ medication and plasma of human treated with Lovaza™ (FIG. 1B). FIG. 1C represent the percentage of FuFA molecules present in FO, Lovaza™ medication and plasma of human treated with Lovaza™ showing a higher concentration of FuFA with two methyl groups. 11 D3 and 11U3 are two closely similar FuFA at the exception of that 11U3 is lacking two methyl groups on the furan ring which slows its degradation and prevents its metabolization into CMPF.

Chemical synthesis of dimethyl 11U3 (11U3-2M) increases its stability and reduces its incorporation in fatty acids.

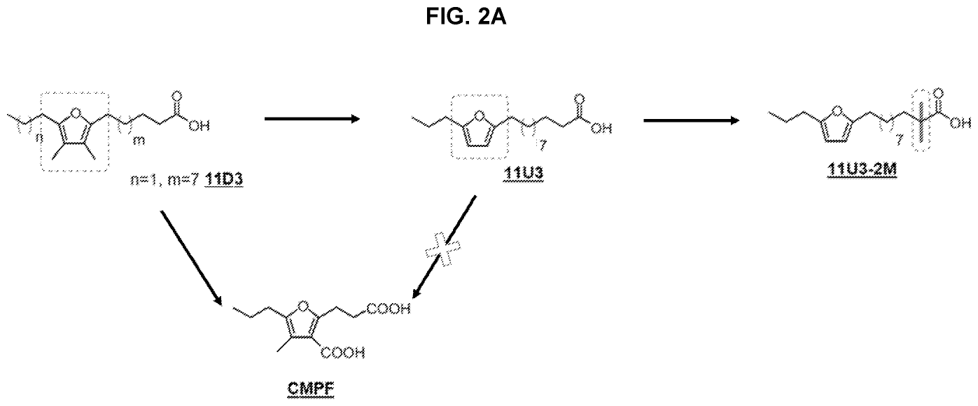

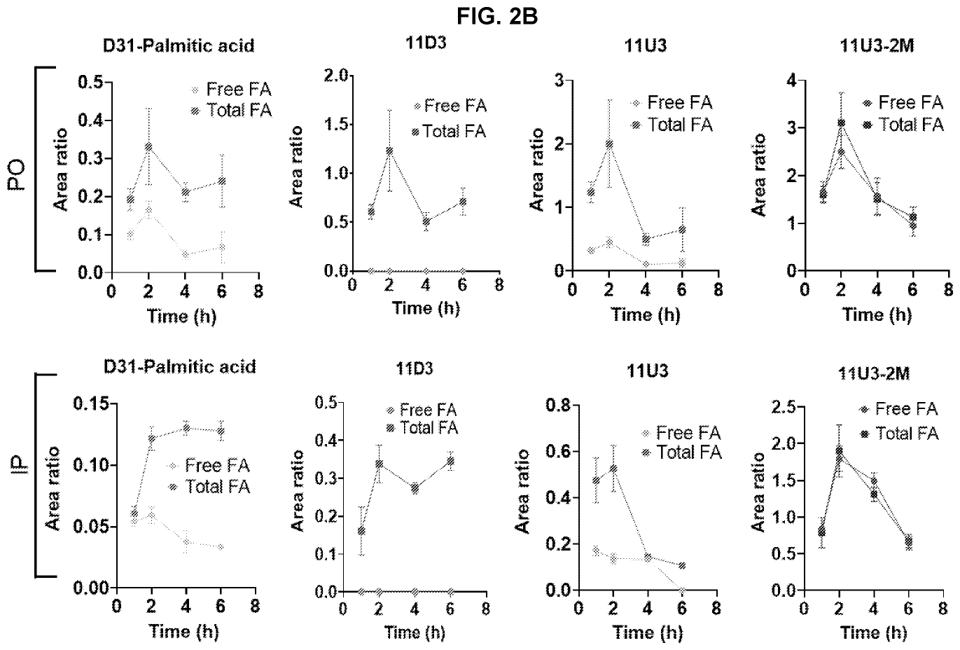

The structurally engineered 11U3-2M has been chemically synthetized to present two methyl groups on the carbon adjacent to the carboxylic group. This modification prevents the oxidation and enzymatic metabolism that other natural FuFAs (i.e., 11D3) undergo leading to CMPF as final product (FIG. 2A).

To confirm the increased stability and distinctive absorption pathway, a cassette pharmacokinetic (PK) study of 11U3-2M in comparison to 11D3 and 11U3 was performed in mice. All the compounds were injected either by gavage (PO) or intraperitoneal injection (IP). D31-palmitic acid was used as the reference FA. Overall, 11D3, 11U3, and D31-palmitic acid were rapidly esterified. On the contrary, 11U3-2M was present as free acid with little to none incorporation in phospholipids or triglycerides. Additionally, as expected 11U3-2M demonstrated greater oral stability compared with natural FuFAs (e.g. 11D3) as longer half-lives were observed. It is known that long-chain fatty acids are distributed in chylomicrons upon re-esterification through lymph vessels, bypassing the initial hepatic transit via the portal vein. The resistance to the esterification of 11U3-2M into TG and absence of incorporation into chylomicrons shows that 11U3-2M is absorbed through the hepatic portal vein, leading to liver-targeting therapeutic effects. (FIG. 2B).

### 11U3-2M Reverses Steatosis; and Improves Glucose Clearance and Insulin Resistance

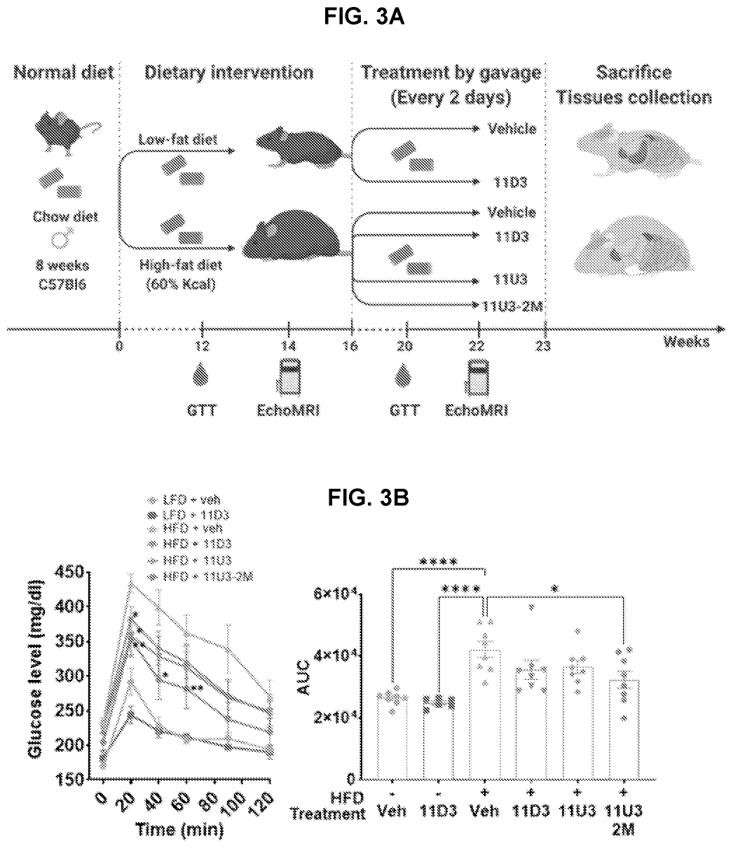

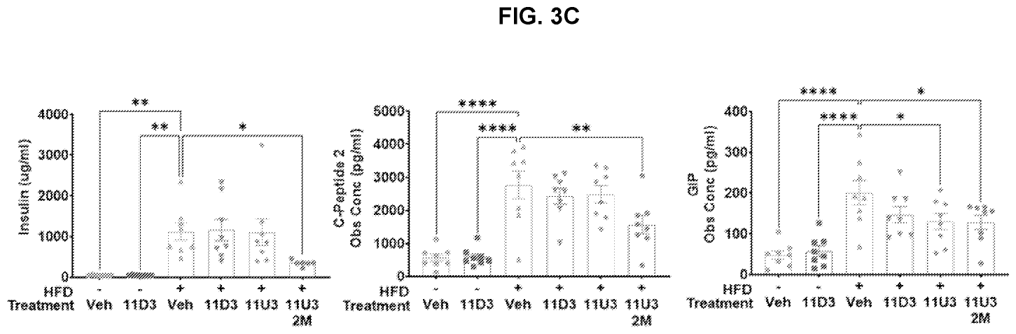

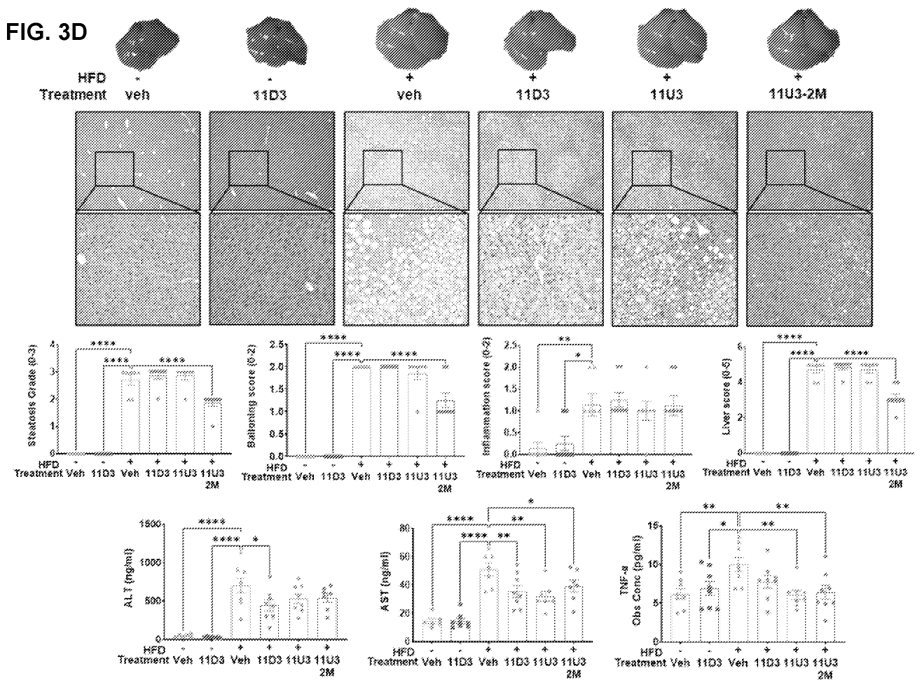

After the characterization and confirmation that 11U3-2M was more stable and had a longer effect after injection, we tested these three FuFAs (11D3, 11U3, and 11U3-2M) in a NAFLD mouse model (FIG. 3A).

The effects of FuFA treatment on glucose metabolism, insulin resistance and steatosis were analyzed using a HFD model in C57BL/6J mice. As shown in FIG. 3B, 11U3-2M significantly improved blood glucose clearance measured by GTT compared to veh treatment, after 4 weeks of treatment. The GTT showed 18% (p<0.01), 26% (p<0.05) and 22% (p<0.01) less glucose at 20-, 40- and 60-min time point, respectively, in 11U3-2M vs veh treated mice. Fasting blood glucose showed a trend in decreased glucose in 11U3-2M treated mice, and the area under the curve from 0 to 120 min showed a 24% decrease in glucose in 11U3-2M treatment compared to veh (p<0.05, FIG. 3B). No significant difference was observed in 11D3 and 11U3 treatment compared to veh.

In line with glucose metabolism, 11U3-2M treated mice presented a lower insulin plasma level measured by ELISA compared to veh (68%, p<0.05, FIG. 3C). A milliplex analysis of mouse metabolic hormones was used to find any hormones that were correlated with insulin level secretion. C-peptide 2 and gastric inhibitory polypeptide (GIP), two hormones known to have a role in insulin secretion, were significantly lower in plasma from mice treated with 11U3-2M compared to veh (43%, p<0.01 and 36%, p<0.05, respectively. FIG. 3C).

Mice fed with LFD presented hepatocytes with clear structure without inflammation, while mice in HFD+veh group showed greater steatosis accompanied by ballooning degeneration of hepatocytes (p<0.0001 for both), which was significantly alleviated in mice treated with 11U3-2M (p<0.0001 for both). No difference was observed in 11D3 and 11U3-treated mice. Despite an obvious inflammation was detected in the HFD+veh group compared to LFD groups, no difference was observed in the three FuFA-treated groups (FIG. 3D). However, milliplex analysis of mouse metabolic hormones showed a significant reduction in plasma levels of AST and TNF-α in 11U3-2M-treated group (FIG. 3D).

These results show a strong effect of 11U3-2M in the protection of liver NAFLD with reduced glucose, insulin, and insulin-related hormones with a concomitant preservation of liver structure and function.

11U3-2M Decreases Plasma Levels of Cholesterol-Ester and Triglycerides and Increases Metabolic Changes with a Major Effect on Sphingolipids and Acylcarnitine in Liver

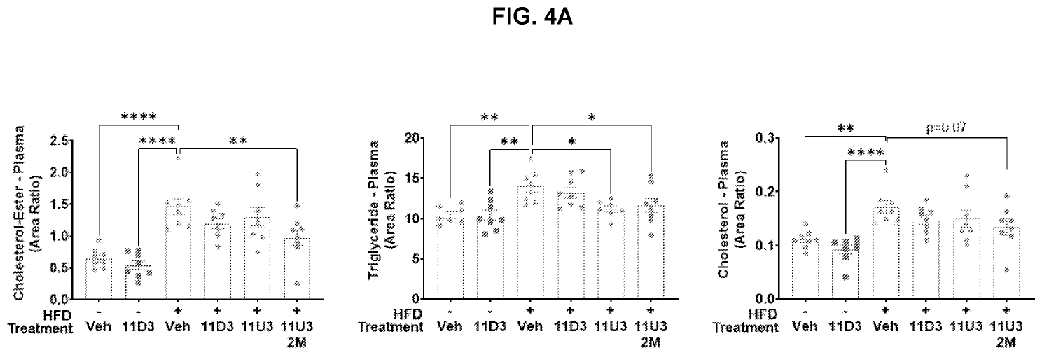

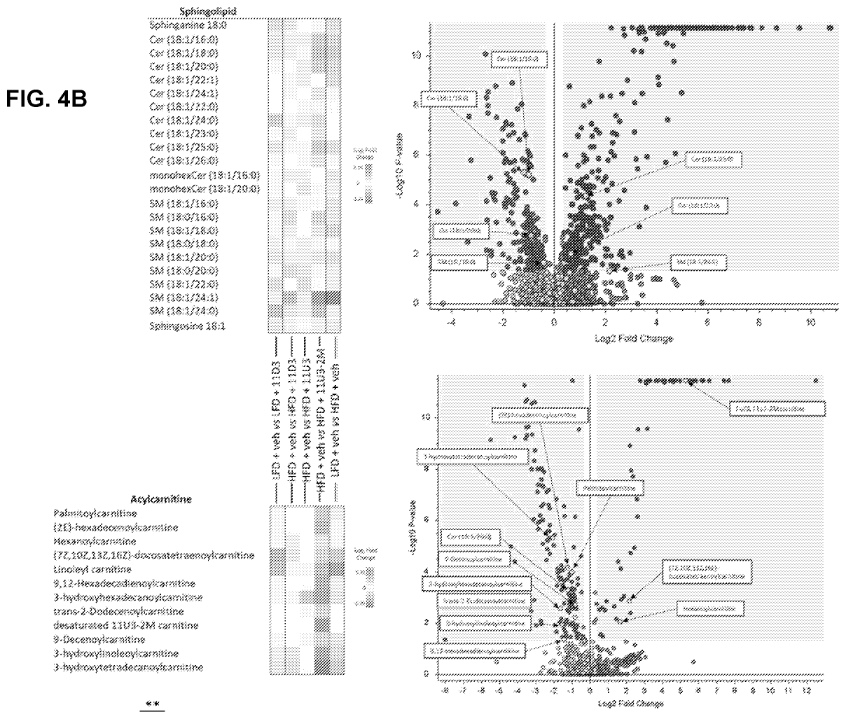

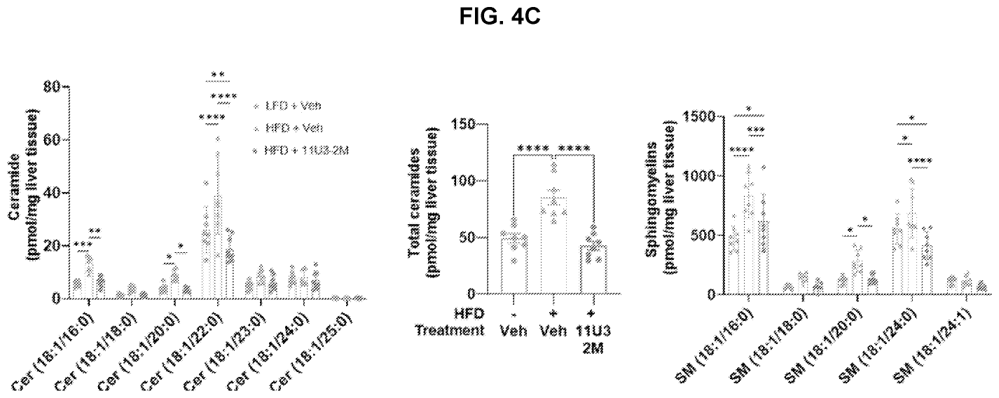

Mass spectrometry analysis of plasma showed significant increase of triglycerides (TC, p<0.01), total cholesterol-ester (TCE, p<0.0001) and total cholesterol (p<0.01) in HFD+veh vs LFD+veh. Both TCE and TG were reduced by 11U3-2M treatment (33%, p<0.01 and 16%, p<0.05; respectively) in HFD (FIG. 4A) with a reduction in TC in 11U3-2M compared to HFD+veh group (FIG. 4A).

Untargeted metabolomic analysis on liver was performed to discover metabolites regulated by FuFA treatment. Of note, most compounds that showed a significant increase with HFD showed a corresponding decrease following 11U3-2M treatment and vice versa (FIG. 4B), indicating that the 11U3-2M treatment returned the liver to its basal condition, restoring homeostasis. This analysis yielded, in the highly nonpolar organic extraction, sphingolipids and, in the more polar ACN precipitation extraction, acylcarnitine species (FIG. 4B). Each species of sphingolipid was confirmed by the presence of a fragment of 266.4 m/z for sphinganine, 264.4 for ceramides (Cer) monohexose ceramides (monohexCer), sphingosine, and sphingosine 1 phosphate (−1-P), and 184.0 for sphingomyelin (SM). While this approach was used to confirm the identification of Cer(18:1/23:0) and Cer(18:1/25:0), other possible identifications for these compounds are oxidized Cer(18:1/22:1) and Cer(18:1/24:1) species respectively. Acylcarnitine species were confirmed by an 85.0 m/z fragment. Together these results suggest a change in fat metabolism in the liver upon the administration of 11U3-2M treatment.

To confirm the changes in sphingolipids seen in the untargeted metabolomics data, we quantified the level of sphingolipid species confirmed in the Compound Discoverer analysis. The quantification of ceramide species (FIG. 4C) confirmed the significant increase of common ceramides (e.g. Cer(18:1/16:0) and Cer(18:1/20:0)) in the HFD+veh group vs LFD+veh, and their significant reduction with 11U3-2M treatment. The total ceramide levels in each treatment reflected these results (FIG. 4C). Interestingly, these effects were analogous in SM (18:1/16:0), SM (18:1/20:0), and SM(18:1/24:0) which had not previously been observed (FIG. 4C).

These results give strong evidence that 11U3-2M changes hepatic metabolism, specifically affecting FA metabolism.

### 11U3-2M Activates Pathways Related to Fatty Acids β-Oxidation and PPARα

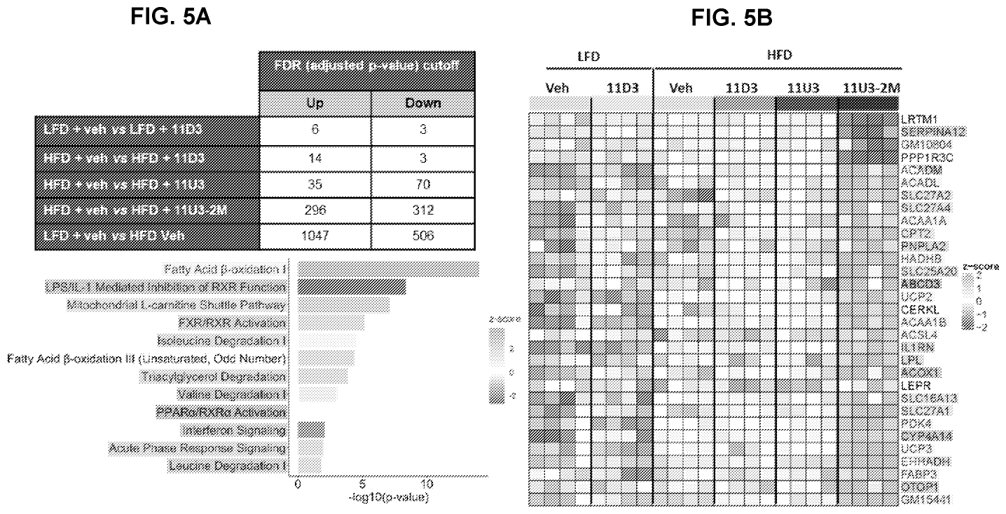

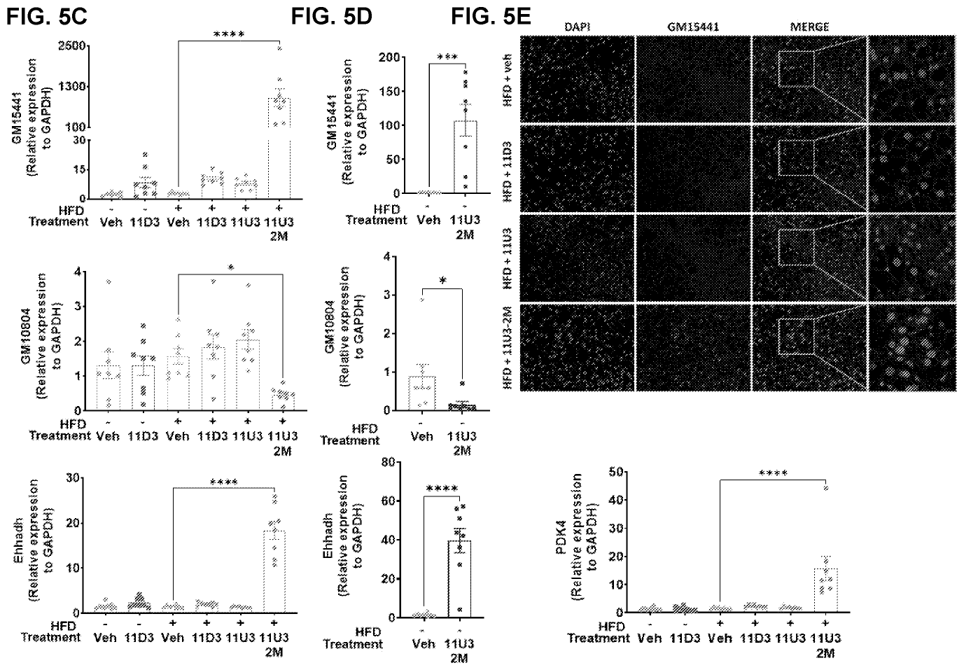

To understand the effect of FuFA, we proceed to an RNA-Sequencing (RNA-Seq) analysis on livers. The FDR cutoff showed 1047 genes upregulated and 506 genes downregulated between LFD+veh vs HFD+veh, indicating a striking effect of the diet on liver gene expression and physiology. Only a few genes were up and downregulated with the I 1D3 (14 and 3) and I 1U3 treatment (35 and 70) compared to HFD+veh. However, a greater number of genes were altered by 11U3-2M compared with HFD+veh (296 and 312, respectively) (FIG. 5A).

By using the Ingenuity Pathway Analysis (IPA) on these genes, we observed that FA β-oxidation was the most activated pathway, followed by mitochondrial L-carnitine shuttle pathway, triacylglycerol degradation, and PPARα activation. These pathways indicate a clear increase in FA oxidation mediated by 11U3-2M (FIG. 5A). In line with these data, we also observed an increase in pyruvate dehydrogenase kinase 4 (PDK4) gene (7-fold, p=3.93e-22) between 11U3-2M+veh and HFD+veh groups. PDK4 is known to be a contributor to the influential shift from glucose to FA as major energy fuel after PPARα activation, therefore increasing FA β-oxidation. RT-qPCR analysis confirmed the PDK4 increase in 11U3-2M treated livers (9.3-fold, p<0.0001) (FIG. 5C).

In addition, the most significant gene upregulated was the long non-coding RNA (LncRNA) GM15441 (137-fold increased, p=4.43e-65). This LncRNA has been recently reported to attenuate hepatic inflammasome activation in response to PPARα agonism and fasting. GM15441 was confirmed by RT-qPCR and was increased by 320-fold (p<0.0001) in 11U3-2M compared to HFD+vch. None of these genes were significantly upregulated in 11D3 and 11U3 treatments. These two highly significant genes modulated by 11U3-2M are showing an effect toward increased PPARα-dependent FA β-oxidation (FIG. 5C). To detect and locate this LncRNA in the livers, we performed an in-situ hybridization for GM15441 in HFD-treated groups. The signal for GM15441 was only detected in the nucleus of cells from 11U3-2M treated livers (FIG. 5D) confirming this massive increase of GM15441 and its effect on FA β-oxidation mediated by PPARα.

Interestingly, one of the most significant genes downregulated was the LncRNA GM10804 (5-fold, p=9.46e-4). Knockdown of GM10804 has been recently involved in the suppression of disorders of hepatic glucose and lipid metabolism in diabetes with NAFLD ³⁹. RT-qPCR confirmed that GM10804 was decreased by 3.3-fold (p<0.05) in 11U3-2M-treated HFD mice compared to HFD+veh (FIG. 5B-D).

### 11U3-2M Increases the Number of Mitochondria and Complex Activity

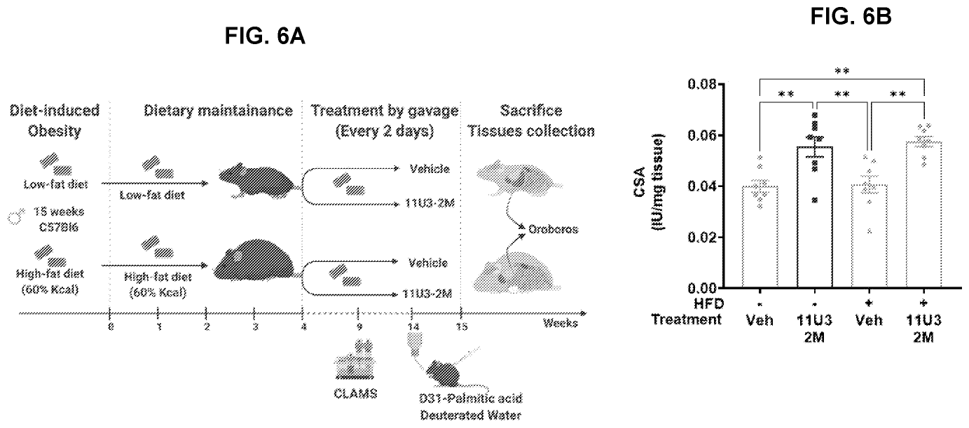

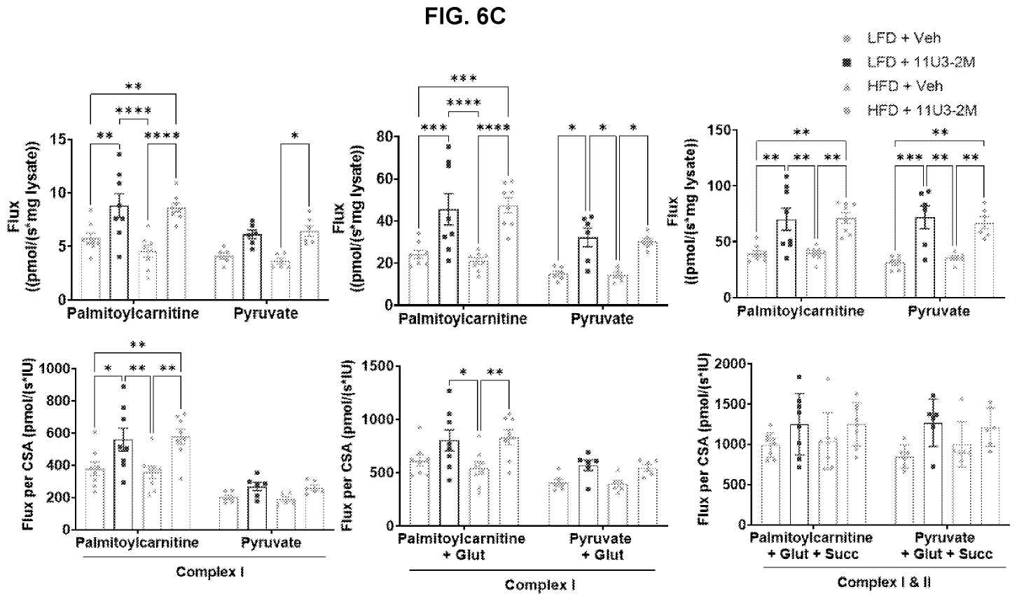

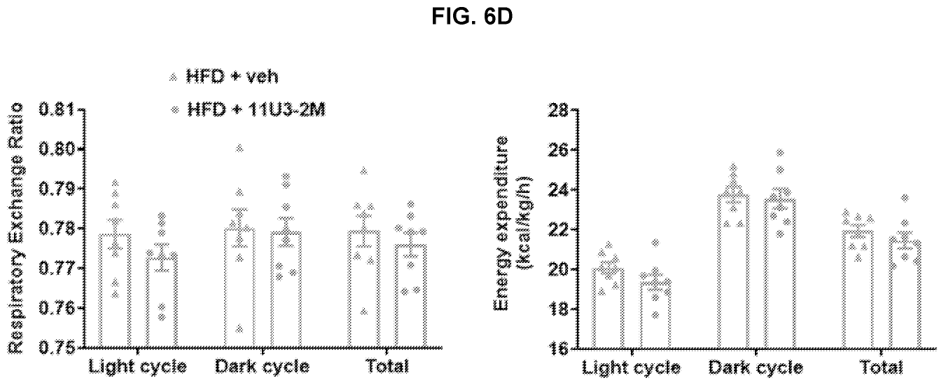

To confirm the higher FA β-oxidation rate in mice treated with 11U3-2M and investigate mitochondria function, we used a new cohort of mice under the same diet and dose of 11U3-2M as the first cohort. At the time of sacrifice, fresh liver sections were isolated and used directly for Oroboros analysis to measure oxygen consumption and mitochondria complexes activity (FIG. 6A). The maximal activity of citrate synthase (CSA) indicates the mitochondrial content of the tissue. The measurement of CSA in our groups showed a significant increase in the activity in both LFD and HFD groups treated with 11U3-2M vs corresponding veh treatments (27% and 29%, respectively, FIG. 6B). The Oroboros analysis of mitochondria flux using two different substrates (palmitoylcarnitine and pyruvate) showed a significantly higher flux in 11U3-2M treated mice (FIG. 6C). This increase can be explained by the higher number of mitochondria in the 11U3-2M treated mice. However, the flux normalized to the CSA level was still significantly increased (in LFD 32.5% and HFD 38.3% compared to the corresponding veh group), showing that 11U3-2M treatment independent of the number of mitochondria increases the flux level. This is due to a higher level of FA 3-oxidation mediated by 11U3-2M, increasing the level of NADH which is the substrate of complex I in the electron transport chain. No significant change was obtained when using pyruvate as the substrate (even though a trend was observed) indicating that 11U3-2M treated cells favored FA D-oxidation over glycolysis. These observations were similar, even though a little bit lower, after the addition of glutamate (23% for LFD+11U3-2M and 34% for HFD+11U3-2M, compared to veh treated groups with ratio to CSA). The addition of succinate, representing the maximum respiration of mitochondria didn't show any significant difference between each condition (normalized by CSA) meaning that activity of succinate dehydrogenase complex is not modified by our treatment 11U3-2M (FIG. 6C).

### Formation of 11U3-2M-CoA Adduct and its Effect on Fatty Acids β-Oxidation by Direct Inhibition of ACC and ACLY

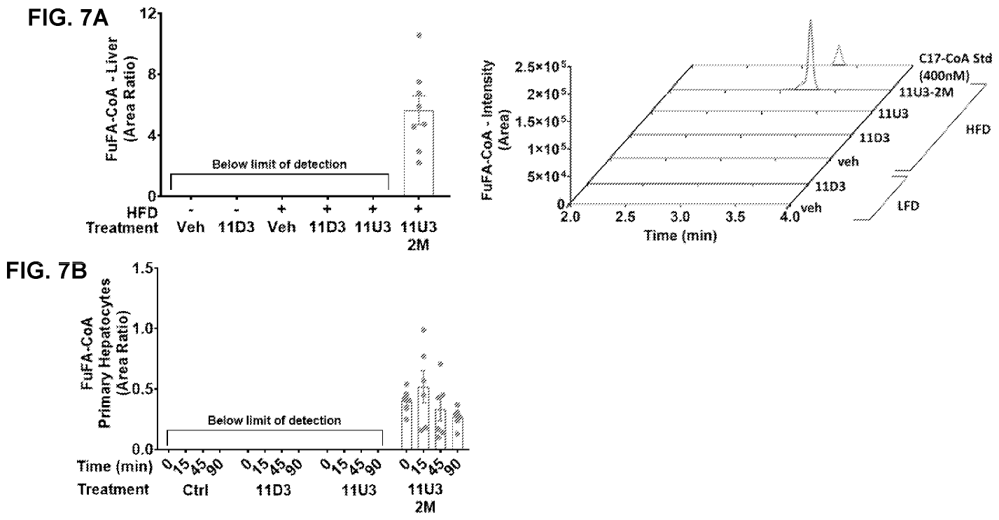

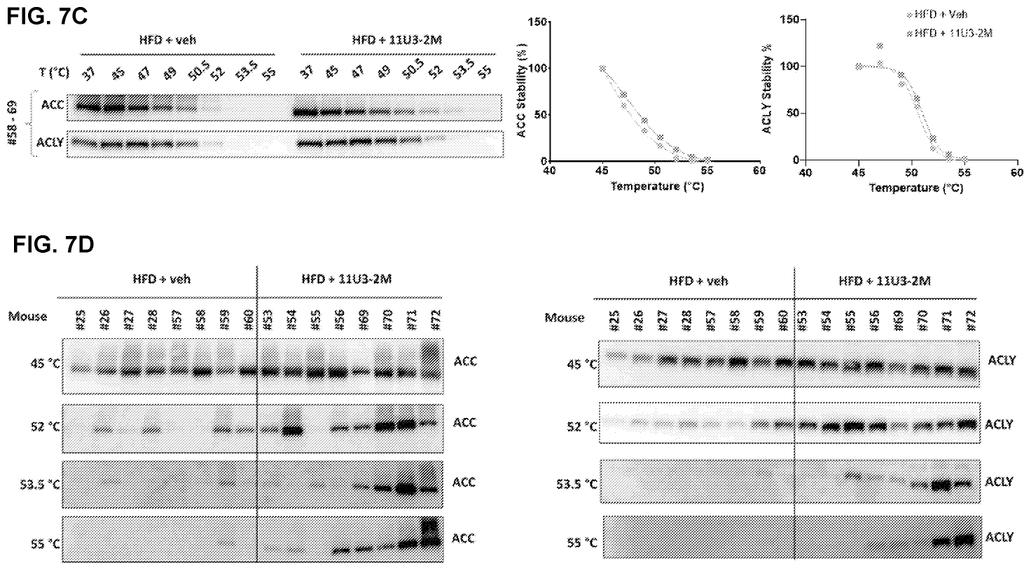

While significant effects on FA β-oxidation and PPARα pathways were observed with 11U3-2M treatment, we investigated the effects of 11U3-2M on key enzymes through the formation of its CoA adduct in vivo. The quantification of FuFA-CoA conjugates was performed on the liver samples of FuFA-treated mice using mass spectrometry. The ratios of FuFA-CoAs to the internal standard, C_{17:0}-CoA, were calculated. While veh treated groups didn't show the presence of any FuFA-CoA conjugates, we also didn't see either 11D3- or 11U3-CoA adduct (below detection limit) in the treated groups. Nevertheless, 11U3-2M-CoA was significantly elevated (FIG. 7A). We also were investigated in the formation of FuFA-CoAs in mouse primary hepatocytes. Freshly isolated hepatocytes were incubated with FuFAs for 1 h and washed with PBS. The cells were then harvested after 0, 15, 45, or 90 min. The data showed that the formation and metabolism of 11U3-2M-CoA over time, whereas no significant amount of 11D3- or 11U3-CoA was detected (FIG. 7B).

To evaluate the effect of 11U3-2M-CoA on key enzymes in the FA synthesis pathways, we proceed to a thermal shift assay to elucidate the thermal stabilization of ACC and ACLY upon 11U3-2M-CoA binding. One random mouse from HFD+veh group (#58) and HFD+11U3-2M group (#69) were used to see the general stability and to determine the best temperatures. ACC and ACLY showed higher stability in the HFD+11U3-2M treatment at 52, 53.5, and 55° C. compared to veh treatment (FIG. 7C). Full cohorts were then exposed to the critical temperatures determined in the first thermal shift assay (45° C. for total protein level and 52, 53.5, and 55° C. to analyze the stability). Mice from the HFD+11U3-2M group showed greatly increased ACC and ACLY stability at the higher temperature indicating the binding between 11U3-2M-CoA and the proteins (FIG. 7D).

These data show that 11U3-2M also inhibits key enzymes regulating FA biosynthesis and metabolism through its stable CoA conjugate.

| branched fatty acid-amino acid conjugates | Straight-chain fatty acid-amino acid conjugates |
|---|---|
| 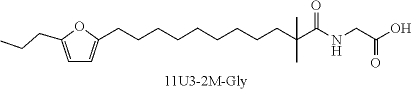 [SMILES: CCCc1ccc(CCCCCCCCCC(C)(C)C(=O)NCC(=O)O)o1] | 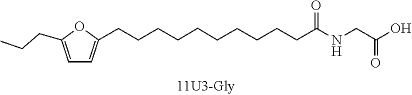 [SMILES: CCCc1ccc(CCCCCCCCCCC(=O)NCC(=O)O)o1] |
| 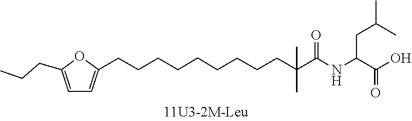 [SMILES: CCCc1ccc(CCCCCCCCCC(C)(C)C(=O)NC(CC(C)C)C(=O)O)o1] | 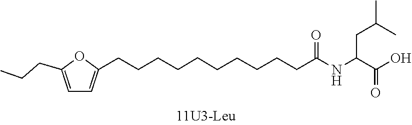 [SMILES: CCCc1ccc(CCCCCCCCCCC(=O)NC(CC(C)C)C(=O)O)o1] |
| 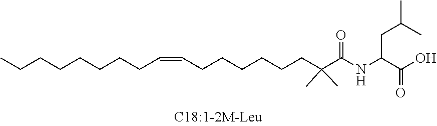 [SMILES: CCCCCCCC/C=C\CCCCCCC(C)(C)C(=O)NC(CC(C)C)C(=O)O] | 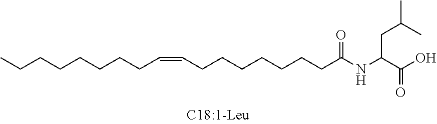 [SMILES: CCCCCCCC/C=C\CCCCCCCC(=O)NC(CC(C)C)C(=O)O] |
| 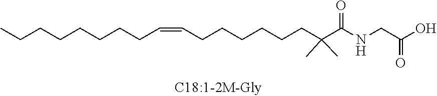 [SMILES: CCCCCCCC/C=C\CCCCCCC(C)(C)C(=O)NCC(=O)O] | 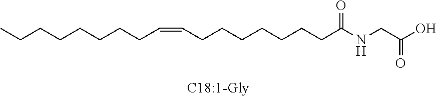 [SMILES: CCCCCCCC/C=C\CCCCCCCC(=O)NCC(=O)O] |
| 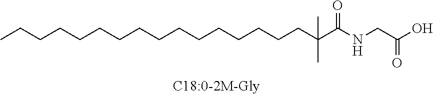 [SMILES: CCCCCCCCCCCCCCCCC(C)(C)C(=O)NCC(=O)O] | 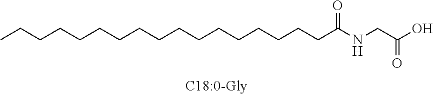 [SMILES: CCCCCCCCCCCCCCCCCC(=O)NCC(=O)O] |
| 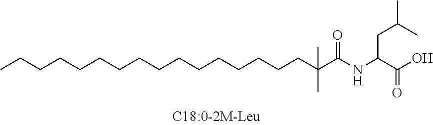 [SMILES: CCCCCCCCCCCCCCCCC(C)(C)C(=O)NC(CC(C)C)C(=O)O] | 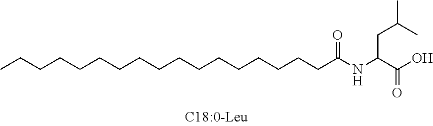 [SMILES: CCCCCCCCCCCCCCCCCC(=O)NC(CC(C)C)C(=O)O] |

Fatty acids are enzymatically converted into NAA (N-acyl amino acids, also named fatty acyl amino acids conjugates FA-AA). The Leucine and Glycine conjugate levels are reduced in metabolic diseases and are protective. As part of this invention, The FuFA or the dimethylated fatty acids with increased half-life were combined with the selected Gly and Leu amino acids to achieve additional protection.

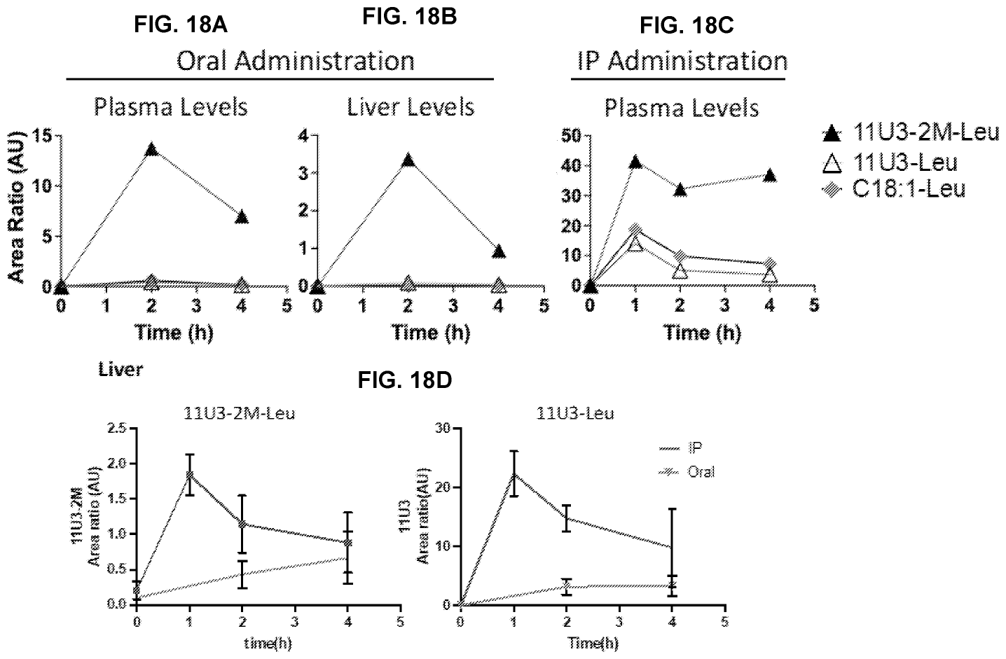

A library of branched fatty acid-amino acid (FA-AA) conjugates was synthesized, exemplified in the table above. Significantly improved oral bioavailability was observed with the dimethylated FA-AA conjugates. The absorption of the selected straight chain vs. branched FA-AA conjugates was compared in a cassette PK study as described below in more detail. A mixture of all the FA-AA conjugates (with or without branching) was concomitantly administered orally to mice (n=4-5, 30 mg/kg each compound). All the analogs with dimethyl groups installed in the fatty acid moiety exhibited >20-fold higher plasma and liver concentration upon oral administration than their straight-chain counterparts. For example, 11U3-2M-Leu exhibited greater resistance to metabolism as their plasma concentration was maintained over a longer period than 11U3-Leu, a straight-chain counterpart of 11U3-2M-Leu (FIGS. 18A-18D). The absorption improvement resulting from the branching of the fatty acids was ubiquitous across all the FA-AA conjugates independent of the choice of amino acids. In contrast, all tested FA-AA conjugated dosed intraperitoneally (n=4-5, 30 mg/kg each compound) reached significant levels in circulation, bypassing the first-pass metabolism. To note, 11U3-2M-Leu still displayed significantly higher Cₘₐₓ, AUC₀₋₄, and a slower metabolism than all the straight-chain analogs. In addition, FA-AA conjugates were found to slowly hydrolyze and release the secondary bioactive fatty acid (e.g., 11U3-2M) into the liver and plasma, achieving dual actions to protect against metabolic dysfunctions.

### Cassette PK Experiments

Two cassette PK experiments were performed to compare the pharmacokinetics of dimethyl acyl amino acids (acyl-2M-amino acids) with their natural, non-branched analogs and evaluate the increased half-life, oral bioavailability, and liver concentrations obtained by the invention (introduction of the dimethyl (2M) branching).

Groups of mice (n=4 per time point per group) were used. The compounds were delivered using a cassette pharmacokinetic design by oral gavage or by intraperitoneal injections at a dose of 30 mg/kg. Groups are detailed in table 1. To note, the methyl ester of C18:0-2M-Gly was abbreviated as C18:0-2M-Gly-Me. Plasma and liver levels were established using HPLC-mass spectrometry-based determinations and are shown in FIGS. 1-4 as relative ratios.

The following groups were used, and the time points were 0, 1, 2, and 4 h for intraperitoneal administration and 0, 2, and 4 h for oral administration.

| Group 1 | Group 2 |
|---|---|
| 11U3-Gly | 11U3-Leu |
| 11U3-2M-Gly | 11U3-2M-Leu |
| C18:1-Gly | C18:1-Leu |
| C18:0-2M-Gly | C18:0-2M-Leu |
| C18:0-2M-Gly-Me | C18:0-Leu |

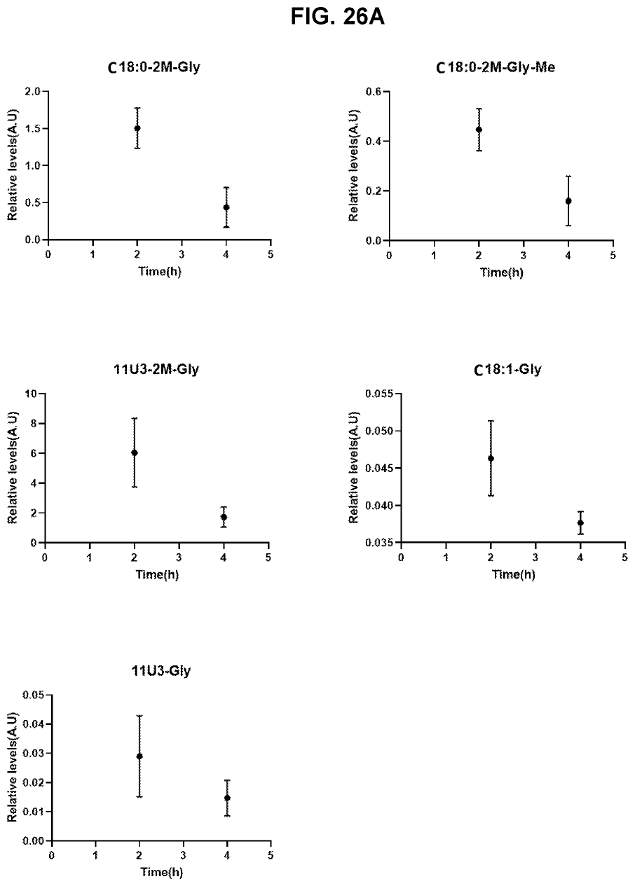

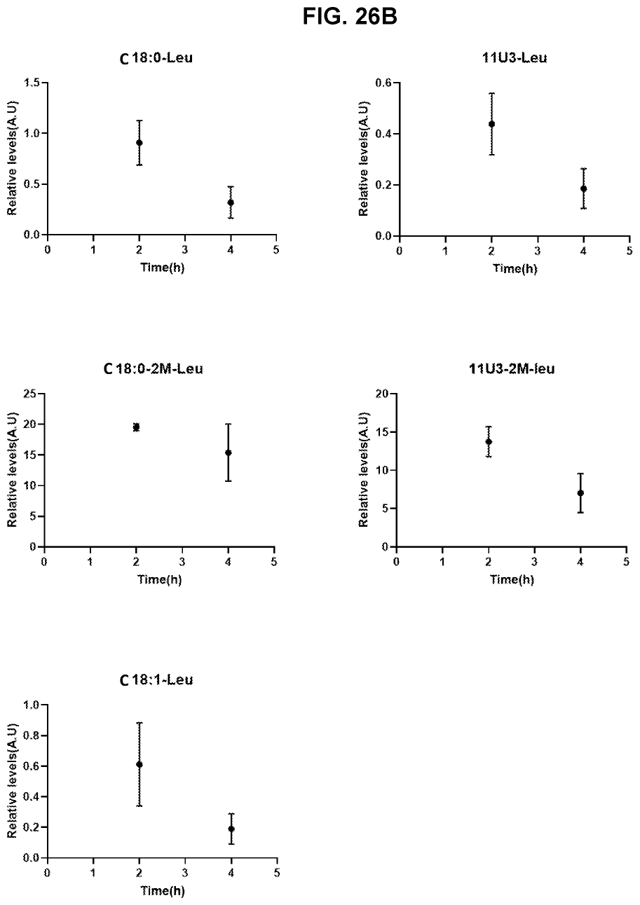

Plasma data shows that the addition of the 2M group to the acyl-amino acids makes the compounds orally bioavailable (FIGS. 26A-26B). Without the introduced modification, these compounds reach only very low levels in plasma (FIGS. 26A-26B).

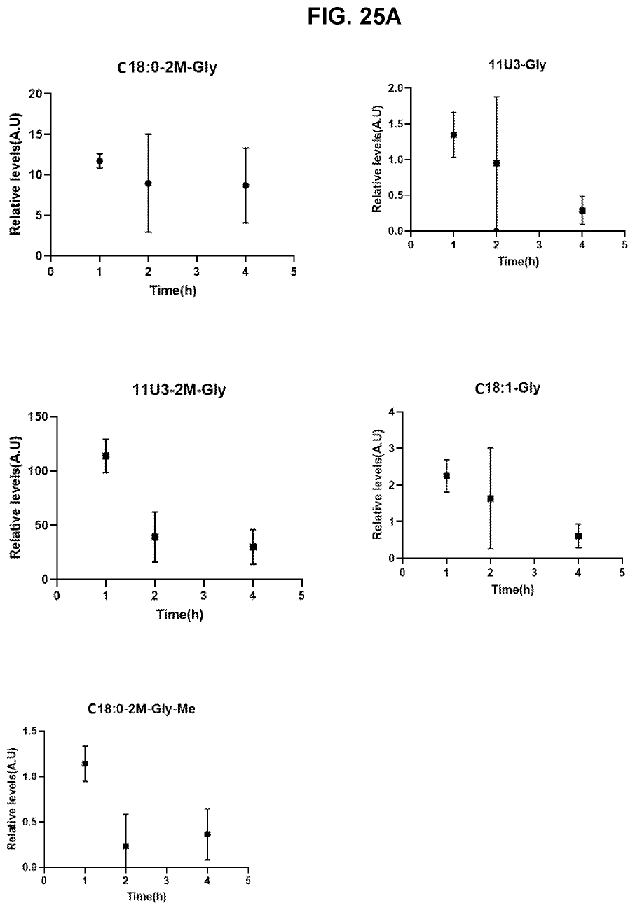

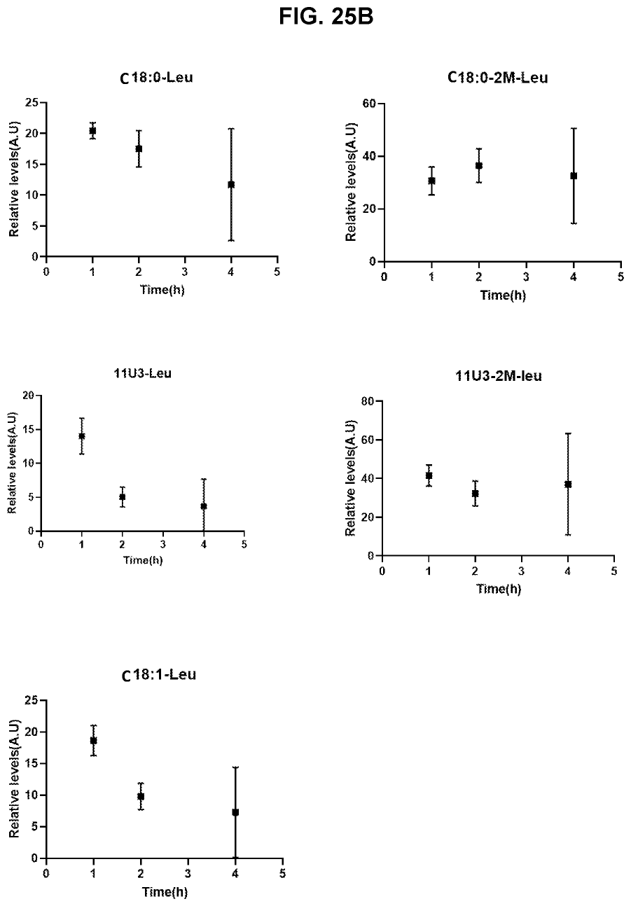

The intraperitoneal administration indicates that the compounds that contain the 2M group have a significantly increased half-life in plasma, reaching significantly higher levels (FIGS. 25A-25B).

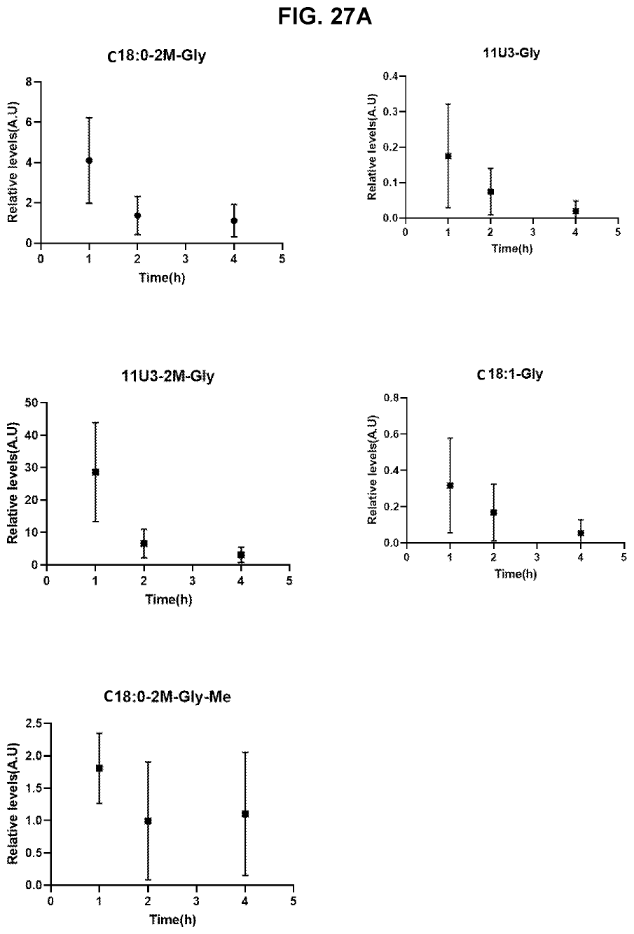

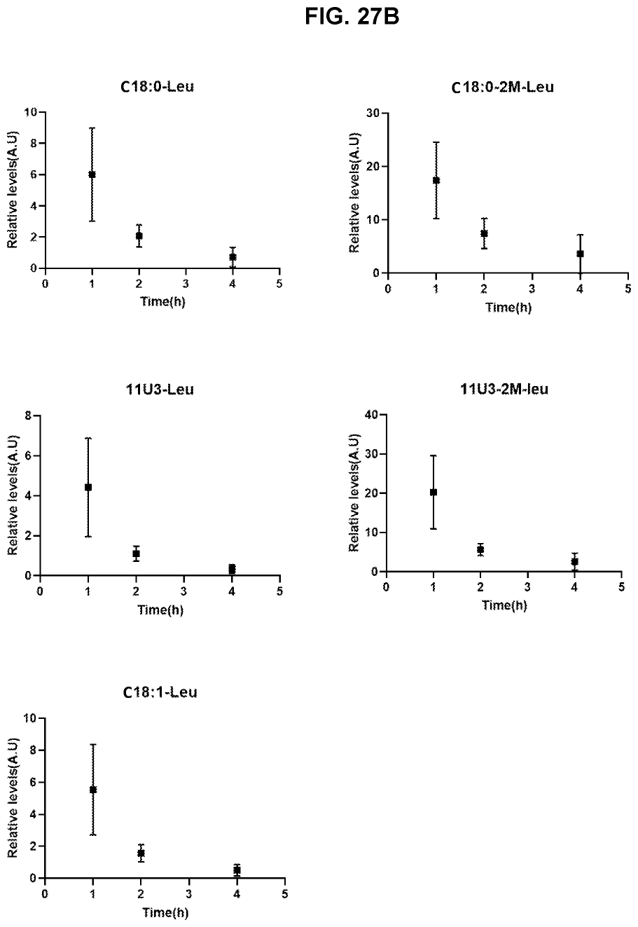

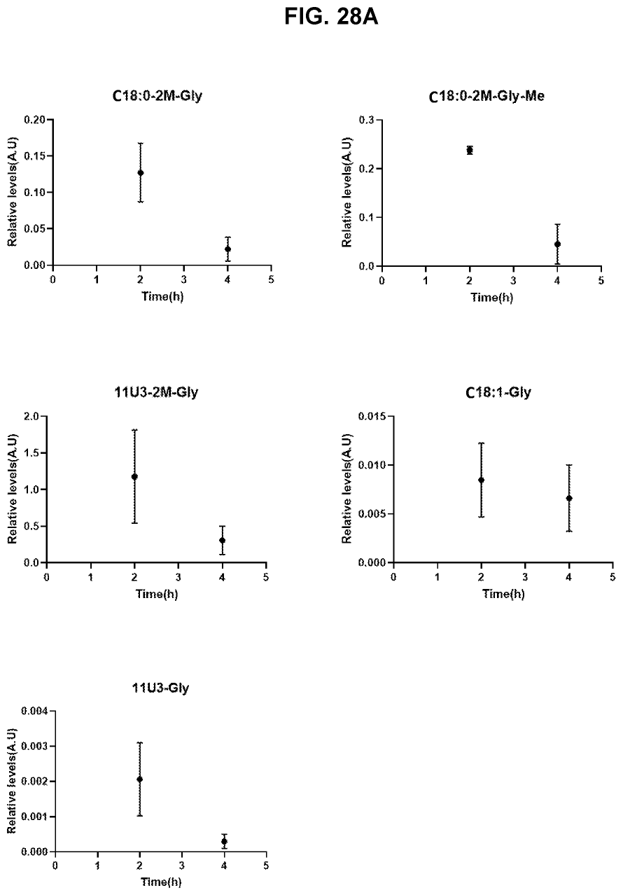

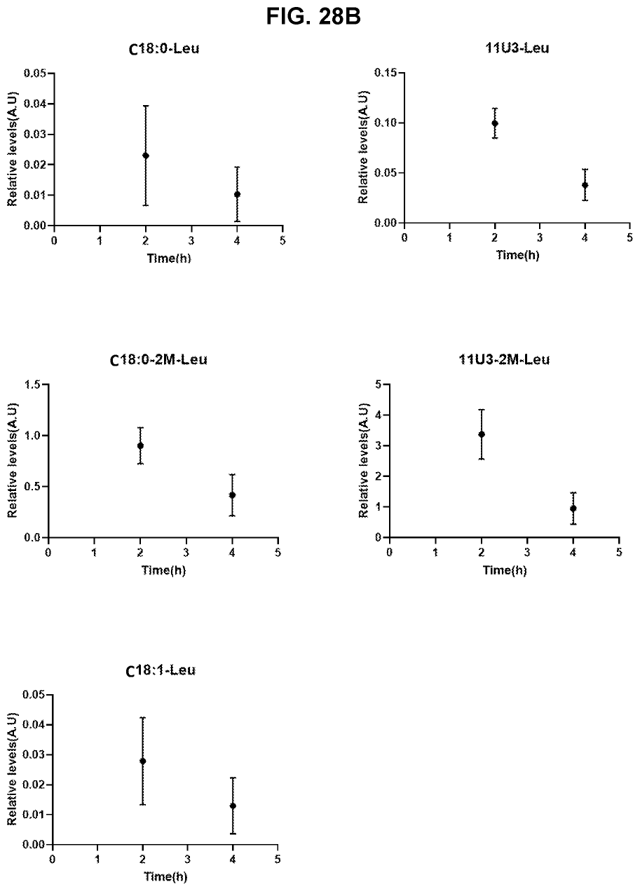

Analysis of liver tissues mirrors the effects observed in plasma, with the addition of a 2M group leading to increased tissue levels and half-life (FIGS. 27A-28B).

These effects are consistent among the groups, with the 11U3-2M-Gly and 11U3-2M-Leu reaching the highest levels in plasma and liver under both intraperitoneal and oral administration.

### Benefits in NASH Animals

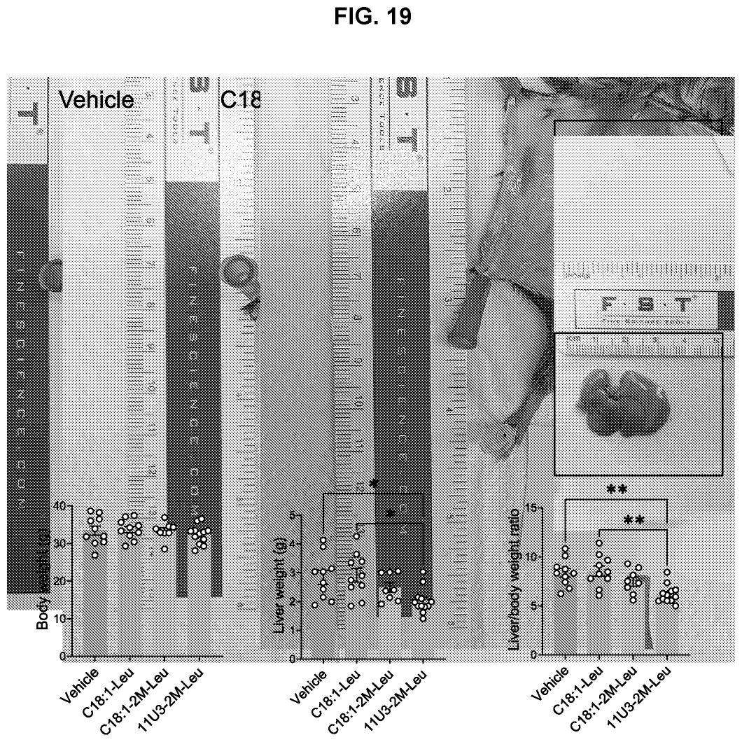

This study defined the protective properties of branched FA-AA conjugates and the enhanced potency of 11U3-2M conjugated to Leucine (Leu) when administered orally in an established NASH mouse model. C57BL6 mice were fed with the NASH diet for 16 weeks and then orally administered with vehicle (n=10), C_{18:1}-Leu (n=10), C_{18:1}-2M-Leu (n=8) and 11U3-2M-Leu (n=12) (10 mg/kg/day) for 6 weeks while on the NASH diet. Mice were sacrificed and organs were collected for further analysis. We found a significant decrease in the liver and liver/body weight ratio in the 11U3-2Me-Leu treatment group in comparison to vehicle control (FIG. 19).

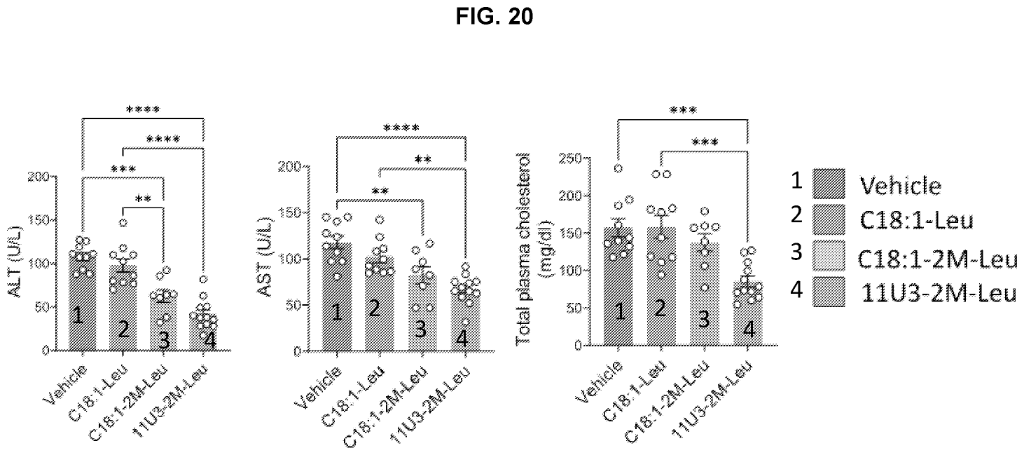

Additionally, blood biomarkers associated with liver injury, including aspartate transaminase (AST) and alanine transaminase (ALT), together with cholesterol levels were also significantly decreased in the animals treated with 11U3-2M-Leu. Plasma AST and ALT were also significantly decreased in the animals treated with C_{18:1}-2M-Leu (FIG. 20).

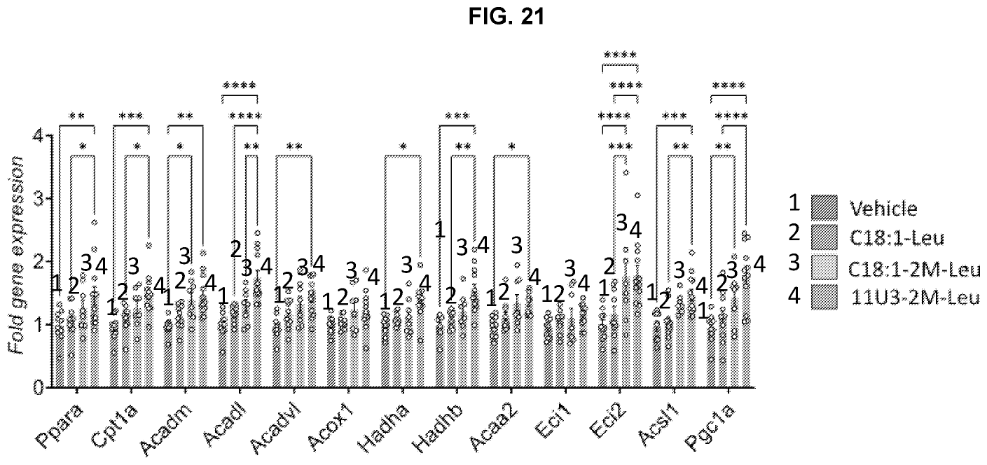

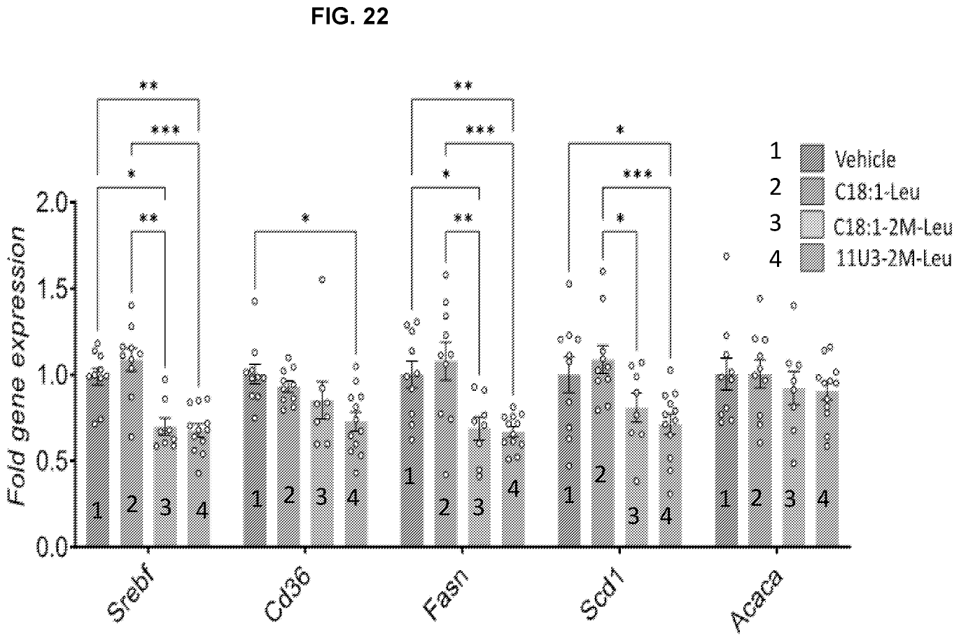

Gene expression analysis of the animal livers demonstrated that treatment with 11U3-2M-Leu, and to a lesser degree c.18:1-2M-Leu, significantly induced the expression of key genes regulating fatty acid oxidation through the PPARα signaling pathway (FIG. 21), and suppressed the expression of genes regulating lipogenesis (FIG. 22), both of which contributed to the attenuation of hepatic steatosis that is one of the hallmarks of NASH.

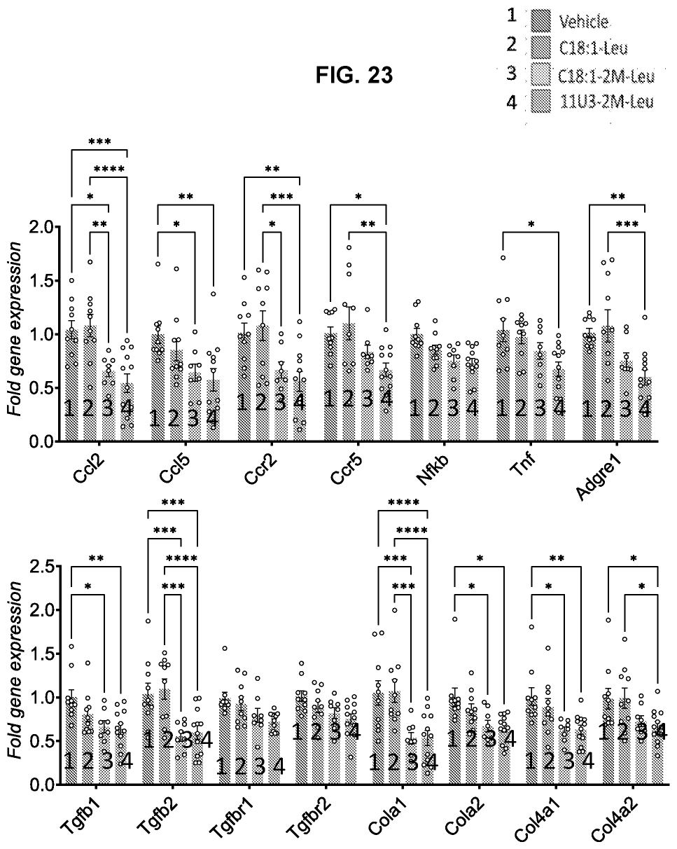

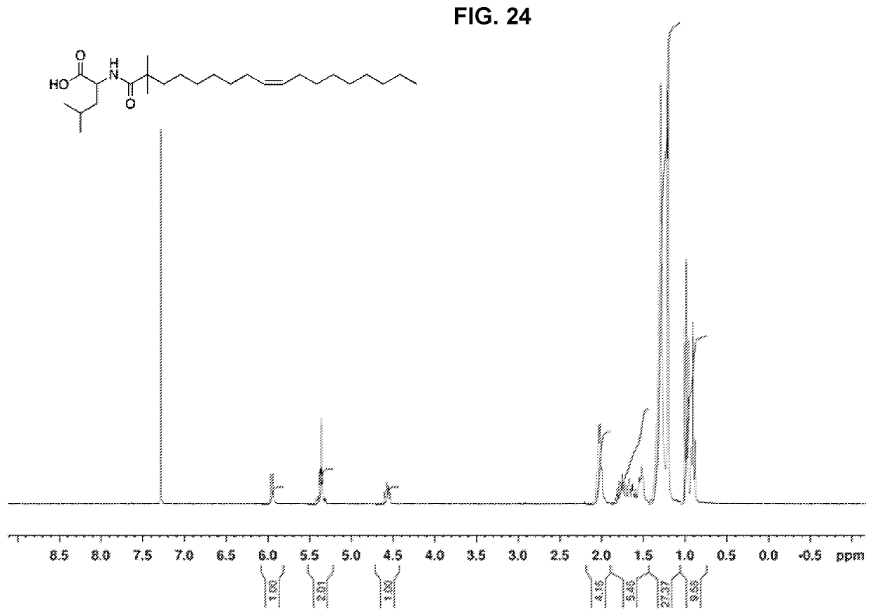

Lastly, inflammatory genes including Ccl2 and Ccl5 which are responsible for monocyte recruitment and hepatic fibrosis were found to be significantly reduced in the liver (FIG. 23). Together with the downregulation of various fibrosis and cell death genes (e.g., collagens and TGF-b), 11U3-2M-Leu and C18:1-2M-Leu treatment also demonstrated anti-fibrotic actions beyond alleviating hepatic steatosis (FIG. 24). To note, suppressing the hepatic and circulating Ccl2 benefits both NASH and atherosclerosis by preventing the recruitment of monocytes in the atherosclerotic plaque or the liver.

In view of the many possible embodiments to which the principles of the disclosed invention may be applied, it should be recognized that the illustrated embodiments are only preferred examples of the invention and should not be taken as limiting the scope of the invention.

1. A compound, or a pharmaceutically acceptable salt or ester thereof, having a structure of: 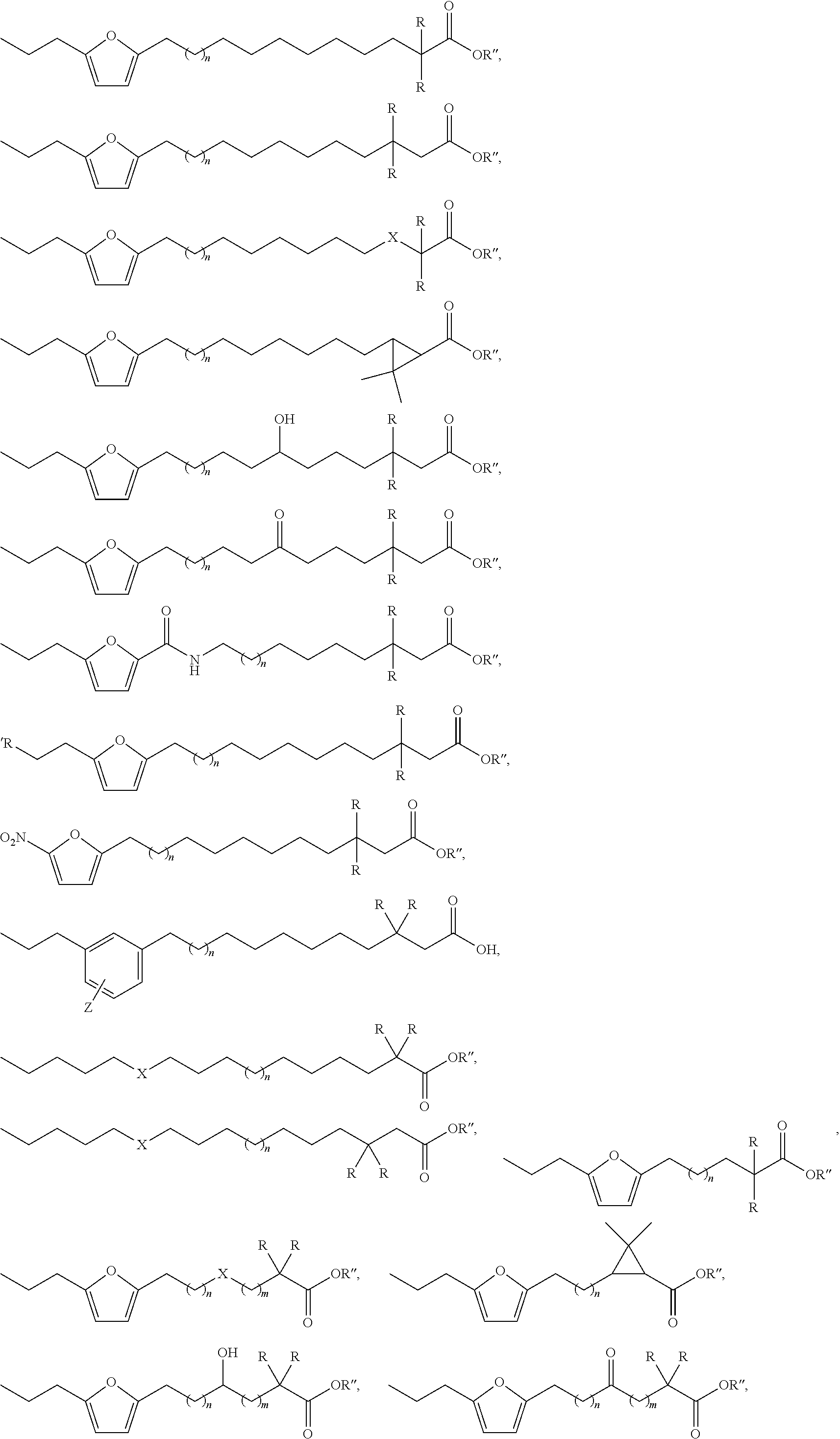 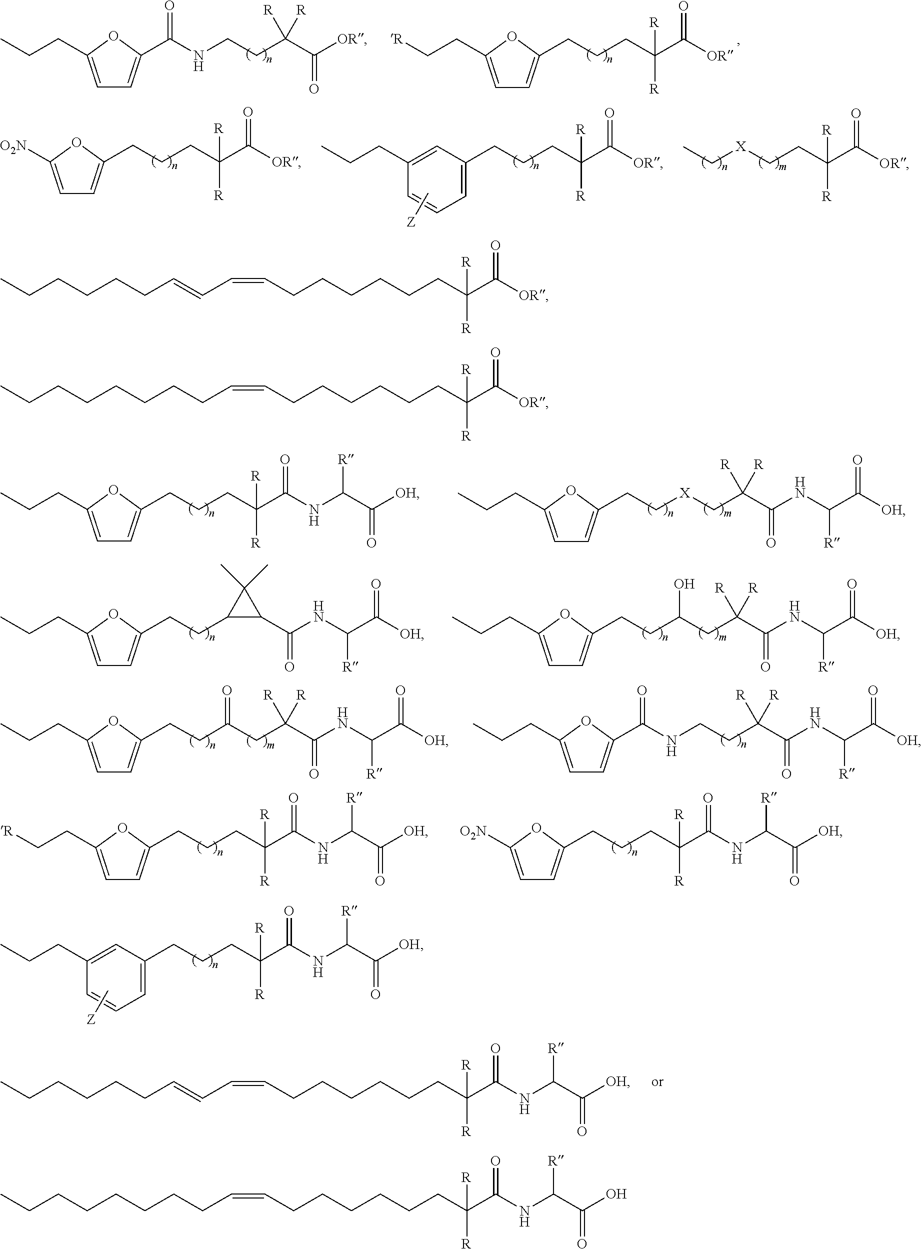
wherein each R is independently hydrogen, a C₁-C₆ alkyl, a C₁-C₆ haloalkyl, or a halogen, provided at least one R is a C₁-C₆ alkyl, a C₁-C₆ haloalkyl, or a halogen; X is O, S, or NH; ′R is a C₁-C₆ alkyl; Z is C₁-C₆ alkyl, C₁-C₆ haloalkyl, or halogen; R″ is hydrogen or alkyl; R′″ is a side chain of a natural L-amino acid; n is 1 to 10; and m is 0 to 10.

2. The compound of claim 1, having a structure of: 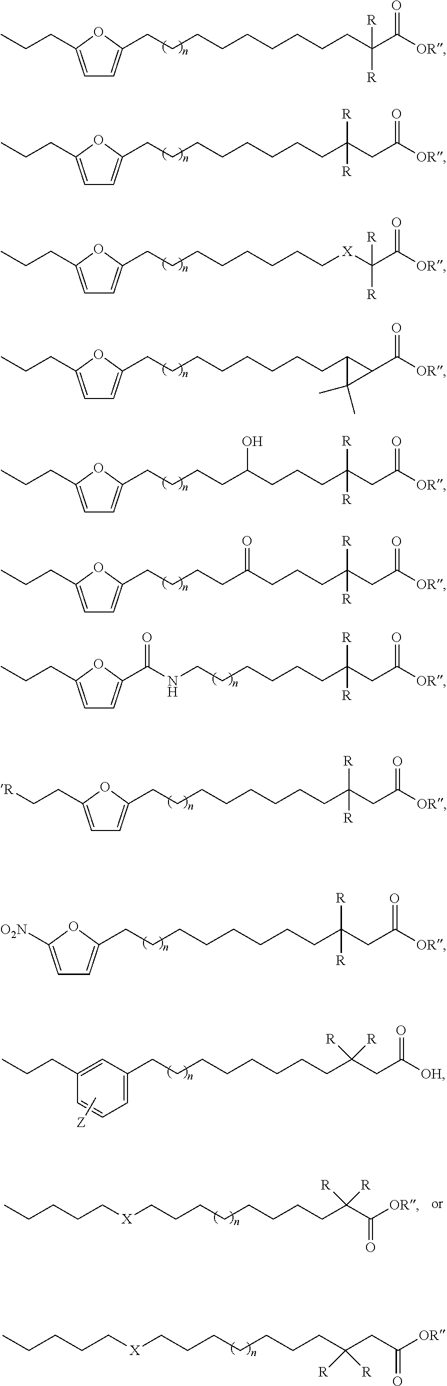
wherein each R is independently hydrogen, a C₁-C₆ alkyl, a C₁-C₆ haloalkyl, or a halogen, provided at least one R is a C₁-C₆ alkyl, a C₁-C₆ haloalkyl, or a halogen; X is O, S, or NH; ′R is a C₁-C₆ alkyl; Z is C₁-C₆ alkyl, C₁-C₆ haloalkyl, or halogen; R″ is hydrogen or alkyl; and n is 1 to 10.

3. The compound of claim 1, wherein both R groups are not hydrogen.

4. The compound of claim 1, wherein both R groups are the same.

5. The compound of claim 1, wherein each R group is independently methyl, ethyl, propyl, isopropyl, flourine or chlorine.

6. The compound of any claim 1, wherein each R group is methyl.

7. The compound of claim 1, wherein R′″ is hydrogen or isobutyl.

8. The compound of claim 1, having a structure of:

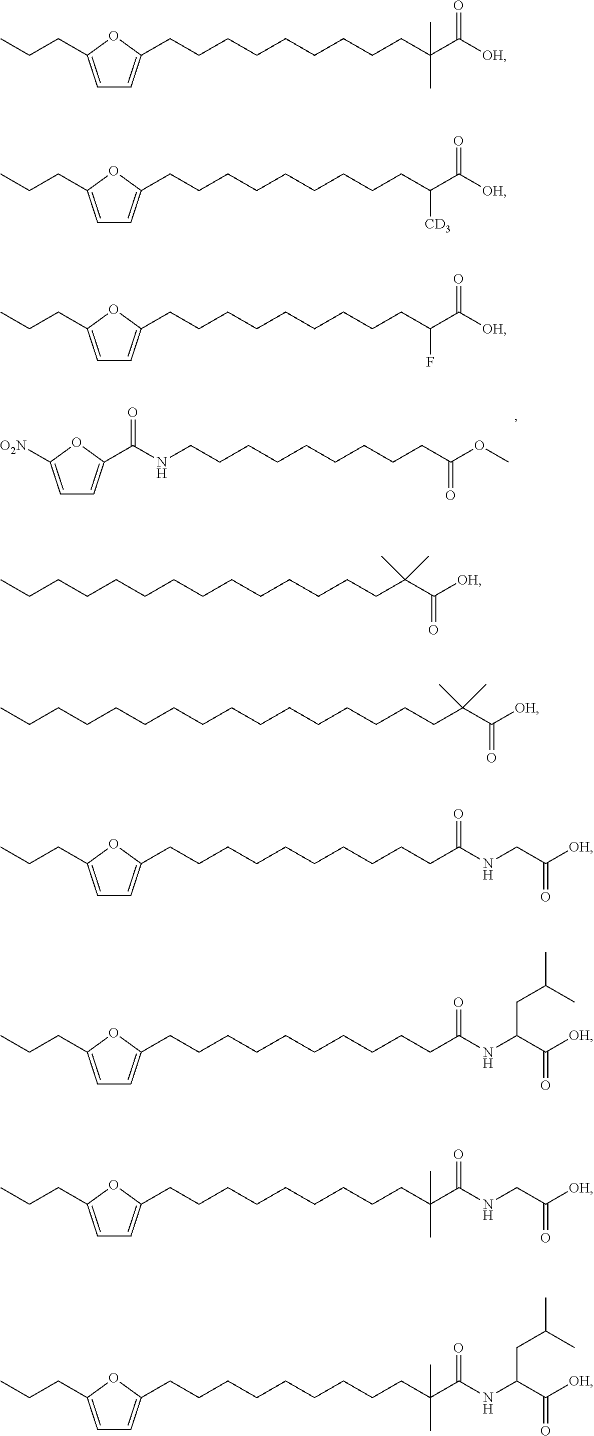

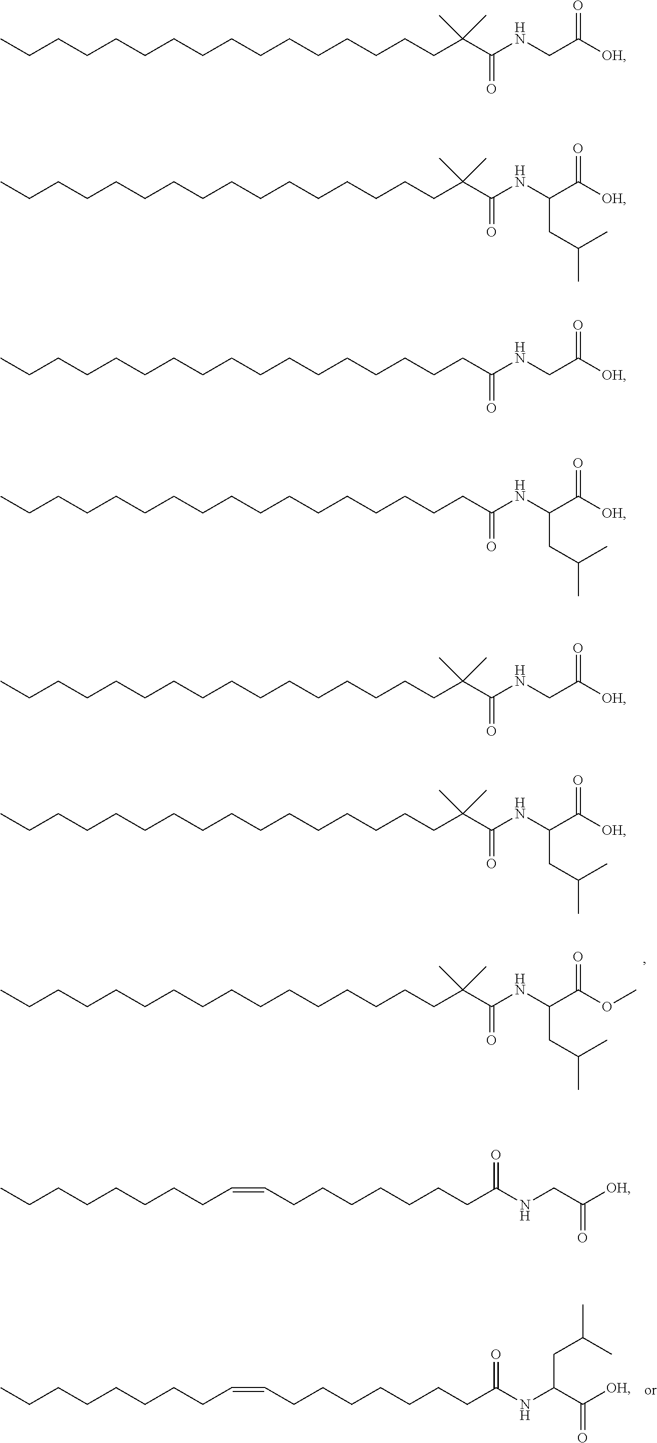

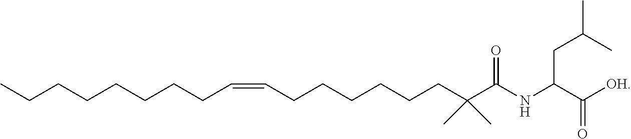 [SMILES: CCCCCCCC/C=C\CCCCCCC(C)(C)C(=O)NC(CC(C)C)C(=O)O]

9. A pharmaceutical composition comprising a compound of claim 1, and at least one pharmaceutically acceptable excipient.

10. A method comprising administering a therapeutically effective amount of a compound of claim 1 to a subject for treating metabolic syndrome in the subject.

11. A method comprising administering a therapeutically effective amount of a compound of claim 1 to a subject for treating lipid abnormalities, insulin resistance, inflammation, heart disease, cardiovascular disease (e.g., atherosclerotic cardiovascular disease (ASCVD), stroke), type 2 diabetes (T2D) mellitus, dyslipidemia, hypertriglyceridemia, immune and inflammatory conditions that require a metabolic switch from oxidative phosphorylation to glycolysis in immune and inflammatory cells, skin-associated lipid diseases, acne, hidradenitis suppurativa, osteoarthritis, Crohn's disease, psoriasis, rheumatoid arthritis, obesity-related cancer, ulcerative colitis rheumatoid arthritis, systemic lupus erythematosus, asthma, chronic obstructive pulmonary disease, chronic kidney disease, polycystic ovary syndrome (PCOS), Wilson's disease, leukemia, glioblastoma, breast cancer, pancreatic cancer, non-small cell lung cancer, lymphoma, liver cancer, lung cancer, colorectal cancer, melanoma, kidney cancer, osteosarcoma, glioblastoma, multiple sclerosis, Parkinson's disease, or amyotrophic lateral sclerosis in the subject.

12. A method comprising administering a therapeutically effective amount of a compound of claim 1 to a subject for treating a non-alcoholic fatty liver disease (NAFLD) in the subject.

13. A method comprising administering a therapeutically effective amount of a compound of claim 1 to a subject for treating nonalcoholic steatohepatitis (NASH) in the subject.

14. A method comprising administering a therapeutically effective amount of a compound of claim 1 to a subject for treating TG-induced pancreatitis and monogenetic dyslipidemia in the subject.

15. A method comprising co-administering a compound of claim 1, and at least one of fish oil and omega-3, to a subject.
